(19) United States
(12) Patent Application Publication
(10) Pub. No.: US 2026/0232607 A1
(43) Pub. Date: Aug. 13, 2026

(54) COMPOSITIONS AND METHODS INVOLVING ISOLATED COMPOUNDS

(71) Applicant: CuraSen Therapeutics, Inc., San Carlos, CA (US)

(72) Inventors: Anthony P. Ford, San Carlos, CA (US); Steven A. Smith, San Carlos, CA (US); Wei Chen, San Carlos, CA (US); Rachel A. Matt, San Carlos, CA (US); Frederick G. Westhorpe, San Carlos, CA (US); Joel R. Gever, San Carlos, CA (US)

(21) Appl. No.: 18/875,661

(22) PCT Filed: Jun. 20, 2023
(86) PCT No.: PCT/US2023/068750

§ 371 Date: Dec. 16, 2024

Related U.S. Application Data
(60) Provisional application No. 63/354,152, filed on Jun. 21, 2022.

(51) Int. Cl. A61K 31/138; A61K 31/137; A61K 45/06

(52) U.S. Cl. CPC A61K 31/138; A61K 31/137; A61K 45/06

(57) ABSTRACT

In various aspects and embodiments provided are compositions and methods for treating subjects in need thereof. In some embodiments, the compositions and methods involve purified or isolated isomers of nadolol. In one aspect, the disclosure provides a method including identifying a subject desiring improvement of cognitive function and/or treatment of a neurodegenerative disease or disorder and administering to said subject a pharmaceutical composition of the present disclosure.

### CROSS-REFERENCE TO RELATED APPLICATION

This application claims the benefit of priority under 35 U.S.C. § 119(e) of U.S. Provisional Patent Application No. 63/354,152 filed Jun. 21, 2022. The disclosure of the prior application is considered part of and is hereby incorporated by reference in its entirety.

### FIELD OF THE INVENTION

The present disclosure relates generally to compositions and methods that can be used as therapeutics.

### BACKGROUND

Nadolol is β-adrenergic receptor (β-AR) antagonist, or β-blocker. Nadolol has been approved in many countries to treat angina pectoris, heart failure, arrhythmias and/or hypertension.

PCT Application WO2019/241736 (Ford) discloses “compositions and methods for improving cognition and/or treating a neurodegenerative disease in a patient” and that the methods may “. . . include identifying a patient in need of, or desiring improvement of, cognitive function and/or treatment of a neurodegenerative disease and administering to the patient a β agonist and optionally a peripherally acting β-blocker (PABRA).” Ford further discloses that “[e]xamples of selective peripherally acting b-blockers (PABRA) that may in certain embodiments be used in the methods disclosed herein include nadolol, atenolol, sotalol and labetalol.”

PCT Application WO/2018/195473 (Scherzer) discloses “[a] method of treating a subject who has a synucleinopathy, the method comprising: administering to a subject in need of such treatment therapeutically effective amounts of a β2-adrenoreceptor agonist and at least one therapeutic agent selected from the group consisting of: a synucleinopathy therapeutic agent, a β2-adrenoreceptor antagonist and a health supplement, . . . to thereby treat Parkinson's disease in the subject . . . wherein the β2-adrenoreceptor antagonist is selected from the group consisting of carteolol, carvedilol, labetalol, nadolol, penbutolol, pindolol, sotalol, timolol, oxprenolol and butaxamine.”

### SUMMARY

The present disclosure is based, at least in part, on the finding that certain isomers of nadolol have improved properties over other isomers and/or a racemic mixture of nadolol isomers. PCT/US2020/60565 discloses the use of subtherapeutic doses of PABRAs and their use in combination with beta-AR agonists in treating cognitive and neurological conditions. The dosing and other aspects of that application are, in some embodiments, applicable to the compositions and methods disclosed herein and are hereby incorporated by reference in their entirety.

Current commercial nadolol formulations are a mixture of four nadolol isomers as follows:

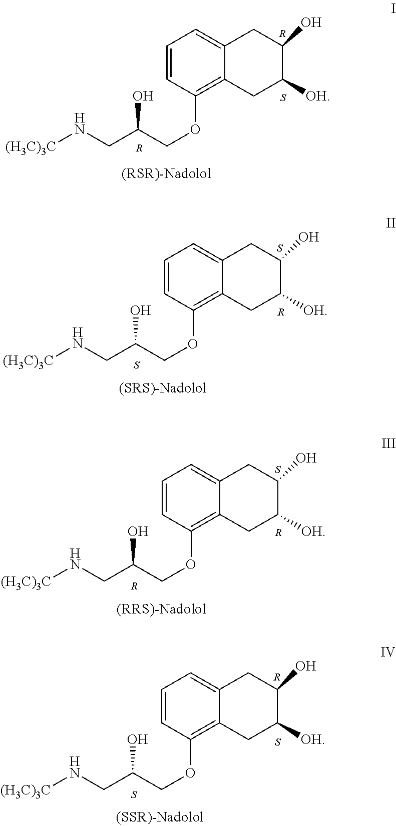

As used herein, the term “isomer” or “isomers” means chemical compounds that have identical number of atoms of each element and have the same atoms or isotopes connected by bonds of the same type but differ in their relative positions in space, apart from rotations. For this disclosure, isomer[s], diastereomer[s] and enantiomer[s] have the same meaning and may be used interchangeably.

The disclosure is based, at least in part, on the finding that different nadolol isomers may have different activities in occupying β-adrenoreceptors in the CNS which may be at least partially a result of different abilities of nadolol isomers to cross the blood brain barrier and/or the rate at which different nadolol isomers are cleared from the CNS.

In some aspects and embodiments, the present disclosure is further based at least in part on the finding that certain nadolol isomers have lower ability to cross the blood brain barrier or otherwise occupy receptors in the CNS as compared to other nadolol isomers. In many embodiments, it is preferable for the actions of nadolol to be predominantly in the periphery (i.e., outside of the CNS) with a lower relative activity in the periphery. For example, in some embodiments, nadolol (or a nadolol isomer as contemplated herein) is administered in conjunction with a β-AR agonist with the intention of preventing undesirable side effects of the β-AR agonist in the periphery while permitting the desirable actions of the β-AR agonist (such as improving cognition and/or treating a neurodegenerative condition) in the CNS. In such embodiments, nadolol formulations (such as isolated nadolol isomer formulations) that are relatively less active in the CNS (such as by decreased CNS penetrability and/or increased active transport out of the CNS) are advantageous. Accordingly, in certain embodiments, a purified nadolol isomer is preferred due to a decrease in its relative activity in the CNS as compared to the periphery (i.e. outside of the CNS). In some embodiments a preferred nadolol isomer is preferred due to improved properties with regard to receptor occupancy on beta-receptors in the CNS. In some embodiments, a purified nadolol isomer is preferred due to improved properties with regard to improved properties to CNS penetrability (e.g., improved properties being lower CNS penetrability). In some embodiments, a purified nadolol isomer is preferred due to improved properties with regard to improved properties with regard to CNS transport out of the CNS (e.g., improved properties being increased rate of transport out of the CNS). In some embodiments, a purified nadolol isomer is preferred due to improved properties with regard to improved properties to CNS penetrability (e.g., improved properties being lower CNS penetrability and improved properties with regard to CNS transport out of the CNS (e.g., improved properties being increased rate of transport out of the CNS). In some embodiments, a purified nadolol isomer is preferred due to improved properties with regard to receptor occupancy on beta-receptors in the CNS; wherein the improved CNS beta receptor occupancy properties are a result of improved CNS penetrability properties and/or improved CNS transport properties.

Accordingly, in various aspects of the disclosure pharmaceutical formulations are provided that include a purified nadolol isomer and methods of using such. In related aspects and embodiments formulations are provided that include an isomer of nadolol that is substantially free of one or more (i.e., substantially free of at least one, or at least two, or at least three) other nadolol isomers. As used herein, a “purified nadolol isomer”, or “isolated nadolol isomer” means a one or more nadolol isomer[s] that has been purified from, or isolated from, one or more other nadolol isomers; or one or more nadolol isomer[s] that is otherwise partially or substantially free of one or more other nadolol isomers; or one or more nadolol isomer[s] that is synthesized or manufactured partially or substantially free of one or more other nadolol isomers. In some embodiments, a “purified nadolol isomer”, or “isolated nadolol isomer” means a nadolol isomer that has been purified from, or isolated from, all other nadolol isomers; or a nadolol isomer that is otherwise partially or substantially free of all other nadolol isomers. In various embodiments, a purified nadolol isomer or isolated nadolol isomer may be a formulation having one or more desired nadolol isomers, wherein the total nadolol in the formulation includes less than 35%; or less than 30%; or less than 25%; or less than 20%; or less than 15%; or less than 12%; or less than 10%; or less than 9%; or less than 8%; or less than 7%; or less than 6%; or less than 5%; or less than 4%; or less than 3%; or less than 2%; or less than 1%; or less than 0.5% of the one or more of the less desired nadolol isomers. In some embodiments, a purified nadolol isomer or isolated nadolol isomer may be a formulation having one nadolol isomer, wherein the total nadolol in the formulation includes less than 35%; or less than 30%; or less than 25%; or less than 20%; or less than 15%; or less than 12%; or less than 10%; or less than 9%; or less than 8%; or less than 7%; or less than 6%; or less than 5%; or less than 4%; or less than 3%; or less than 2%; or less than 1%; or less than 0.5% of one or more of other isomers. The term “purified nadolol isomer” does not necessarily infer that a particular nadolol isomer (or isomers) were necessarily part of a mixture of additional isomers that were subjected to some sort of purification or isolation process, instead a purified nadolol isomer could in some embodiment refer to one or more nadolol isomers that were synthesized such that the desired isomer[s] was never in solution with unspecified isomers. In some embodiments, a nadolol isomer that is “substantially free” of another isomer, means one or more nadolol isomers in a formulation, wherein the formulation includes less than 35%; or less than 30%; or less than 25%; or less than 20%; or less than 15%; or less than 12%; or less than 10%; or less than 9%; or less than 8%; or less than 7%; or less than 6%; or less than 5%; or less than 4%; or less than 3%; or less than 2%; or less than 1%; or less than 0.5% of the one or more of the less desired nadolol isomers. In certain embodiments, a nadolol isomer that is “substantially free” of another isomer, means a nadolol isomer in a formulation, wherein the formulation includes less than 35%; or less than 30%; or less than 25%; or less than 20%; or less than 15%; or less than 12%; or less than 10%; or less than 9%; or less than 8%; or less than 7%; or less than 6%; or less than 5%; or less than 4%; or less than 3%; or less than 2%; or less than 1%; or less than 0.5% of all other nadolol isomers.

As used herein, the term “NI-A” or “(RSR)-NADOLOL” means the (RSR)-nadolol isomer shown above. As used herein, the term “NI-B” or “(RRS)-NADOLOL” means the (RRS)-nadolol isomer shown above. As used herein, the term “NI-C” or “(SRS)-NADOLOL” means the (SRS)-nadolol isomer shown above. In some embodiments, the term “NI-D” or “(SSR)-NADOLOL” means the (SSR)-nadolol isomer shown above.

In one aspect, a NI-D pharmaceutical formulation is provided wherein the formulation includes a purified NI-D nadolol isomer. In a related aspect, provided is a NI-D pharmaceutical formulation having the NI-D nadolol isomer that is substantially free of other nadolol isomers. In some embodiments, the NI-D formulation is substantially free of one or more nadolol isomers selected from the group selected from NI-A, NI-B, and NI-C. In some embodiments, the NI-D formulation is substantially free of the NI-A isomer. In some embodiments, the NI-D formulation is substantially free of the NI-B isomer. In some embodiments, the NI-D formulation is substantially free of the NI-C isomer. In some embodiments, the NI-D formulation is substantially free of each of the NI-A, NI-B, and NI-C nadolol isomers. In some embodiments, the NI-D formulation is substantially free of the NI-A isomer and the NI-B isomer.

In one aspect, a NI-C pharmaceutical formulation is provided wherein the formulation includes a purified NI-C nadolol isomer. In a related aspect, provided is a NI-C pharmaceutical formulation having the NI-C nadolol isomer that is substantially free of other nadolol isomers. In some embodiments, the NI-C formulation is substantially free of one or more nadolol isomers selected from the group selected from NI-A, NI-B, and NI-D. In some embodiments, the NI-C formulation is substantially free of the NI-A isomer. In some embodiments, the NI-C formulation is substantially free of the NI-B isomer. In some embodiments, the NI-C formulation is substantially free of the NI-D isomer. In some embodiments, the NI-C formulation is substantially free of each of the NI-A, NI-B, and NI-D nadolol isomers. In some embodiments, the NI-C formulation is substantially free of the NI-A isomer and the NI-B isomer.

In one aspect, a NI-C/D pharmaceutical formulation is provided wherein the formulation includes a mixture of purified NI-C and NI-D nadolol isomers. In a related aspect, provided is a NI-C/D pharmaceutical formulation having the NI-C and NI-D nadolol isomers that is substantially free of other nadolol isomers. In some embodiments, the NI-C/D formulation is substantially free of one or more nadolol isomers selected from the group selected from NI-A and NI-B. In some embodiments, the NI-C/D formulation is substantially free of the NI-A isomer. In some embodiments, the NI-C/D formulation is substantially free of the NI-B isomer. In some embodiments, the NI-C/D formulation is substantially free of the NI-A isomer and the NI-B isomer.

In one aspect, a NI-B pharmaceutical formulation is provided wherein the formulation includes a purified NI-B nadolol isomer. In a related aspect, provided is a NI-B pharmaceutical formulation having the NI-B nadolol isomer that is substantially free of other nadolol isomers. In some embodiments, the NI-B formulation is substantially free of one or more nadolol isomers selected from the group selected from NI-A, NI-C, and NI-D. In some embodiments, the NI-B formulation is substantially free of the NI-A isomer. In some embodiments, the NI-B formulation is substantially free of the NI-C isomer. In some embodiments, the NI-B formulation is substantially free of the NI-D isomer. In some embodiments, the NI-B formulation is substantially free of each of the NI-A, NI-C, and NI-D nadolol isomers.

In one aspect, a NI-A pharmaceutical formulation is provided wherein the formulation includes a purified NI-A nadolol isomer. In a related aspect, provided is a NI-A pharmaceutical formulation having the NI-A nadolol isomer that is substantially free of other nadolol isomers. In some embodiments, the NI-A formulation is substantially free of one or more nadolol isomers selected from the group selected from NI-B, NI-C, and NI-D. In some embodiments, the NI-A formulation is substantially free of the NI-B isomer. In some embodiments, the NI-A formulation is substantially free of the NI-C isomer. In some embodiments, the NI-A formulation is substantially free of the NI-D isomer. In some embodiments, the NI-A formulation is substantially free of each of the NI-B, NI-C, and NI-D nadolol isomers.

The term “NI-X” as used herein means any one of the NI-A, NI-B, NI-C, NI-D, or NI-C/D formulations as provided herein.

In one aspect, provided is a method that includes administering a nadolol formulation (such as a purified nadolol formulation) as provided herein to a subject. In one embodiment, provided is a method that includes administering a NI-D formulation as provided herein to a subject. In one embodiment, provided is a method that includes administering a NI-C formulation as provided herein to a subject. In one embodiment, provided is a method that includes administering a NI-B formulation as provided herein to a subject. In one embodiment, provided is a method that includes administering a NI-A formulation as provided herein to a subject.

In some embodiments a nadolol purified nadolol composition as provided herein is administered to a subject in combination with a second active ingredient. In one embodiment, active compounds of a combination composition are combined together in a single unit dose formulation and optionally include a pharmaceutically acceptable carrier. In another embodiment of a combination composition, the active ingredients can be administered separately, sequentially or concomitantly.

In yet another embodiment, the present invention provides a pharmaceutical composition suitable for reducing food-drug interaction and/or drug-drug interaction resulting from the administration of nadolol comprising the (SSR)-nadolol and optionally a pharmaceutically acceptable carrier.

In some embodiments, the compositions and methods disclosed herein involve the use of a purified nadolol isomer (NI-X) to treat a disease, disorder or condition. In some embodiments, the compositions and methods disclosed herein involve the use of a purified nadolol isomer (NI-X) to treat a disease, disorder or condition selected from the group consisting of angina pectoris, hypertension, a neurological disease or disorder, Anxiety, Atrial Fibrillation, Ventricular Arrhythmias Due to Congenital Long QT Syndrome, Ventricular Premature Beat, Catecholaminergic Polymorphic Ventricular Tachycardia, Supraventricular Tachycardia, Congestive Heart Failure, Heart failure with reduced ejection fraction (HFrEF), Heart failure with preserved ejection fraction (HFpEF), Acute decompensated HF, Right-sided or Left-sided Heart Failure and Gastroesophageal Variceal Hemorrhage Prophylaxis in Patients with Liver Cirrhosis, Thyrotoxicosis. In some embodiments the compositions and methods disclosed herein involve the administration of a purified nadolol isomer (NI-X) to a patient desiring an improvement in cognition.

In some embodiments, the compositions and methods provided herein involve the administration of a purified nadolol isomer to a subject desiring an improvement in cognition. In some embodiments, the compositions and methods provided herein involve the use of a purified nadolol isomer for treatment of a patient or subject identified as having a neurodegenerative disease or disorder. In some embodiments of the aspects and embodiments provided herein, the patient is identified as having a neurodegenerative disease that is one or more selected from the group consisting of MCI (mild cognitive impairment), aMCI (amnestic MCI), ALS (amyotrophic lateral sclerosis), Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), ADHD (attention deficit hyperactivity disorder), Alzheimer's disease (AD), early AD, and Down Syndrome (DS). In some embodiments the of the patient is identified as having a neurodegenerative disease that is one or more selected from the group consisting of MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), and ADHD (attention deficit hyperactivity disorder). In some embodiments the patient does not have Alzheimer's disease (AD). In some embodiments the subject does not have Down Syndrome. In some embodiments the subject does not have Parkinson's disease. In some embodiments the subject does not have dementia with Lewy bodies. In some embodiments the patient or subject has not been diagnosed with any neurodegenerative disease or disorder, for example a subject that has not been diagnosed with a disease or disorder listed above.

As used herein, the term “patient” can be used interchangeably with “subject” and refers to an individual that receives a composition or treatment as disclosed herein or is subjected to a method of the disclosure. In certain embodiments a patient or subject may have been diagnosed with a condition, disease or disorder and a composition or method of the disclosure is administered/applied with the intention of treating condition, disease or disorder. In some embodiments a patient or subject is any individual that receives a composition or method of the disclosure and has not necessarily been diagnosed with any particular condition, disease or disorder. In some embodiments, a patient or subject is any individual desiring an improvement in cognition or cognitive function. In various embodiments, a patient or subject may be a human or any other animal (canine, feline, etc.).

In some embodiments, a method as provided herein involves the administration of a purified nadolol isomer to a subject desiring an improvement in cognition and/or a subject diagnosed with a neurodegenerative disease or disorder (such as described above); however, the purified nadolol isomer is not administered for the purpose of directly improving cognition and/or treating the neurological disease but rather for the purpose of countering side effects of a second agent that is administered to the subject to improve cognition and/or treat the neurological disease in the subject.

In some embodiments, the a purified nadolol isomer (NI-X) is administered in a dose of about 0.01 to 15 mg, 0.1 to 15 mg, 0.1 to 10 mg, 0.1 to 1 mg, 0.1 to 0.5 mg, 0.2 to 0.3 mg, 0.23 to 0.27 mg; 0.1 to 5 mg, 1 to 15 mg, 1 to 10 mg, 0.5-10 mg; 0.5-5 mg; 1 to 5 mg, 5 to 10 mg, 10 to 50 mg, 10 to 500 mg, 10 to 100 mg, 30 to 60 mg, 50 to 100 mg, 100 to 400 mg, 200 to 400 mg, 400 mg or less, 100 mg or less, 50 mg or less, 25 mg or less, 15 mg or less, 10 mg or less, 7 mg or less, 5 mg or less, 1 mg or less, about 0.01 mg, about 0.05 mg; about 0.1 mg, about 0.2 mg, about 0.25 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, about 10 mg, or about 20 mg, or about 25 mg, or about 50 mg, or about 75 mg, or about 100 mg, or about 125 mg, or about 150 mg, or about 175 mg, or about 200 mg, or about 250 mg, or about 300 mg, or about 320 mg. In some embodiments the aforementioned doses of a purified nadolol isomer (NI-X) is a total daily dose, a total weekly doses, or a total twice-weekly dose. In some embodiments, due to increased potency and/or gastric uptake, the dose of a purified nadolol isomer (NI-X) may be lower than that of a nadolol mixture, such as those currently traditionally commercially available.

In some aspects and embodiments, the present disclosure is further based at least in part on the finding that certain nadolol isomers have lower ability to cross the blood brain barrier or otherwise occupy receptors in the CNS as compared to other nadolol isomers. Accordingly, in certain embodiments of the compositions and methods provided herein, a purified nadolol isomer (NI-X) of the disclosure may have more consistent plasma exposure as compared to one or more other nadolol isomers and/or a nadolol racemic mixture, thus reducing the likelihood of needing dose adjustment due to food consumption or other drugs co-administered with nadolol. Accordingly, in certain embodiments, a purified nadolol isomer is preferred due to a decrease in its relative activity in the CNS as compared to the periphery (ie outside of the CNS). In some embodiments a preferred nadolol isomer is preferred due to improved properties with regard to receptor occupancy on beta-receptors in the CNS. In some embodiments, a purified nadolol isomer is preferred due to improved properties with regard to improved properties to CNS penetrability (eg., improved properties being lower CNS penetrability). In some embodiments, a purified nadolol isomer is preferred due to improved properties with regard to improved properties with regard to CNS transport out of the CNS (eg., improved properties being increased rate of transport out of the CNS). In some embodiments, a purified nadolol isomer is preferred due to improved properties with regard to improved properties to CNS penetrability (eg., improved properties being lower CNS penetrability and improved properties with regard to CNS transport out of the CNS (eg., improved properties being increased rate of transport out of the CNS). In some embodiments, a purified nadolol isomer is preferred due to improved properties with regard to receptor occupancy on beta-receptors in the CNS; wherein the improved CNS beta receptor occupancy properties are a result of improved CNS penetrability properties and/or improved CNS transport properties.

In various aspects and embodiments, a purified nadolol isomer formulation such as disclosed herein is used in combination with a second agent, or active ingredient. Accordingly, in one aspect provided is a method that involves administering a NI-X formulation and a second active ingredient to a subject. In one embodiment a NI-A formulation and a second active agent is administered to a subject. In one embodiment a NI-B formulation and a second active agent is administered to a subject. In one embodiment a NI-C formulation and a second active agent is administered to a subject. In one embodiment a NI-D formulation and a second active agent is administered to a subject. In one embodiment a NI-C/D formulation and a second active agent is administered to a subject. In another relevant aspect a NI-X formulation is provided that further includes a second active ingredient. In one embodiment a NI-A formulation is provided that further includes a second active ingredient. In one embodiment a NI-B formulation is provided that further includes a second active ingredient. In one embodiment a NI-C formulation is provided that further includes a second active ingredient. In one embodiment a NI-D formulation is provided that further includes a second active ingredient. In one embodiment a NI-C/D formulation is provided that further includes a second active ingredient. In some embodiments the second active ingredient is a β-AR agonist. In some embodiments the second active ingredient is a β₁-AR agonist. In some embodiments the second active ingredient is a β₂-AR agonist. In some embodiments the second active ingredient is a β agent.

In some embodiments a purified nadolol isomer formulation such as disclosed herein is used in combination with a β-AR agonist. In certain embodiments a purified nadolol isomer formulation such as disclosed herein (eg a NI-X formulation) is used in combination with a β-AR agonist, and the purified nadolol isomer (NI-X) is intended to offset undesirable side effects of the β-AR agonist. For example, in some embodiments a β-AR agonist is administered with the intent of acting on the CNS (such as administering a β-AR agonist for treating or improving cognitive function and/or treating a neurodegenerative disease) and the NI-X is administered to off-set or counter side effects of the β-AR agonist in the periphery. Accordingly, in one embodiment a NI-X formulation is provided that further includes a β-AR agonist. In one embodiment a NI-A formulation is provided that further includes a β-AR agonist. In one embodiment a NI-B formulation is provided that further includes a β-AR agonist. In one embodiment a NI-C formulation is provided that further includes a β-AR agonist. In one embodiment a NI-D formulation is provided that further includes a β-AR agonist. In one embodiment a NI-C/D formulation is provided that further includes β-AR agonist. In another aspect, provided is a method that includes administering a nadolol formulation (such as a purified nadolol formulation) as provided herein and a β-AR agonist to a subject. In one embodiment, provided is a method that includes administering a NI-D formulation as provided herein and a β-AR agonist to a subject. In one embodiment, provided is a method that includes administering a NI-C formulation as provided herein and a β-AR agonist to a subject. In one embodiment, provided is a method that includes administering a NI-B formulation as provided herein and a β-AR agonist to a subject. In one embodiment, provided is a method that includes administering a NI-A formulation as provided herein and a β-AR agonist to a subject.

As used herein, the term “β agonist” is used to mean an agent that acts as an agonist of a β-adrenergic receptor (β-AR). A β agonist may be a β₁ agonist, β₁ agonist, or a non-selective β agonist. In some embodiments, the β-AR agonist can be administered at a dose of from about 30 to 160 μg. In some embodiments, the β-AR agonist can be administered at a dose of from about 50 to 160 μg. For some embodiments, the β-AR agonist can be administered at a dose of from about 30 to 160 μg, 50 to 160 μg, 80 to 160 μg, 100 to 160 μg, 120 to 160 μg, 140 to 160 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, 30 μg, 40 μg, 50 μg, 60 μg, 70 μg, 80 μg, 90 μg, 100 μg, 110 μg, 120 μg, 130 μg, 140 μg, 150 μg, or 160 μg.

For some embodiments, the above-mentioned doses are a total daily dose. For some embodiments, the above-mentioned doses are a total weekly dose. For some embodiments, the dose of β-AR agonist is administered or weekly for a period of weeks or more. As used herein, the term “β₁ agonist” is used to mean β₁-adrenergic receptor agonist or β₁-AR agonist. In certain embodiments the term β₁ agonist is understood to include compounds that are primarily β₁ agonists, but which may also exhibit some peripheral agonism for other adrenergic receptors, such as β₂-adrenergic receptors. In this application, the terms “β₁-adrenergic receptor agonist”, “β₁-AR agonist”, “β₁ AR agonist” and “β₁ agonist” may be used interchangeably. In certain embodiments, the term β₁-AR agonist expressly includes both selective and partial agonists, as well as biased and non-biased agonists. Examples of β₁ adrenergic agonists include, for example, xamoterol, noradrenalin, isoprenaline, dopamine and dobutamine and the pharmaceutically-acceptable salts of any of the above. Partial agonists and ligands of the β₁-AR are known. Further, using the methodology of Kolb et al., but for β₁-AR instead, one skilled in the art could determine new ligands by structure-based discovery. See Proc. Natl. Acad. Sci. USA 2009, 106, 6843-648.

As used herein, the term “β₂ agonist” is used to mean β₂-adrenergic receptor agonist or β₂-AR agonist. In certain embodiments, the term β₂ agonist is understood to include compounds that are primarily β₂ agonists, but which may also exhibit some peripheral agonism for other adrenergic receptors, such as β₁-adrenergic receptors. In this application the terms “β₂-adrenergic receptor agonist”, “β₂-AR agonist”, “β₂AR agonist” and “β₂ agonist” may be used interchangeably. In some embodiments the term β₂-AR agonist expressly includes both selective and partial agonists. β₂ agonists that may be used in accordance with various aspects and embodiments of the present disclosure may be short-acting, long-acting or ultra long-acting. Examples of short-acting β₂ agonists that may be used are salbutamol, levosalbutamol, terbutaline, pirbuterol, procaterol, metaproterenol, bitolterol mesylate, oritodrine, isoprenaline, salmefamol, fenoterol, terbutaline, albuterol, and isoetharine. Examples of long-acting β₂ agonists that may be used are salmeterol, bambuterol, formoterol and clenbuterol. Examples of ultra long-acting β₂ agonists include indacaterol, vilanterol and olodaterol. Other examples of β₂ agonists include tulobuterol, mabuterol, and ritodrine. For some embodiments, the β₂-AR agonist can be administered at a dose of from 0.5-20 mg; or 1-10 mg; or 2-8 mg; or 1-500 mg; or 10-20 mg; or 20-50 mg; or 50-100 mg; or 1-100 mg; or 100-250 mg; or 250-500 mg; or about 1 mg; or about 2 mg; or about 3 mg; or about 4 mg; or about 5 mg; or about 6 mg; or about 7 mg; or about 8 mg; or about 10 mg.; or about 25 mg; or about 50 mg; or about 100 mg; or about 250 mg; or about 500 mg.

In some embodiments, the compositions and methods involve administering to a subject a purified nadolol isomer (NI-X) and clenbuterol. Clenbuterol is a β₂ agonist having the following chemical structure:

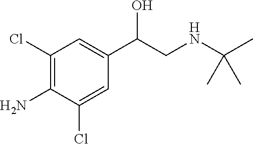 [SMILES: CC(C)(C)NCC(O)c1cc(Cl)c(N)c(Cl)c1]

In certain embodiments, clenbuterol as used herein refers to a racemic mixture. In other embodiments, the clenbuterol used herein may be (S)-clenbuterol that is substantially free of the (R)-clenbuterol isomer. In other embodiments, the clenbuterol used herein may be (R)-clenbuterol that is substantially free of the (S)-clenbuterol isomer. In some embodiments, clenbuterol can be administered at a dose of from about 30 to 160 μg. In some embodiments, clenbuterol can be administered at a dose of from about 50 to 160 μg or 80 to 160 μg. For some embodiments, the above-mentioned doses are a total daily dose. For some embodiments, the above-mentioned doses are a weekly dose. For some embodiments, the dose of clenbuterol and nadolol are administered for a period of weeks or more. For some embodiments, nadolol is a mixture of four isomers. For some embodiments, the nadolol administered is a specific enantiomerically pure isomer.

In some embodiments, the compositions and methods involve administering to a subject a purified nadolol isomer (NI-X) and tulobuterol. Tulobuterol is a long-acting β₂ agonist having the following chemical structure:

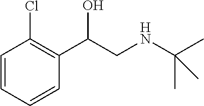

Tulobuterol is marketed in Japan as a racemic mixture for administration as a transdermal patch. In certain embodiments, tulobuterol as used herein refers to a racemic mixture. In other embodiments, the tulobuterol used herein may be (S)-tulobuterol that is substantially free of the (R)-tulobuterol isomer. In other embodiments, the tulobuterol used herein may be (R)-tulobuterol that is substantially free of the (S)-tulobuterol isomer. In some embodiments, tulobuterol can be administered in a dose from 0.5-20 mg; or 1-10 mg; or 2-8 mg; or about 1 mg; or about 2 mg; or about 3 mg; or about 4 mg; or about 5 mg; or about 6 mg; or about 7 mg; or about 8 mg; or about 10 mg. For some embodiments, the above-mentioned doses are a total daily dose. For some embodiments, the above-mentioned doses are a weekly dose. For some embodiments, the dose of tulobuterol is administered for a period of weeks or more.

In some embodiments the active ingredient or β agonist of the compositions and methods provided herein is a β-agent. The term “β-agent” as used herein means a compound with a structure of Formula (I), Formula (I″), Formula (II), Formula (III), Formula (I′), Formula (II′), Formula (III′), Formula (IV′), Formula (V′), Formula (VI′), Formula (VII′), Formula (VIII′), Formula (IX′), Formula (X′), Formula (XI′), Formula (XII′), Formula (XIII′), Formula (XIV′), Formula (XV′), Formula (XVI′), Formula (XVII′), Formula (XVIII′), Formula (XIX′), Formula (XX′), Formula (XXI′), Formula (XXII′), Formula (XXIII′), Formula (XXIV′), or Formula (XXV′) as provided herein; or an optically pure stereoisomer, pharmaceutically acceptable salt, solvate, or prodrug thereof in various embodiments, the β-agent is a compound provided in Table 1 herein. In some embodiments, the β-agent is Compound 03-5, or an optically pure stereoisomer, pharmaceutically acceptable salt, solvate, or prodrug thereof in certain embodiments, a β-agent as disclosed herein is an agonist, partial agonist or antagonist of an adrenergic receptor; in some embodiments the compound is a β1-adrenergic receptor agonist, β2-adrenertic receptor agonist or non-selective β1/β2-adrenergic receptor agonist; in some embodiments the compound is a β1-adrenergic receptor agonist; in some embodiments the compound is a β2-adrenergic receptor agonist; in some embodiments the compound is a compound is a non-selective β1/β2-adrenergic agonist.

In some embodiments a β-agent is a compound according to Formula (I) or an optically pure stereoisomer, pharmaceutically acceptable salt, solvate, prodrug thereof

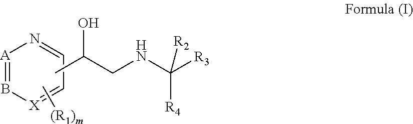

In some embodiments, each A, B, and X is independently a nitrogen or carbon. In some embodiments, each R₁ is independently selected from the group consisting of hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, and unsubstituted or substituted heteroaryl. In some embodiments, m is an integer selected from 0 to 4.

In some embodiments, R₂, R₃, and R₄ are independently selected from the group consisting of H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

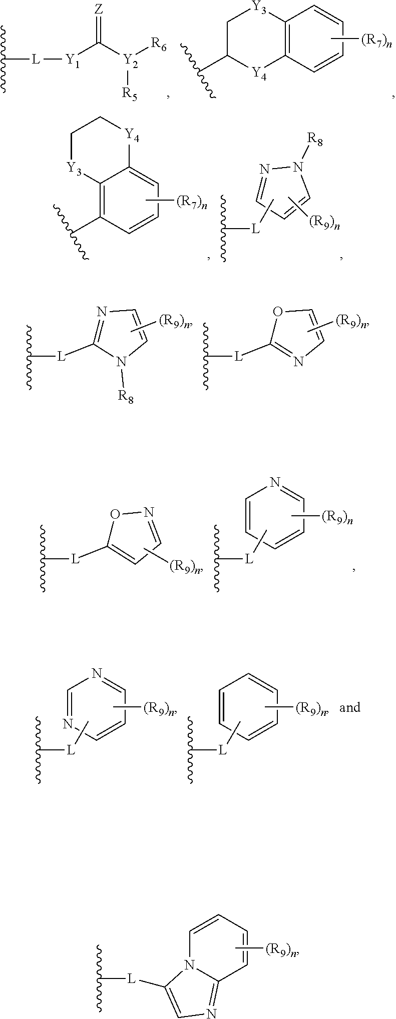

or R₂ and R₃ together with the carbon form an unsubstituted or substituted 3-7 membered cycloalkyl or heterocycle ring.

In some embodiments, L is a C1-C5 alkyl linker optionally substituted, each Y₁, Y₂, Y₃, and Y₄ is independently a covalent bond, a carbon, an oxygen, or a nitrogen, optionally substituted with hydrogen, unsubstituted or substituted alkyl, or unsubstituted or substituted cycloalkyl, and Z is O or S.

In some embodiments, R₅ and R₆ are independently selected from hydrogen, unsubstituted or substituted alkyl, or R₅ and R₆ are cyclically linked and together with Y₂ to form an optionally substituted cycloalkyl or heterocycle, each R₇ is independently selected from the group consisting of hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, and unsubstituted or substituted heteroaryl.

In some embodiments, n is an integer selected from 0 to 4, R₈ is selected from the group consisting of hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, and unsubstituted or substituted amino.

Also disclosed herein is a β-agent that is a compound according to Formula (II) or an optically pure stereoisomer, pharmaceutically acceptable salt, solvate, or prodrug thereof

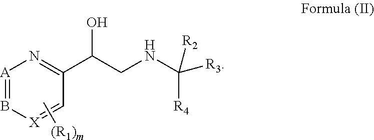

In some embodiments, each A, B, and X is independently a nitrogen or carbon. In some embodiments, each R₁ is independently selected from the group consisting of hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, and unsubstituted or substituted heteroaryl. In some embodiments, m is an integer selected from 0 to 4.

In some embodiments, R₂, R₃, and R₄ are independently selected from the group consisting of H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

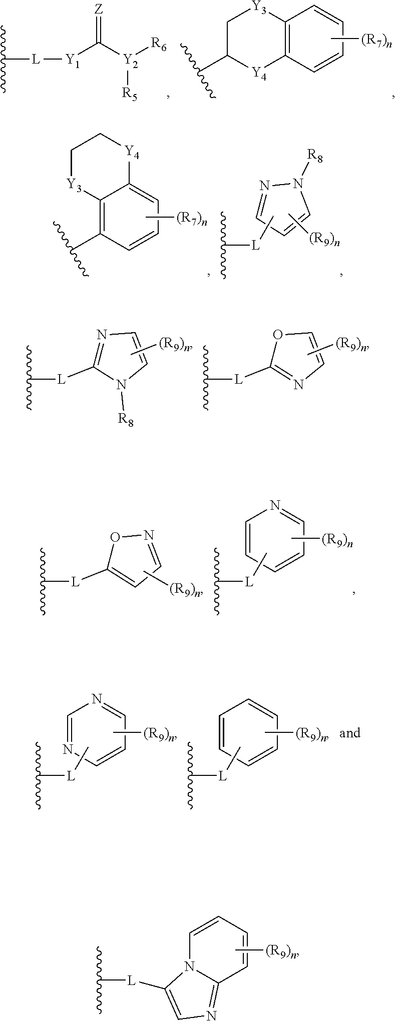

or R₂ and R₃ together with the carbon form an unsubstituted or substituted 3-7 membered cycloalkyl or heterocycle ring.

In some embodiments, L is a C1-C5 alkyl linker optionally substituted, each Y₁, Y₂, Y₃, and Y₄ is independently a covalent bond, a carbon, an oxygen, or a nitrogen, optionally substituted with hydrogen, unsubstituted or substituted alkyl, or unsubstituted or substituted cycloalkyl, and Z is O or S.

In some embodiments, R₅ and R₆ are independently selected from hydrogen, unsubstituted or substituted alkyl, or R₅ and R₆ are cyclically linked and together with Y₂ to form an optionally substituted cycloalkyl or heterocycle, each R₇ is independently selected from the group consisting of hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, and unsubstituted or substituted heteroaryl.

In some embodiments, n is an integer selected from 0 to 4, R₈ is selected from the group consisting of hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, and unsubstituted or substituted amino.

In further embodiments, a β-agent is a compound according to Formula (III) or an optically pure stereoisomer, pharmaceutically acceptable salt, solvate, or prodrug thereof

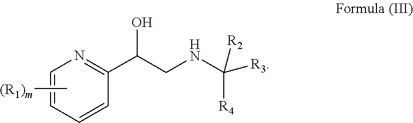

In some embodiments, each R₁ is independently selected from the group consisting of hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, and unsubstituted or substituted heteroaryl. m is an integer selected from 0 to 4.

In some embodiments, R₂, R₃, and R₄ are independently selected from the group consisting of H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

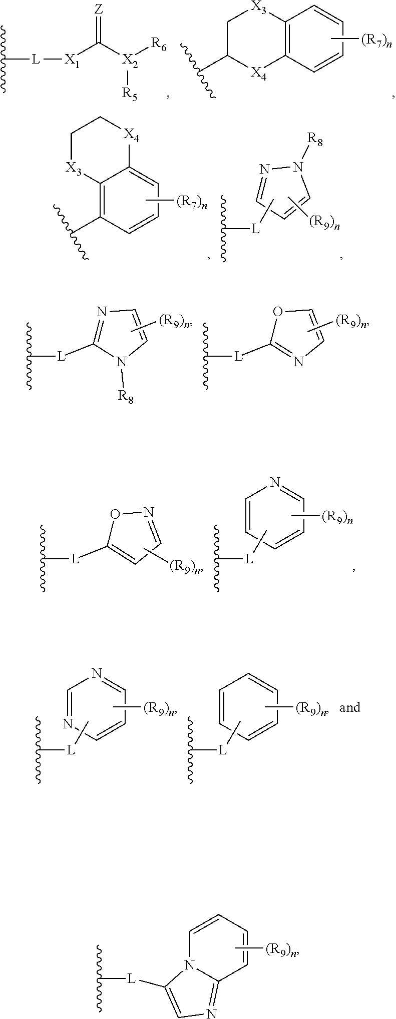

or R₂ and R₃ together with the carbon form an unsubstituted or substituted 3-7 membered cycloalkyl or heterocycle ring.

In some embodiments, L is a C1-C5 alkyl linker optionally substituted, each X₁, X₂, X₃, and X₄ is independently a covalent bond, a carbon, an oxygen, or a nitrogen, optionally substituted with hydrogen, unsubstituted or substituted alkyl, or unsubstituted or substituted cycloalkyl, and Y is O or S.

In some embodiments, R₅ and R₆ are independently selected from hydrogen, unsubstituted or substituted alkyl, or R₅ and R₆ are cyclically linked and together with Y₂ to form an optionally substituted cycloalkyl or heterocycle, each R₇ is independently selected from the group consisting of hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, and unsubstituted or substituted heteroaryl.

In some embodiments, n is an integer selected from 0 to 4, R₈ is selected from the group consisting of hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, and unsubstituted or substituted amino.

Further disclosed herein is a compound according to Formula (I′):

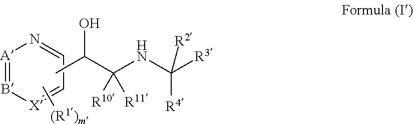

or a pharmaceutically acceptable salt thereof,
wherein:
   A′, B′, and X′ are each independently nitrogen or carbon;
   each R^{1′} is independently halogen, —R′, —CN, —NO₂, —SF₅, —OR^{x}, —NR^{x}₂, —NHR^{x}, —SO₂R′, —C(O)R′, —C(O)NR′₂;
   each R′ is independently hydrogen or an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
   each R^{x} is independently an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
   m′ is an integer selected from 0 to 4;
   R^{2′}, R^{3′}, and R^{4′} are each independently halogen, —R′, —CN, —NO₂, —OR′, —NR′₂,

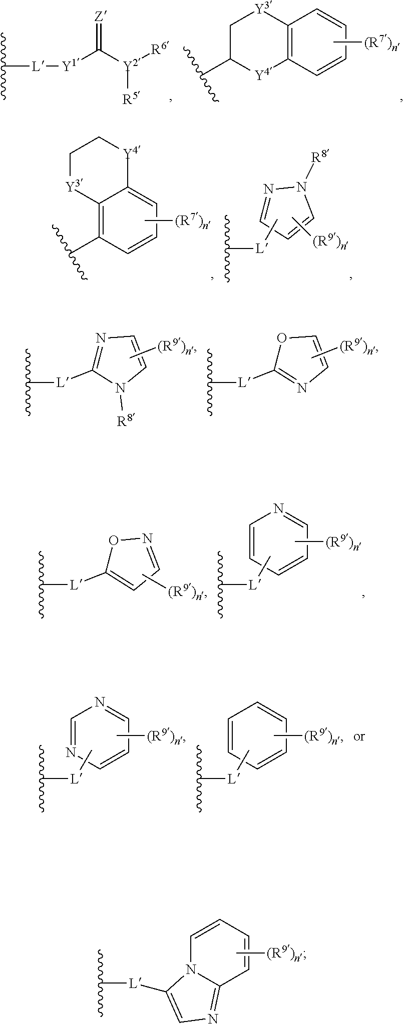

or
R²′ and R³′ together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;
L′ is optionally substituted C₁₋₅ alkylene;
Y^{1′}, Y^{2′}, Y^{3′}, and Y^{4′} are each independently a covalent bond, a carbon, an oxygen, or a nitrogen, optionally substituted with hydrogen, an optionally substituted C₁₋₆ alkyl, or an optionally substituted 3-7 membered saturated carbocyclic ring;
Z′ is O or S;
R^{5′} and R^{6′} are each independently hydrogen or optionally substituted alkyl, or
R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur;
each R^{7′} is independently —R′, halogen, —CN, —NO₂, —NR′₂, or —OR′;
n′ is an integer selected from 0 to 4;
R^{8′} is hydrogen, —CN, optionally substituted alkyl, or an optionally substituted aryl ring; and
each R^{9′} is independently hydrogen, halogen, —CN, —OR^{x}, —NR′₂, or optionally substituted alkyl; and
R^{10′} and R^{11′} are each independently hydrogen or optionally substituted C₁₋₂ aliphatic.

Further disclosed herein is a compound according to Formula (I″):

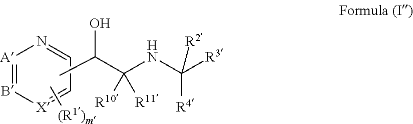

or a pharmaceutically acceptable salt thereof,
wherein:
   A′, B′, and X′ are each independently nitrogen or carbon;
   each R^{1′} is independently halogen, —R′, —CN, —NO₂, —SF₅, —OR^{x}, —NR^{x}₂, —NHR^{x}, —SO₂R′, —C(O)R′, —C(O)NR′₂, —NR′C(O)R′, —NR′CO₂R′, or —CO₂R′;
   each R′ is independently hydrogen or an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
   each R^{x} is independently an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
   m′ is an integer selected from 0 to 4;
   R^{2′}, R^{3′}, and R^{4′} are each independently halogen, —R′, —CN, —NO₂, —OR′, —NR′₂,

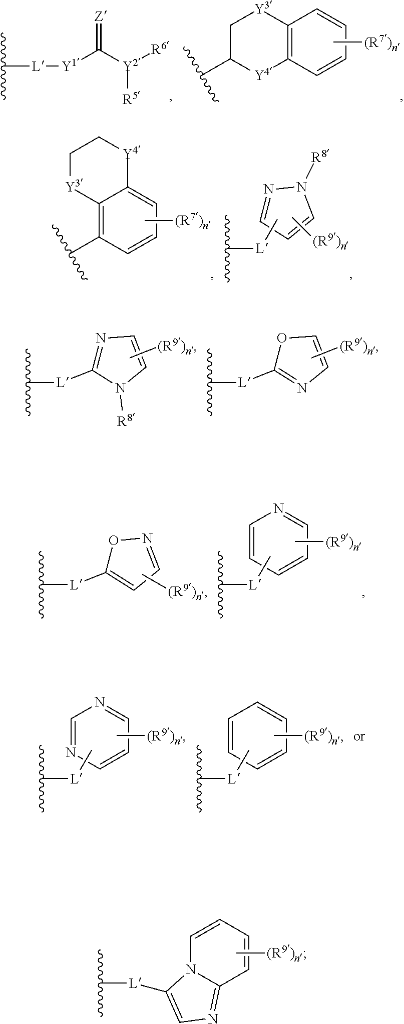

or
R²′ and R³′ together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;
L′ is optionally substituted C₁₋₅ alkylene;
Y^{1′}, Y^{2′}, Y^{3′}, and Y^{4′} are each independently a covalent bond, a carbon, an oxygen, or a nitrogen, optionally substituted with hydrogen, an optionally substituted C₁₋₆ alkyl, or an optionally substituted 3-7 membered saturated carbocyclic ring;
Z′ is O or S;
R^{5′} and R^{6′} are each independently hydrogen or optionally substituted alkyl, or
R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur;
each R^{7′} is independently —R′, halogen, —CN, —NO₂, —NR′₂, or —OR′;
n′ is an integer selected from 0 to 4;
R^{8′} is hydrogen, —CN, optionally substituted alkyl, or an optionally substituted aryl ring; and
each R^{9′} is independently hydrogen, halogen, —CN, —OR^{x}, —NR′₂, or optionally substituted alkyl; and
R^{10′} and R^{11′} are each independently hydrogen or optionally substituted C₁₋₂ aliphatic.

In some embodiments a β-agent is a compound with the following structure:

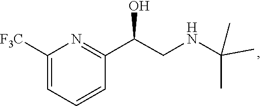 [SMILES: CC(C)(C)NC[C@H](O)c1cccc(C(F)(F)F)n1]

or a pharmaceutically acceptable salt thereof.

In some embodiments a β-agent is a compound with the following structure:

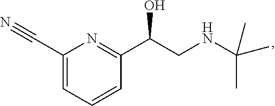 [SMILES: CC(C)(C)NC[C@H](O)c1cccc(C#N)n1]

or a pharmaceutically acceptable salt thereof.

In some embodiments a β-agent is a compound with the following structure:

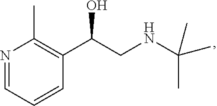

or a pharmaceutically acceptable salt thereof.

In certain embodiments a purified nadolol isomer formulation such as disclosed herein (eg a NI-X formulation) is administered to the patient prior to administration of β₁-AR agonist, a β₂-AR agonist, clenbuterol, and/or tulobuterol. In other embodiments, a purified nadolol isomer formulation such as disclosed herein (eg a NI-X formulation) is administered to the patient concurrently with the administration of a β₁-AR agonist, a β₂-AR agonist, clenbuterol, and/or tulobuterol. In other embodiments, a purified nadolol isomer formulation such as disclosed herein (eg a NI-X formulation) is co-administered to the patient in a single dosing formulation, in a single tablet and/or in a single capsule.

In certain embodiments of the compositions and methods provided herein, a purified nadolol isomer formulation such as disclosed herein (eg a NI-X formulation) is administered prior to or concurrently with a β₁-AR agonist, a β₂-AR agonist, clenbuterol, and/or tulobuterol in order to inhibit or preclude agonism of peripheral β₁ and/or β₂ adrenergic receptors by the β₁-AR agonist, β₂-AR agonist, clenbuterol, and/or tulobuterol. In various embodiments it is preferred to block peripheral β₁ and/or β₂ adrenergic receptors in accordance with the compositions and methods of the present disclosure in order to preclude, or at least minimize, any adverse effects, e.g., peripheral cardiac effects, on humans being treated.

In certain embodiments of the methods provided herein, the β₁-AR agonist, β₂-AR agonist, clenbuterol, and/or tulobuterol is administered orally, intravenously, intramuscularly, transdermally, by inhalation or intranasally. In certain embodiments of the methods provided herein, the β₁-AR agonist, β₂-AR agonist, clenbuterol, and/or tulobuterol is administered orally.

In certain embodiments of the methods provided herein, the a purified nadolol isomer formulation such as disclosed herein (eg a NI-X formulation) is administered orally, intravenously, intramuscularly, by inhalation or intranasally. In certain embodiments of the methods provided herein, the a purified nadolol isomer formulation such as disclosed herein (eg a NI-X formulation) is administered orally.

In certain embodiments of the methods provided herein, the β₁-AR agonist, β₂-AR agonist, clenbuterol, and/or tulobuterol and the a purified nadolol isomer formulation such as disclosed herein (eg a NI-X formulation) are administered to the patient in a single formulation. In some embodiments, the single formulation is in the form of a tablet. For some embodiments both agents (β-AR agonist and NI-X formulation) are present in a tablet. For some embodiments, the tablet includes 30 to 160 μg of clenbuterol, and/or 0.1 mg to 10 mg of tulobuterol, and from about 0.1 to 15 mg of the NI-X. For some embodiments, the tablet includes 30 to 160 μg of clenbuterol, and/or 0.1 mg to 10 mg of tulobuterol, and a NI-X in a subtherapeutic dose. For some embodiments, the tablet includes from about 0.5 to 20 mg of the β₁-AR agonist, β₂-AR agonist, clenbuterol, and/or tulobuterol, and from about 0.1 to 15 mg of the NI-X. In some embodiments, the tablet includes the peripherally acting β-blocker (PABRA) in a sub-therapeutic dose. In some embodiments, the tablet includes the NI-X in an amount that is 0.01 to 15 mg, 0.1 to 15 mg, 0.1 to 10 mg, 0.1 to 1 mg, 0.1 to 0.5 mg, 0.2 to 0.3 mg, 0.23 to 0.27 mg; 0.1 to 5 mg, 1 to 15 mg, 1 to 10 mg, 1 to 5 mg, 5 to 10 mg, 10 mg or less, 7 mg or less, 5 mg or less, 1 mg or less, about 0.01 mg, about 0.05 mg; about 0.1 mg, about 0.2 mg, about 0.25 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, or about 10 mg. For some embodiments the tablet having the aforementioned doses is administered daily. For some embodiments the tablet having the aforementioned doses is administered weekly.

In some embodiments, a method is provided that involves identifying a patient or subject desiring an improvement in cognition and/or identified as having a neurological disease or disorder and administering to the patient or subject a purified nadolol isomer formulation such as disclosed herein (eg a NI-X formulation) and a β-AR agonist.

In some embodiments of the methods and compositions provided herein, the purpose of the purified nadolol isomer (NI-X) is not to directly treat a specific disease indication or condition, but rather to offset undesirable peripheral side effects of a β-AR agonist (e.g., the purified nadolol isomer (NI-X) may be administered to reduce, restrict, or counter any adverse effect(s) of the β-AR agonist, such as cardiac effects or performance-enhancing effects, thus, reducing the likelihood of abuse), and therefore in some embodiments, the purified nadolol isomer (NI-X) dose may be lower than that generally used in previously approved therapeutic situations and indications where a beta-blocker is intended to directly treat a specific disease. In certain aspects a method for improving cognitive function and/or treating a neurodegenerative disease is provided wherein the method includes administering a therapeutically effective amount of β-AR agonist and a sub-therapeutic dose of purified nadolol isomer (NI-X) to a patient. As used herein, the term “sub-therapeutic dose” means a dose of an agent that is less than the minimum dose of the agent (including where the agent traditionally has been used in a racemic mixture) that is independently effective to treat a specific disease indication. In some embodiments, a sub-therapeutic dose is less than the lowest dose for which an agent (including where the agent traditionally has been used in a racemic mixture) is independently approved to treat any specific disease indication by a regulatory agency. In some embodiments, a sub-therapeutic dose is less than the lowest dose for which an agent is approved to treat any specific disease indication by the United States FDA. In some embodiments, a sub-therapeutic dose is less than the lowest dose for which an agent is approved to treat any specific disease indication by a regulatory agency (such as the US FDA). In certain embodiments, a subtherapeutic dose of a purified nadolol isomer (NI-X) is sufficient to off-set or counter one or more undesirable side effects of a β-AR agonist, but the dose is less than what would generally be administered to independently treat a disease or disorder. For example, in some embodiments a sub-therapeutic dose may be 90% or less; or 85% or less; or 80% or less; or 75% or less; or 70% or less; or 65% or less; or 60% or less; or 55% or less; or 50% or less; or 45% or less; or 40% or less; or 35% or less; or 30% or less; or 25% or less; or 20% or less; or 15% or less; or 10% or less; or 5% or less; or 4% or less; or 3% or less; or 2.5% or less; or 2% or less; or 1.5% or less; or 1% or less; or 0.5% or less as compared to a dose that the agent (including where the agent traditionally has been used in a racemic mixture) is effective for, or approved for treating a specific disease indication. In certain embodiments, a sub-therapeutic dose for a purified nadolol isomer (NI-X) may be about 90%; or about 85%; or about 80%; or about 75%; or about 70%; or 6 about 5%; or about 60%; or about 55%; or about 50%; or about 45%; or about 40%; or about 35%; or about 30%; or 25%; or about 20%; or about 15%; or about 10% or less; about 5%; or about 4%; or about 3%; or about 2.5%; or about 2%; or about 1.5% or less; or about 1%; or about 0.5% as compared to a dose that the agent (including where the agent traditionally has been used in a racemic mixture) is effective for, or approved for, treating a specific disease indication. For example, the nadolol (in a racemic mixture) at a dose of 40 mg once daily is approved in the United States for treatment of hypertension and angina pectoris, therefore a sub-therapeutic dose of a purified nadolol isomer (NI-X) in certain embodiments would be a dose that is less than 40 mg daily; for example a sub-therapeutic dose of a purified nadolol isomer (NI-X) may be 90% or less; or 85% or less; or 80% or less; or 75% or less; or 70% or less; or 65% or less; or 60% or less; or 55% or less; or 50% or less; or 45% or less; or 40% or less; or 35% or less; or 30% or less; or 25% or less; or 20% or less; or 15% or less; or 10% or less; or 5% or less; or 4% or less; or 3% or less; or 2.5% or less; or 2% or less; or 1.5% or less; or 1% or less; or 0.5% or less as compared to the 40 mg daily dose; or in some embodiments a sub-therapeutic dose of a purified nadolol isomer (NI-X) may be about 90%; or about 85%; or about 80%; or about 75%; or about 70%; or 6 about 5%; or about 60%; or about 55%; or about 50%; or about 45%; or about 40%; or about 35%; or about 30%; or 25%; or about 20%; or about 15%; or about 10% or less; about 5%; or about 4%; or about 3%; or about 2.5%; or about 2%; or about 1.5% or less; or about 1%; or about 0.5% of a 40 mg daily dose. In some embodiments, the a purified nadolol isomer (NI-X) is administered in a subtherapeutic dose of about 0.01 to 15 mg, 0.1 to 15 mg, 0.1 to 10 mg, 0.1 to 1 mg, 0.1 to 0.5 mg, 0.2 to 0.3 mg, 0.23 to 0.27 mg; 0.1 to 5 mg, 1 to 15 mg, 1 to 10 mg, 0.5-10 mg; 0.5-5 mg; 1 to 5 mg, 5 to 10 mg, 10 mg or less, 7 mg or less, 5 mg or less, 1 mg or less, about 0.01 mg, about 0.05 mg; about 0.1 mg, about 0.2 mg, about 0.25 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, or about 10 mg. In some embodiments the aforementioned doses of a purified nadolol isomer (NI-X) is a total daily dose, a total weekly doses, or a total twice-weekly dose.

Clenbuterol, and certain other β-agonists, have hypertrophic and lipolytic properties side effect that have resulted in illicit abuse by athletes and individuals desiring muscle building, athletic performance-enhancing, and/or weight loss. These side effects and propensity for abuse have created hurdles for regulatory approval (such as FDA approval) and create a certain level of a public health risk. However, the hypertrophic and lipolytic actions are caused in large part by activation of peripheral β receptors; accordingly the hypertrophic and lipolytic side effects and propensity for abuse can be reduced, mitigated or eliminated by co-administering a PABRA such as disclosed herein in combination with a β-agonist. In particular if the β-agonist and PABRA are made and sold only in single formulations having both agents such as described herein, then it will be very difficult or impossible for those seeking illicit use or abuse to separate the agents to make a product that would be effective for muscle building, athletic performance-enhancing, or weight loss illicit use. Accordingly, in some aspects and embodiments, provided are compositions and methods that involve a single formulation (such as, for example an oral tablet) having a β-agonist and PABRA, that is effective for improving cognition (a CNS action) but that have a reduced risk of illicit use/abuse as compared to a formulation having only a β-agonist without a PABRA. In many embodiments a sub-therapeutic dose of the PABRA is sufficient to counteract the side effects of the β-agonist, accordingly, a single formulation (such as, for example an oral tablet) as described herein having a β-agonist and PABRA may have a therapeutically active dose of the β-agonist and a sub-therapeutic dose of the PABRA.

The term “about” as used herein means in quantitative terms plus or minus 10%. For example, “about 3%” would encompass 2.7-3.3% and “about 10%” would encompass 9-11%. Moreover, where “about” is used herein in conjunction with a quantitative term it is understood that in addition to the value plus or minus 10%, the exact value of the quantitative term is also contemplated and described. For example, the term “about 3%” expressly contemplates, describes and includes exactly 3%.

“Therapeutically effective amount” as used herein means the amount of a compound or composition (such as described herein) that causes at least one desirable change in a cell, population of cells, tissue, individual, patient or the like. In some embodiments a therapeutically effective amount as used herein means the amount of a compound or composition (such as described herein) that prevents or provides a clinically significant change in a disease or condition (e.g., reduce by at least about 30 percent, at least about 50 percent, or at least about 90 percent) or in one or more features of a disease or condition described herein. In some embodiments, the term “therapeutically effective amount” means an amount of a compound or composition as described herein effective or sufficient to improve cognition and/or treat a neurodegenerative disease in a patient. The term “frequency” as related thereto means the number of times a treatment is administered to a patient in order to obtain the result of improved cognition and/or treating a neurodegenerative disease in a patient.

In certain embodiments, a patient or subject demonstrates improved cognition following administration of a NI-X formulation (for example in combination with a j-AR agonist) as described herein. In some embodiments, the patient demonstrates improved cognition as demonstrated by an improvement in a cognition test or model; a memory test; a diagnostic indicator of mental status, brain function, mental condition; a contextual learning test; brain imaging or the like in the patient. “Improving cognition,” “improved cognition” or “improvement in cognition” means an improvement in an individual's cognitive capacity, or memory, or the like. In certain embodiments, the methods described herein result in an improvement cognition, for example as demonstrated by an improvement in a cognition test, a memory test, brain imaging and/or a contextual learning test in the patient. In some embodiments, the methods described herein result in an improvement in a contextual learning test in the patient wherein said contextual learning test is a spatial contextual learning test or Arizona Cognitive Test Battery (ACTB).

In some embodiments, the patient or subject is a mammal. In some embodiments the patient or subject is a human. In some embodiments, the patient or subject is a child human. In some embodiments the patient or subject is an adult human. Child, as used herein, means a human from about 5 to 20 years of age. Adult, as used herein, means a human from about 21 years of age and older.

### DETAILED DESCRIPTION

The present invention is based, at least in part, on the finding that certain isomers of nadolol have improved properties over other isomers and/or a racemic mixture of nadolol isomers.

In one embodiment, the present invention provides a pharmaceutical composition suitable for reducing drug/drug interactions or food/drug interaction and can include

two or more pharmacologically active compounds (e.g., drugs), including but not limited to 1) a pure nadolol isomer that is not a substrate of gastric transporter OATP1A2; and 2) one or two more drugs co-administered.

In one embodiment of the invention, the active compounds are combined together in a single unit dose formulation and optionally include a pharmaceutically acceptable carrier. In another embodiment of the invention, the actives can be administered separately, sequentially or concomitantly.

In yet another embodiment, the present invention provides a pharmaceutical composition suitable for reducing food-drug interaction and/or drug-drug interaction resulting from the administration of nadolol comprising the (SSR)-nadolol and optionally a pharmaceutically acceptable carrier.

The methods provided herein (for example methods of improving cognition and/or treating a neurodegenerative disease or disorder) may include subjecting the patient to brain imaging to determine regional metabolic activation and/or cerebral perfusion in cerebrocortical, forebrain, midbrain and brainstem areas and/or to identify whether said patient is in need of or desiring improvement of cognitive function and/or treatment of a neurodegenerative disease. In some embodiments, the brain imaging is fluorodeoxyglucose positron emission tomography (FDG-PET), used alone or in combination with other imaging approaches such as magnetic resonance imaging (MRI) and CT. In some embodiments, the brain imaging is, or can include, magnetic resonance imaging-arterial spin labeling (MRI-ASL), or magnetic resonance imaging-blood oxygenation level dependent computerized tomography (MRI-BOLD). In some embodiments the brain imaging may include MRI-ASL used to monitor cerebral blood flow, including, for example, cerebral blood flow to the hippocampus or thalamus. In some embodiments, of the aspects and embodiments disclosed herein, “improving cognition and/or treating a neurodegenerative disease” in a patient may include improving cognitive and executive function, improving inflammatory status in cerebral or cerebrospinal fluid (CSF) samples, attenuating proteinopathy burden (for example, based on imaging or CSF sampling) and/or improving regional cerebral metabolic status (reversing hypometabolism) or perfusion in the patient. In certain embodiments of the methods and compositions disclosed herein a β-AR agonist is administered in a dose that is therapeutically effective in improving cognition and/or treating a neurodegenerative disease in a patient. As such, in certain embodiments, “identifying a patient in need of or desiring improvement of cognitive function and/or treatment of a neurodegenerative disease” may include identifying a patient in need of or desiring improvement of cognitive and executive function, improvement of inflammatory status in cerebral or CSF samples, attenuation of proteinopathy burden (for example, based on imaging or CSF sampling) and/or improvement of regional cerebral metabolic/perfusion status (reversing hypometabolism or hypoperfusion). In another aspect, a method is provided wherein the method includes subjecting a patient to brain imaging to determine regional metabolic activation or perfusion in cerebrocortical, forebrain, midbrain and brainstem areas and/or to identify whether said patient is in need of or desiring improvement of cognitive function and/or treatment of a neurodegenerative disease, and administering to said patient a β-AR agonist and a purified nadolol isomer (NI-X) to improve cognition and/or treat a neurodegenerative disease in said patient. For some embodiments, the purified nadolol isomer (NI-X) is administered to reduce, restrict, or counter any adverse effects of the β-AR agonist, e.g., performance-enhancing effects, and reduces the likelihood of abuse.

In certain aspects and embodiments of the present disclosure, compositions and methods result in an improved cognition, raised cerebral metabolic activity and/or improved inflammatory control in a patient. In some embodiments, the methods described herein result in an improvement cognition, for example as demonstrated by an improvement in a cognition test or model; a memory test; a diagnostic indicator of mental status, brain function, mental condition; a contextual learning test; or the like in the patient. Such cognitive tests, diagnostics and models are well known in the art. In various aspects and embodiments, any of many accepted contextual learning tests for animals or humans can be used to assess baseline cognitive function and/or to measure or quantify improved cognitive function. In some embodiments, the compositions and methods described herein may result in an improvement one or more tests, diagnostics and models as follows. Likewise, for the raised cerebral metabolic activity and improved inflammatory control—these in certain embodiments may be imaged via FDG-PET and via sampling of cerebrospinal fluid (CSF) allowing measures of inflammatory cytokines and markers of glial cell activation. In some embodiments, magnetic resonance imaging-arterial spin labeling (MRI-ASL) can be used for neuroimaging. In some embodiments, magnetic resonance imaging-blood oxygenation level dependent computerized tomography (MRI-BOLD) can be used for neuroimaging. In various embodiments, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

### Human Models/Tests

There are many contextual learning tests used that are acknowledged and/or accepted in the art that in various embodiments may be used in conjunction with the compositions and methods disclosed herein to assess baseline cognitive function and/or to measure or quantify improved cognitive function in human subjects. For example, the contextual learning test used may be based upon single task learning, multiple task learning or spatial contextual memory. Contextual learning test evaluations based upon spatial contextual memory may be advantageous in assessing, for example, how well an individual is able to navigate a shopping mall, his or her neighborhood or a city transit or subway system as well as assessing any improvements in the ability to execute these tasks resulting from the treatment methods described herein.

An example of a simple spatial contextual learning test is contextual cuing, where humans learn to use repeated spatial configurations to facilitate a target search. A higher order spatial contextual learning test is serial learning, where humans learn to use subtle sequence regularities to respond more quickly and accurately to a series of events. See, for example, J. H. Howard Jr., et al., Neuropsychology, Vol. 18(1), January 2004, 124-134.

In some embodiments, cognition may be evaluated using the Mini-Mental State Examination (MMSE) and/or the Montreal Cognitive Assessment (MOCA).

Arizona Cognitive Test Battery (ACTB). A testing protocol that may be used in various embodiments is the Arizona Cognitive Test Battery (ACTB). See Edgin, J., et al. J. Neurodevelop. Disord. (2010) 2: 149-164. The ACTB has been developed specifically to assess the cognitive phenotype in DS and includes various tests with various task demands and links with brain function. In more detail, tests are included for: 1) benchmarks, such as KBIT II verbal subscale and KBIT II non-verbal subscale IQ tests, 2) hippocampal function, 3) prefrontal function, 4) cerebellar function, 5) Finger sequencing tasks, 6) NEPSY visuomotor precision and 7) simple reaction time.

In some embodiments, cognition may be evaluated using the Cambridge Neuropsychological Test Automated Battery (CANTAB) assessment (see, for example, Sahakian, et al., (1988). Brain. 111 (3): 695-718). Cognitive domains, such as attention, visuospatial working memory, episodic memory, speed of process and executive function can be assessed using the CANTAB Battery Test, which includes the following:
Reaction Time (RTI),
Paired Associates Learning (PAL),
Verbal Recognition Memory (VRM) Immediate Free Recall,
Rapid Visual Information Processing (RVP),
Spatial Working Memory (SWM),
Adaptive Tracking, and
VRB Delayed Free recall and Forced-Choice Recognition.

A correlation of domain/test, test description and certain primary abilities assessed in accordance with the ACTB is provided below:

| | | Primary Ability |
|---|---|---|
| Domain/Test | Description | Assessed |
| 1) Benchmark | Points to pictures based | Verbal |
| KBIT-II verbal subscale | on word or phrase | comprehension |
| KBIT-II nonverbal | Semantic or visuo-spatial | Problem solving |
| subscale | pattern completion | |
| 2) CANTAB | Touching boxes in | Immediate memory |
| spatial span | order of changing color | for spatial-temporal |
| | on screen | sequence |
| 3) Prefrontal | Press button below a cat, | Inhibitory control |
| Modified dots task | shifts to new rule, press | working memory |
| | across screen for | |
| | a frog, etc. | |
| 4) CANTAB IED | Forced-choice | Set-shifting |
| | discrimination task | |
| | with change in relevant | |
| | dimension | |
| 5) Hippocampal | Recall for hidden | Spatial associative |
| CANTAB | abstract patterns | memory |
| paired associates | | |
| 6) Virtual computer- | Navigation of a virtual | Spatial memory |
| generated arena | arena(via joystick) to | |
| | find a hidden target | |
| 7) Cerebellar | Sequences generated by | Motor sequencing |
| Finger-sequencing | tapping a number of | |
| task | fingers (1, 2, 3, 4) to a | |
| | lever in succession | |
| 8) NEPSY visuo-motor | Follows two tracks | Visuo-motor |
| precision | with a pen | tracking, |
| | | hand-eye coord. |
| 9) CANTAB simple | Participants | Motor response |
| reaction time | press button in | time and |
| | response to a box | attention |
| | presented on a | |
| | screen | |

The above battery of tests in some embodiments may all be performed in order to assess all major cognitive processes balanced by the practical need for testing under time constraints. The cognitive tests herein may in certain embodiments be used in patients receiving treatment herein to monitor the patient's cognitive status and progression.

In some embodiments, the battery of tests may be conducted with a test group of individuals, and a control group of individuals to demonstrate the effectiveness of various aspects and embodiments of the compositions and methods described herein. The test group may be treated with any of the treatment regimens described herein, and the control group is treated with placebo, such as a dextrose 5% saline solution by intranasal administration.

An improvement in cognitive function as defined herein as being at least a 10%, and preferably at least a 20% score improvement, on at least one, and preferably two or more, of the tests listed in the ATCB, for example. Anyone of the domain/tests listed for the ATCB above may be included in assessing whether an improvement occurred. Testing may be conducted after treatment or during treatment to ascertain whether modifications in dosage or frequency of treatment is warranted.

Brain Imaging. Generally, any non-invasive procedure many be used to both establish a baseline of brain pathology (existent or non-existent) from which baseline a treatment protocol is established. However, magnetic resonance imaging (MRI) may in some embodiments be preferred for neuroimaging examination because it allows for accurate measurement of the 3-dimensional (3D) volume of brain structures, especially the hippocampus and related regions. Such techniques are well known as described in U.S. Pat. No. 6,490,472, which patent is incorporated herein in the entirety.

Moreover, non-invasive optical imaging systems may also be used for monitoring neurological pathological events. See, for example, U.S. patent publication 2011/0286932, which is incorporated herein in the entirety. The technique described therein entails administration of a fluorescent marker to a human for staining Aβ peptides, imaging the retina of the DS human with an optical imaging system, and examining the images for stained Aβ peptides in order to determine whether onset of brain pathology (such as AD brain pathology) has occurred.

In certain embodiments, fluorodeoxyglucose positron emission tomography (FDG-PET) may be used for neuroimaging to determine cognitive function and/or identify a neurodegenerative disease in accordance with the compositions and methods described herein. The use of FDG-PET for monitoring cognitive function and/or diagnosing cognitive impairments or neurodegenerative diseases, and/or identifying patients in need of or desiring a treatment to improve cognitive function is described in, for example Brown et al., RadioGraphics, (2014) 34:684-701, and Shivamurthy et al., AJR, (2015) 204:W76-W85; both hereby incorporated by reference in their entirety. In various embodiments, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

### Alzheimer's Disease

AD brain pathology refers to the accumulation of highly degradation-resistant amyloid fibers that cause lesions in areas of the brain proximate thereto. Accumulation of these amyloid fibers to neurotoxic levels leads to destruction of nerve fibers, which, in turn, leads to the observed behavior associated with Alzheimer's dementia. Observed behavioral symptoms, which become progressively more severe with progression of the disease, often include loss of vocabulary, incorrect word substitutions (paraphasias), loss of reading and writing skills, increased risk of falling, wandering, loss of speech, apathy and even loss of muscle mass.

### Down Syndrome

Creation of several trisomic mouse models has greatly facilitated progress in the understanding the neurobiological basis of cognitive dysfunction in DS. Among the mouse models, the Ts65Dn mouse is best characterized. It has an extra copy of approximately 140 mouse genes on chromosome 16, orthologous to those on human chromosome 21 (HSA21). Almost all genes in HSA21 with potential role in nervous system abnormalities are also found in Ts65Dn mice. Similar to DS, alterations in the structure and function of the hippocampus and failure in the induction of long-term potentiation (LTP) have been extensively reported in Ts65Dn mice. Ts65Dn mice are the most widely used in DS research and are considered to be an art-accepted model for investigations regarding DS in humans. Olson, L. E., et al., Dev. Dyn. 2004 July; 230(3):581-9.

DS is characterized by degeneration and dysfunction of multiple neuronal populations in the central nervous system (CNS). Among them, the hippocampal formation, i.e. the primary site for processing contextual learning shows significant abnormalities in DS. As a result, failure in contextual learning is a common finding in people with DS. To uncover the neurobiological basis of failed contextual learning in DS, the integrity of subcortical regions extensively projecting to the hippocampal formation have been examined. Through extensive innervation, these subcortical regions impose strong modulatory influence on hippocampal neurons. Among these subcortical regions, LC is of particular importance. LC neurons in the brainstem are the sole supplier of massive norepinephrine (NE)-ergic terminals for the hippocampus and play a significant role in wakefulness, attention, and navigational memory. Significant age-related degeneration of NE-ergic neurons of LC in Ts65Dn mice was found. Interestingly, the loss of LC terminals in Ts65Dn mice leads to further deterioration of cognitive dysfunction in these mice. Similarly, LC neurons undergo extensive age-dependent degeneration in DS. The critical role of NE-ergic system dysfunction in cognitive dysfunction in Ts65Dn has been supported by the fact that increasing brain NE levels with L-threo-3, 4-dihydroxyphenylserine (L-DOPS), i.e. a NE prodrug, restored contextual learning in Ts65Dn mice. Although L-DOPS is in phase III clinical trial for the treatment of primary autonomic failure associated with Parkinson's disease, it is yet to be approved by the FDA and its long-term effects particularly in children have yet to be explored.

With respect to the agents described herein, the terms “modulate” and “modulation” refers to the upregulation (i.e., activation or stimulation) or downregulation (i.e., inhibition or suppression) of a response. A “modulator” is an agent, compound, or molecule that modulates, and may be, for example, an agonist, antagonist, activator, stimulator, suppressor, or inhibitor. The terms “inhibit”, “reduce”, remove as used herein refer to any inhibition, reduction, decrease, suppression, downregulation, or prevention in expression, activity or symptom and include partial or complete inhibition of activity or symptom. Partial inhibition can imply a level of expression, activity or symptom that is, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression, activity or symptom. The terms “eliminate” or “eradicate” indicate a complete reduction of activity or symptom.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

Two basic techniques are widely used for nuclear imaging: positron emission tomography (PET) and single photon emission computed tomography (SPECT). PET detects photons generated through positron-electron annihilation of positrons from a diagnostic radiopharmaceutical tracer placed in the subject, e.g., patient, to be imaged, and analyzes the photon energy and trajectory to generate tomographic images of the patient. SPECT generates images by computer analysis of photon emission events from a diagnostic radiopharmaceutical tracer having gamma emitting isotopes. Both PET and SPECT require the detection and analysis of single photon events, which are characterized by low signal to noise ratio and scarcity relative to the background radiation. Other constraints on the PET and SPECT image qualities include the sensitivity, temporal and spatial resolution, dynamic range, response time and counting rate characteristics of the data acquisition probe devices, e.g., photomultipliers and the like.

Radioisotopes that emit both high energy γ and/or low energy 7, p and/or positron radiation and which can be used per se or as a part of a compound as radiopharmaceuticals, include, without limitation, technetium-99m (^{99m}Tc), gallium-67 (⁶⁷Ga), thallium-201 (²⁰¹Tl), 111indium-(¹¹¹In), iodine-123 (¹²³I), iodine-125 (¹²⁵I), iodine-131 (¹³¹I), xenon-133 (¹³³Xe), and fluorine-18 (¹⁸F). All these isotopes, except ^{99m}Tc, ¹³¹I and ¹³³Xe, are produced in particle accelerators.

Non-limiting examples of commonly used radiotracers include ^{99m}Tc-Arcitumomab (CEA-Scan™) which is a monoclonal antibody for imaging colorectal tissues afflicted with colorectal cancer, ^{99m}Tc-sestamibi (Cardiolite™) and ^{99m}Tc-tetrofosmin (Myoview™) for imaging the heart of a subject for myocardial perfusion, ¹¹¹In-Capromab pendetide (ProstaScint™) which is a monoclonal antibody for imaging prostate tissues afflicted with prostate cancer, ^{99m}Tc-Fanolesomab (NeutroSpec™) which is a monoclonal antibody for imaging inflamed and infectious tissues and ⁹⁰Y/111In-Zevalin (Ibritumomab Tiuxetan) which is a monoclonal antibody directed against the CD20 antigen, whereby this antigen is found on the surface of normal and malignant B lymphocytes.

Any diagnostic radiopharmaceutical can be utilized in the kit of the present embodiments. Exemplary radiopharmaceuticals that can be utilized in this context of the present invention include, without limitation, ³H-water, ³H-inulin, ¹¹C-carbonmonoxide, ¹³N-ammonia, ¹⁴C-inulin, ¹⁵O—H₂O, ¹⁵O—O₂, ¹⁸F-fluorodeoxyglucose, ¹⁸F-sodium fluoride, ⁵¹Cr-erythrocytes (RBC), ⁵⁷Co-vitamin B12 (cyanocobalamin), ⁵⁸Co-vitamin B12 (cyanocobalamin), ⁵⁹Fe-citrate, ⁶⁰Co-vitamin B12 (cyanocobalamin), ⁶⁷Ga-citrate, ⁶⁸Ga-citrate, ⁷⁵Se-selenomethionine, ^{81m}Kr-krypton for inhalation, oral administration or injections, ⁸²Rb, ⁸⁵Sr-nitrate, ⁹⁰Y/¹¹¹In-ibritumomab tiuxetan (⁹⁰Y/¹¹¹In-Zevalin), ^{99m}Tc-albumin microspheres, ^{99m} Tc-disofenin, lidofenin and mebrofenin, ⁹⁹mTc-DMSA, ^{99m}Tc-DTPA (injection), ^{99m}Tc-DTPA (aerosol), ^{99m}Tc-ECD (ethylene cystate dimer), ^{99m}Tc-exametazime (HMPAO), ^{99m}Tc-glucoheptonate, ^{99m}Tc-HEDP, ^{99m}Tc-HMDP, ^{99m}Tc-HSA, ^{99m}Tc-MAA, ^{99m}Tc-MAG.sub.3, ^{99m}Tc-MDP, ^{99m}Tc-tetrofosmin (Myoview), ^{99m}Tc-sestamibi (Cardiolite), ^{99m}Tc-oral administrations, ^{99m}Tc-pertechnetate, ^{99m}Tc-pyrophosphate, ^{99m}Tc-RBC in vitro and in vivo labeling, ^{99m}Tc-sulfur colloid, ^{99m}Tc-teboroxime, ^{99m}Tc-white blood cells, ¹¹¹In-ibritumomab tiuxetan (¹¹¹In-Zevalin), ¹¹¹In-DTPA, ¹¹¹In-platelets, ¹¹¹ In-RBC, ¹¹¹In-white blood cells, ¹²³I-hippuran, ¹²³I-IMP, ¹²³I-mIBG, ¹²³I-sodium iodide, ¹²⁴I-sodium iodide, ¹²⁵I-fibrinogen, ¹²⁵I-IMP, ¹²⁵I-mIBG, ¹²⁵I-sodium iodide, ¹²⁶I-sodium iodide, ¹³⁰1-sodium iodide, ¹³¹I-hippuran, ¹³¹I-HSA, ¹³¹I-MAA, ¹³¹I-mIBG, ¹³¹I-Rose Bengal, ¹³¹I-sodium iodide, ¹²⁷Xe-inhalation and injection, ¹³³Xe-inhalation and injection, ¹⁹⁷Hg-chlormerodrin, ¹⁹⁸Au-colloid and ²⁰¹Tl-chloride.

The diagnostic methods described herein may also but utilized to assess the effectiveness of a particular therapeutic regimen. For example, a patient that has been identified as being in need of or desiring improvement of cognitive function and/or treatment of a neurodegenerative disease and which is being treated, may be diagnosed or otherwise assessed to determine the effectiveness of the treatment regime. While the diagnosis or assessment may be performed by any method known in the art, cognitive testing or brain imaging may be used to determine improvement of cognitive function or amelioration of a disease. In embodiments, cognitive testing or brain imaging may be used alone or in combination. In embodiments where brain imaging is utilized, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

The assessment of treatment efficacy may be utilized to alter the treatment regime of a patient. For example, the assessment may be utilized to alter dosing, timing of administration, and/or the actives of the pharmaceutical composition. In embodiments, the dosage of a particular pharmaceutical agent being administered to the patient may be lowered by combining administration with a different agent. In this manner, treatment may be optimized by altering the pharmaceutical composition to include different combinations of β₁-AR agonist, β₂-AR agonist, and peripherally acting β-blocker (PABRA). Dosing may also be altered depending on the timing of administration. For example, a shorter duration between each administration of the pharmaceutical composition may require a lower dose of active agent, while a longer duration between each administration of the pharmaceutical composition may require a higher dose of active agent, either of which may improve the treatment regime as determined by diagnosis or assessment of the patient.

In one embodiment, a patient may be assessed a single time during the course of treatment to optimize the treatment regime. Alternatively, the patient may be assessed multiple times over the course of treatment to continually optimize the treatment regime as directed by a medical professional.

### Dosage, Administration and Pharmaceutical Formulation

The term “pharmaceutically-accepted salts” means acid addition salts that are commonly used in human or veterinary medicine and are deemed safe for use. Examples for the present disclosure include, but are not limited to, salts obtained from the following acids: acetic, ascorbic, benzenesulfonic, benzoic, camphosulfonic, citric, ethanesulfonic, edisylic, fumaric, gentisic, gluconic, glucoronic, glutamic, hippuric, hydrobromic, isethionic, lactic, nitric, phosphoric, succinic, sulfuric and tartaric, for example. Any hydrated forms of such salts are also included in this definition. Thus, for example, both fumarate and hemifumarate salts are specifically contemplated as well as any hydrates thereof. For example, fumarate dihydrate may be specifically mentioned.

The pharmaceutical preparation in some embodiments may be in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. Preferably, the unit dosage form is a tablet. The composition can, if desired, also contain other compatible therapeutic agents. Preferred pharmaceutical preparations can deliver the compounds of the disclosure in a sustained release formulation.

For a binding agent, composition, or compound according to the present disclosure, the dosage form may optionally be a liquid dosage form. Solutions can be prepared in water suitably mixed with a surfactant such as hydroxypropylcellulose or an emulsifier such as polysorbate. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. Formulations optionally contain excipients including, but not limited to, a buffering agents, an anti-oxidant, a stabilizer, a carrier, a diluent, and an agent for pH adjustment. The pharmaceutical forms suitable for injectable use include sterile aqueous solutions or dispersion and sterile powders for the extemporaneous preparation of sterile injectable solutions or dispersions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum, albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS or polyethylene glycol (PEG).

In certain embodiments, an agent in accordance with the methods provided herein is administered orally, subcutaneously (s.c.), intravenously (i.v.), intramuscularly (i.m.), intranasally or topically. Administration of an agent described herein can, independently, be one to four times daily; or one or two times weekly; or one to four times per month; or one to six times per year or once every two, three, four or five years. Administration can be for the duration of one day or one month, two months, three months, six months, one year, two years, three years, and may even be for the life of the human patient. The dosage may be administered as a single dose or divided into multiple doses. In some embodiments, an agent is administered about 1 to about 3 times (e.g. 1, or 2 or 3 times).

### β-Agents

Alkyl groups refer to univalent groups derived from alkanes by removal of a hydrogen atom from any carbon atom, which include straight chain and branched chain with from 1 to 12 carbon atoms, and typically from 1 to about 10 carbons or in some embodiments, from 1 to about 6 carbon atoms, or in other embodiments having 1, 2, 3 or 4 carbon atoms. Examples of straight chain alkyl groups include, but are not limited to, methyl, ethyl, n-propyl, n-butyl, n-pentyl, and n-hexyl groups. Examples of branched chain alkyl groups include, but are not limited to isopropyl, isobutyl, sec-butyl and tert-butyl groups. Alkyl groups may be substituted or unsubstituted. Representative substituted alkyl groups may be mono-substituted or substituted more than once, such as, but not limited to, mono-, di-, or tri-substituted. As used herein, the term alkyl, unless otherwise stated, refers to both cyclic and noncyclic groups.

The terms “cyclic alkyl” or “cycloalkyl” refer to univalent groups derived from cycloalkanes by removal of a hydrogen atom from a ring carbon atom. Cycloalkyl groups are saturated or partially saturated non-aromatic structures with a single ring or multiple rings including isolated, fused, bridged, and spiro ring systems, having 3 to 14 carbon atoms, or in some embodiments, from 3 to 12, or 3 to 10, or 3 to 8, or 3, 4, 5, 6 or 7 carbon atoms. Cycloalkyl groups may be substituted or unsubstituted. Representative substituted cycloalkyl groups may be mono-substituted or substituted more than once, such as, but not limited to, mono-, di-, or tri-substituted. Examples of monocyclic cycloalkyl groups include, but are not limited to cyclopropyl, cyclobutyl, cyclopentyl, and cyclohexyl groups. Examples of multi-cyclic ring systems include, but are not limited to, bicycle[4.4.0]decane, bicycle[2.2.1]heptane, spiro[2.2]pentane, and the like. (Cycloalkyl)oxy refers to —O— cycloalkyl. (Cycloalkyl)thio refers to —S-cycloalkyl. This term also encompasses oxidized forms of sulfur, such as —S(O)-cycloalkyl, or —S(O)₂-cycloalkyl.

Alkenyl groups refer to straight and branched chain and cycloalkyl groups as defined above, with one or more double bonds between two carbon atoms. Alkenyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkenyl groups may be substituted or unsubstituted. Representative substituted alkenyl groups may be mono-substituted or substituted more than once, such as, but not limited to, mono-, di-, or tri-substituted. Examples of alkenyl groups include, but are not limited to, vinyl, allyl, —CH═CH(CH₃), —CH═C(CH₃)₂, —C(CH₃)═CH₂, cyclopentenyl, cyclohexenyl, butadienyl, pentadienyl, and hexadienyl, among others.

Alkynyl groups refer to straight and branched chain and cycloalkyl groups as defined above, with one or more triple bonds between two carbon atoms. Alkynyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkynyl groups may be substituted or unsubstituted. Representative substituted alkynyl groups may be mono-substituted or substituted more than once, such as, but not limited to, mono-, di-, or tri-substituted. Exemplary alkynyl groups include, but are not limited to, ethynyl, propargyl, and —C≡C(CH₃), among others.

Aryl groups are cyclic aromatic hydrocarbons that include single and multiple ring compounds, including multiple ring compounds that contain separate and/or fused aryl groups. Aryl groups may contain from 6 to about 18 ring carbons, or in some embodiments from 6 to 14 ring carbons or even 6 to 10 ring carbons in other embodiments. Aryl group also includes heteroaryl groups, which are aromatic ring compounds containing 5 or more ring members, one or more ring carbon atoms of which are replaced with heteroatom such as, but not limited to, N, O, and S. Aryl groups may be substituted or unsubstituted. Representative substituted aryl groups may be mono-substituted or substituted more than once, such as, but not limited to, mono-, di-, or tri-substituted. Aryl groups include, but are not limited to, phenyl, biphenylenyl, triphenylenyl, naphthyl, anthryl, and pyrenyl groups. Aryloxy refers to —O-aryl. Arylthio refers to —S-aryl, wherein aryl is as defined herein. This term also encompasses oxidized forms of sulfur, such as —S(O)-aryl, or —S(O)₂-aryl. Heteroaryloxy refers to —O-heteroaryl. Heteroarylthio refers to —S-heteroaryl. This term also encompasses oxidized forms of sulfur, such as —S(O)-heteroaryl, or —S(O)₂-heteoaryl.

Suitable heterocyclyl groups include cyclic groups with atoms of at least two different elements as members of its rings, of which one or more is a heteroatom such as, but not limited to, N, O, or S. Heterocyclyl groups may include 3 to about 20 ring members, or 3 to 18 in some embodiments, or about 3 to 15, 3 to 12, 3 to 10, or 3 to 6 ring members. The ring systems in heterocyclyl groups may be unsaturated, partially saturated, and/or saturated. Heterocyclyl groups may be substituted or unsubstituted. Representative substituted heterocyclyl groups may be mono-substituted or substituted more than once, such as, but not limited to, mono-, di-, or tri-substituted. Exemplary heterocyclyl groups include, but are not limited to, pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, aziridinyl, imidazolidinyl, pyrazolidinyl, thiazolidinyl, tetrahydrothiophenyl, tetrahydrofuranyl, dioxolyl, furanyl, thiophenyl, pyrrolyl, imidazolyl, pyrazolyl, pyrazolinyl, triazolyl, tetrazolyl, oxazolyl, isoxazolyl, thiazolyl, thiazolinyl, oxetanyl, thietanyl, homopiperidyl, oxepanyl, thiepanyl, oxazepinyl, diazepinyl, thiazepinyl, 1,2,3,6-tetrahydropyridyl, indolinyl, 2H-pyranyl, 4H-pyranyl, dioxolanyl, dioxanyl, purinyl, quinolizinyl, cinnolinyl, phthalazinyl, pteridinyl, and benzothiazolyl groups. Heterocyclyloxy refers to —O-heterocycyl. Heterocyclylthio refers to —S-heterocycyl. This term also encompasses oxidized forms of sulfur, such as —S(O)-heterocyclyl, or —S(O)₂-heterocyclyl.

Polycyclic or polycyclyl groups refer to two or more rings in which two or more carbons are common to the two adjoining rings, wherein the rings are “fused rings”; if the rings are joined by one common carbon atom, these are “spiro” ring systems. Rings that are joined through non-adjacent atoms are “bridged” rings. Polycyclic groups may be substituted or unsubstituted. Representative polycyclic groups may be substituted one or more times.

Halogen groups include F, Cl, Br, and I; nitro group refers to —NO₂; cyano group refers to —CN; isocyano group refers to —N≡C; epoxy groups encompass structures in which an oxygen atom is directly attached to two adjacent or non-adjacent carbon atoms of a carbon chain or ring system, which is essentially a cyclic ether structure. An epoxide is a cyclic ether with a three-atom ring.

An alkoxy group is a substituted or unsubstituted alkyl group, as defined above, singular bonded to oxygen. Alkoxy groups may be substituted or unsubstituted. Representative substituted alkoxy groups may be substituted one or more times. Exemplary alkoxy groups include, but are not limited to, methoxy, ethoxy, propoxy, butoxy, pentoxy, hexoxy, isopropoxy, sec-butoxy, tert-butoxy, cyclopropyloxy, cyclobutyloxy, cyclopentyloxy, and cyclohexyloxy groups.

As described herein, β-agent compounds of the present disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄R^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; SC(S)SR^{∘}; —(CH₂)₀₋₄SC(O)R^{∘}; —(CH₂)₀₋₄C(O)NR^{∘}₂; —C(S)NR^{∘}; —C(S)SR^{∘}; —SC(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘}may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘}together with their intervening atoms), are independently halogen, —(CH₂)₀₋₂R^{●}; -(haloR^{●}); —(CH₂)₀₋₂OH; —(CH₂)₀₋₂OR^{●}; —(CH₂)₀₋₂CH(OR^{●})₂; —O(haloR^{●}); —CN; —N₃; —(CH₂)₀₋₂C(O)R^{●}; —(CH₂)₀₋₂C(O)OH; —(CH₂)₀₋₂C(O)OR^{●}; —(CH₂)₀₋₂SR^{●}; —(CH₂)₀₋₂SH; —(CH₂)₀₋₂NH₂; —(CH₂)₀₋₂NHR^{●}; —(CH₂)₀₋₂NR^{●}₂; —NO₂, —SiR^{●}₃; —OSiR^{●}₃; —C(O)SR^{●}; —(C₁₋₄ straight or branched alkylene)C(O)OR^{●}; or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{●} include ═O and ═S.

Suitable divalent substituents on a saturated carbon atom of an “optionally substituted” group include the following: ═O; ═S; ═NNR*₂; ═NNHC(O)R*; ═NNHC(O)OR*; ═NNHS(O)₂R*; ═NR*; ═NOR*; —O(C(R*₂))₂₋₃O—; or —S(C(R*₂))₂₋₃S—; wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}; -(haloR^{●}); —OH, —OR^{●}; —O(haloR^{●}); —CN; —C(O)OH; —C(O)OR^{●}; —NH₂; —NHR^{●}; —NR^{●}₂, or —NO₂; wherein each R^{●} is unsubstituted or where preceded by “halo” is substituted only with one or more halogens, and is independently C₁₋₄ aliphatic, —CH₂Ph; —O(CH₂)₀₋₁Ph; or a 5-6-membered saturated; partially unsaturated; or aryl ring having 0-4 heteroatoms independently selected from nitrogen, oxygen, or sulfur.

Suitable substituents on a substitutable nitrogen of an “optionally substituted” group include —R^{†}; —NR^{†}₂; —C(O)R^{†}; —C(O)OR^{†}; —C(O)C(O)R^{†}; —C(O)CH₂C(O)R^{†}; —S(O)₂R^{†}; —S(O)₂NR^{†}₂; —C(S)NR^{†}₂; —C(NH)NR^{†}₂; or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}; -(haloR^{●}); —OH; —OR^{●}; —O(haloR^{●}); —CN; —C(O)OH; —C(O)OR^{●}; —NH₂; —NHR; —NR₂, or —NO₂; wherein each R^{●} is unsubstituted or where preceded by “halo” is substituted only with one or more halogens, and is independently C₁₋₄ aliphatic, —CH₂Ph; —O(CH₂)₀₋₁Ph; or a 5-6-membered saturated; partially unsaturated; or aryl ring having 0-4 heteroatoms independently selected from nitrogen, oxygen, or sulfur.

Thiol refers to —SH. Thiocarbonyl refers to (═S). Sulfonyl refers to —SO₂-alkyl, —SO₂-substituted alkyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclyl, and —SO₂-substituted heterocyclyl. Sulfonylamino refers to —NR^{a}SO₂alkyl, —NR^{a}SO₂-substituted alkyl, —NR^{a}SO₂cycloalkyl, —NR^{a}SO₂substituted cycloalkyl, —NR^{a}SO₂aryl, —NR^{a}SO₂substituted aryl, —NR^{a}SO₂heteroaryl, —NR^{a}SO₂ substituted heteroaryl, —NR^{a}SO₂heterocyclyl, —NR^{a}SO₂ substituted heterocyclyl, wherein each R^{a} independently is as defined herein.

Carboxyl refers to —COOH or salts thereof carboxyester refers to —C(O)O-alkyl, —C(O)O— substituted alkyl, —C(O)O-aryl, —C(O)O-substituted aryl, —C(O)β-cycloalkyl, —C(O)O-substituted cycloalkyl, —C(O)O-heteroaryl, —C(O)O-substituted heteroaryl, —C(O)O— heterocyclyl, and —C(O)O-substituted heterocyclyl. (Carboxyester)amino refers to —NR^{a}—C(O)O-alkyl, —NR^{a}—C(O)O-substituted alkyl, —NR^{a}—C(O)O-aryl, —NR^{a}—C(O)O-substituted aryl, —NR^{a}—C(O)β-cycloalkyl, —NR^{a}—C(O)O-substituted cycloalkyl, —NR^{a}—C(O)O— heteroaryl, —NR^{a}—C(O)O-substituted heteroaryl, —NR^{a}—C(O)O-heterocyclyl, and —NR^{a}—C(O)O-substituted heterocyclyl, wherein R^{a} is as recited herein. (Carboxyester)oxy refers to —O—C(O)O-alkyl, —O—C(O)O— substituted alkyl, —O—C(O)O-aryl, —O—C(O)O-substituted aryl, —O—C(O)β-cycloalkyl, —O—C(O)O-substituted cycloalkyl, —O—C(O)O-heteroaryl, —O—C(O)O-substituted heteroaryl, —O—C(O)O-heterocyclyl, and —O—C(O)O-substituted heterocyclyl. Oxo refers to (═O).

The terms “amine” and “amino” refer to derivatives of ammonia, wherein one of more hydrogen atoms have been replaced by a substituent which include, but are not limited to alkyl, alkenyl, aryl, and heterocyclyl groups. In some embodiments, substituted amino can include —NH—CO—R. Carbamate groups refers to —O(C═O)NR₁R₂, where R₁ and R₂ are independently hydrogen, aliphatic groups, aryl groups, or heterocyclyl groups.

Aminocarbonyl refers to —C(O)N(R^{b})₂, wherein each R^{b} independently is selected from hydrogen, alkyl, substituted alkyl, aryl, substituted aryl, cycloalkyl, substituted cycloalkyl, heteroaryl, substituted heteroaryl, heterocyclyl, substituted heterocyclyl. Also, each R^{b} may optionally be joined together with the nitrogen bound thereto to form a heterocyclyl or substituted heterocyclyl group, provided that both R^{b} are not both hydrogen. Aminocarbonylalkyl refers to -alkylC(O)N(R^{b})₂, wherein each R^{b} independently is selected from hydrogen, alkyl, substituted alkyl, aryl, substituted aryl, cycloalkyl, substituted cycloalkyl, heteroaryl, substituted heteroaryl, heterocyclyl, substituted heterocyclyl. Also, each R^{b} may optionally be joined together with the nitrogen bound thereto to form a heterocyclyl or substituted heterocyclyl group, provided that both R^{b} are not both hydrogen. Aminocarbonylamino refers to —NR^{a}C(O)N(R^{b})₂, wherein R^{a} and each R^{b} are as defined herein. Aminodicarbonylamino refers to —NR^{a}C(O)C(O)N(R^{b})₂, wherein R^{a} and each R^{b} are as defined herein. Aminocarbonyloxy refers to —O—C(O)N(R^{b})₂, wherein each R^{b} independently is as defined herein. Aminosulfonyl refers to —SO₂N(R^{b})₂, wherein each R^{b} independently is as defined herein.

Imino refers to —N═R^{c} wherein R^{c} may be selected from hydrogen, aminocarbonylalkyloxy, substituted aminocarbonylalkyloxy, aminocarbonylalkylamino, and substituted aminocarbonylalkylamino.

Additionally, unless otherwise stated, structures depicted herein are also meant to include compounds that differ only in the presence of one or more isotopically enriched atoms. For example, compounds having the present structures including the replacement of hydrogen by deuterium (e.g., D or H²) or tritium (e.g., T or H³), or the replacement of a carbon by a ¹³C- or ¹⁴C-enriched carbon are included and are within the scope of this invention. Such compounds are useful, for example, as analytical tools, as probes in biological assays, or as therapeutic agents in accordance with the present invention

Pharmaceutically acceptable salts of compounds described herein include conventional nontoxic salts or quaternary ammonium salts of a compound, e.g., from nontoxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. In other cases, described compounds may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

“Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)).

Disclosed herein is a β-agent compound according to Formula (I) or an optically pure stereoisomer, pharmaceutically acceptable salt, solvate, or prodrug thereof

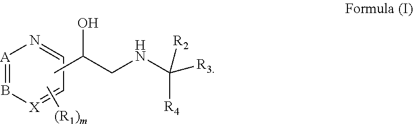

Each A, B, and X can be independently a nitrogen or carbon. Each R₁ can be independently hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, or unsubstituted or substituted heteroaryl. m can be an integer selected from 0 to 4.

R₂, R₃, and R₄ can be independently H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

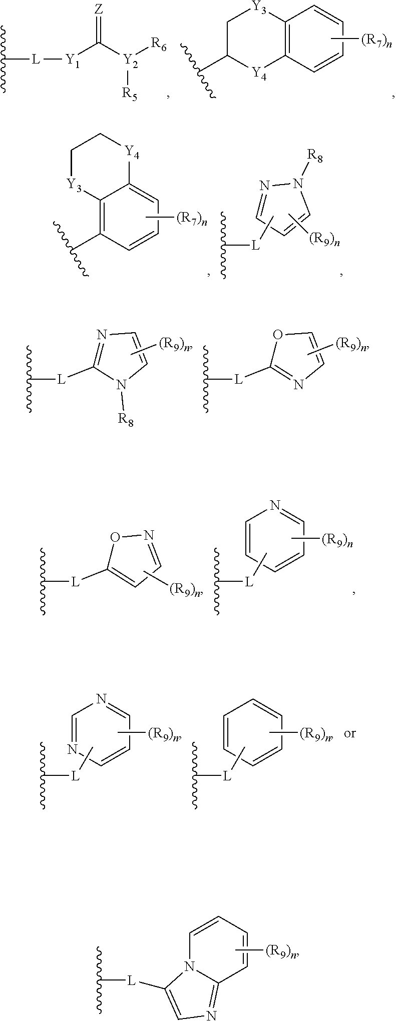

or R₂ and R₃ together with the carbon can form an unsubstituted or substituted 3-7 membered cycloalkyl or heterocycle ring.

L can be a C1-C5 alkyl linker optionally substituted, each Y₁, Y₂, Y₃, and Y₄ can be independently a covalent bond, a carbon, an oxygen, or a nitrogen, optionally substituted with hydrogen, unsubstituted or substituted alkyl, or unsubstituted or substituted cycloalkyl, and Z can be O or S.

R₅ and R₆ can be independently hydrogen, unsubstituted or substituted alkyl, or R₅ and R₆ are cyclically linked and together with Y₂ to form an optionally substituted cycloalkyl or heterocycle, each R₇ is independently selected from the group consisting of hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, or unsubstituted or substituted heteroaryl.

n can be an integer selected from 0 to 4, R₈ can be hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, or unsubstituted or substituted amino.

Also disclosed herein is a β-agent compound according to Formula (II) or an optically pure stereoisomer, pharmaceutically acceptable salt, solvate, or prodrug thereof

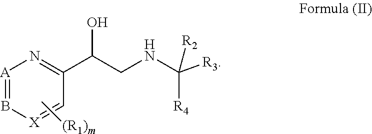

Each A, B, and X can be independently a nitrogen or carbon. Each R₁ can be hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, or unsubstituted or substituted heteroaryl. m can be an integer selected from 0 to 4.

R₂, R₃, and R₄ can be independently H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

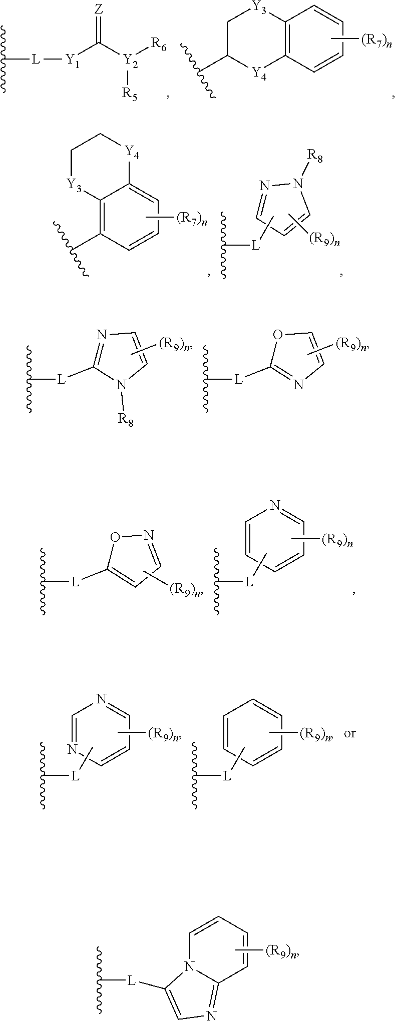

or R₂ and R₃ together with the carbon can form an unsubstituted or substituted 3-7 membered cycloalkyl or heterocycle ring.

L can be a C1-C5 alkyl linker optionally substituted, each Y₁, Y₂, Y₃, and Y₄ can be independently a covalent bond, a carbon, an oxygen, or a nitrogen, optionally substituted with hydrogen, unsubstituted or substituted alkyl, or unsubstituted or substituted cycloalkyl, and Z can be O or S.

R₅ and R₆ can be independently hydrogen, unsubstituted or substituted alkyl, or R₅ and R₆ can be cyclically linked and together with Y₂ to form an optionally substituted cycloalkyl or heterocycle, each R₇ can be hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, or unsubstituted or substituted heteroaryl.

n can be an integer selected from 0 to 4, R₈ can be hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, or unsubstituted or substituted amino.

Further disclosed herein is a compound according to Formula (III) or an optically pure stereoisomer, pharmaceutically acceptable salt, solvate, or prodrug thereof

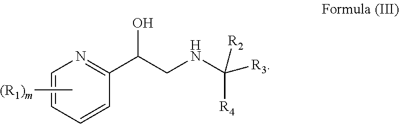

Each R₁ can be independently hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, or unsubstituted or substituted heteroaryl. m can be an integer selected from 0 to 4.

R₂, R₃, and R₄ can be independently H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

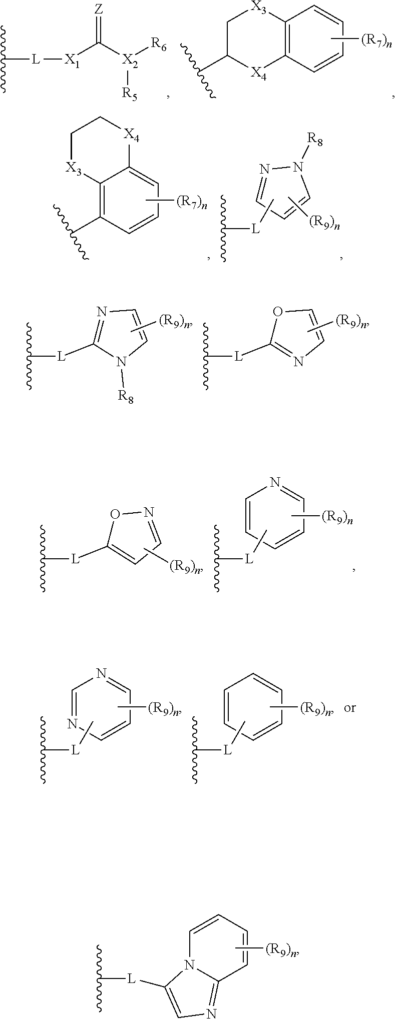

or R₂ and R₃ together with the carbon can form an unsubstituted or substituted 3-7 membered cycloalkyl or heterocycle ring.

L can be a C1-C5 alkyl linker optionally substituted, each X₁, X₂, X₃, and X₄ can be independently a covalent bond, a carbon, an oxygen, or a nitrogen, optionally substituted with hydrogen, unsubstituted or substituted alkyl, or unsubstituted or substituted cycloalkyl, and Y can be O or S.

R₅ and R₆ can be independently hydrogen, unsubstituted or substituted alkyl, or R₅ and R₆ can be cyclically linked and together with Y₂ to form an optionally substituted cycloalkyl or heterocycle, each R₇ can be independently hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, or unsubstituted or substituted heteroaryl.

n can be an integer selected from 0 to 4, R₈ can be hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, or unsubstituted or substituted amino.

Further disclosed herein is a β-agent compound according to Formula (I′):

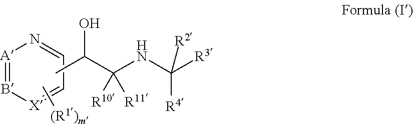

or a pharmaceutically acceptable salt thereof,
   wherein:
   A′, B′, and X′ are each independently nitrogen or carbon;
   each R^{1′} is independently halogen, —R′, —CN, —NO₂, —SF₅, —OR^{x}, —NR^{x}₂, —NHR^{x}, —SO₂R′, —C(O)R′, —C(O)NR′₂;
   each R′ is independently hydrogen or an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
   each R^{x} is independently an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
   m′ is an integer selected from 0 to 4;
   R^{2′}, R^{3′}, and R^{4′} are each independently halogen, —R′, —CN, —NO₂, —OR′, —NR′₂,

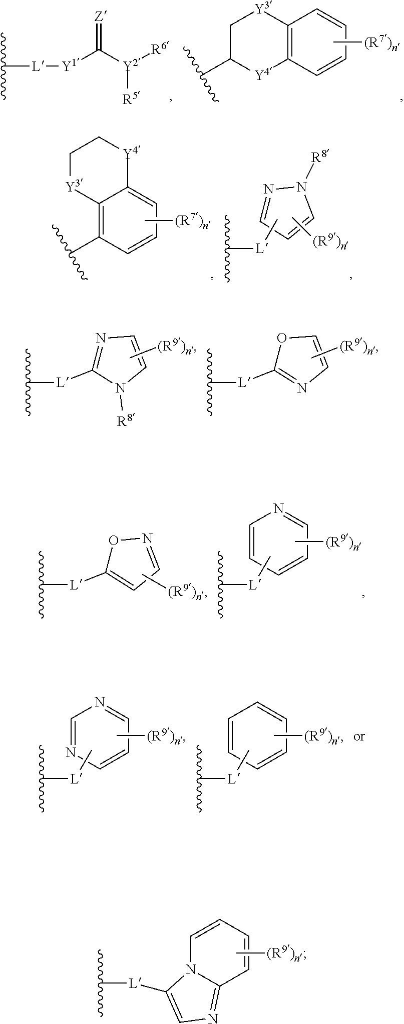

or
R²′ and R³′ together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;
L′ is optionally substituted C₁₋₅ alkylene;
Y^{1′}, Y^{2′}, Y^{3′}, and Y^{4′} are each independently a covalent bond, a carbon, an oxygen, or a nitrogen, optionally substituted with hydrogen, an optionally substituted C₁₋₆ alkyl, or an optionally substituted 3-7 membered saturated carbocyclic ring;
Z′ is O or S;
R^{5′} and R^{6′} are each independently hydrogen or optionally substituted alkyl, or
R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur;
each R^{7′} is independently —R′, halogen, —CN, —NO₂, —NR′₂, or —OR′;
n′ is an integer selected from 0 to 4;
R^{8′} is hydrogen, —CN, optionally substituted alkyl, or an optionally substituted aryl ring;
each R^{9′} is independently hydrogen, halogen, —CN, —OR^{x}, —NR′₂, or optionally substituted alkyl; and
R^{10′} and R^{11′} are each independently hydrogen or optionally substituted C₁₋₂ aliphatic.

Further disclosed herein is a β-agent compound according to Formula (I″):

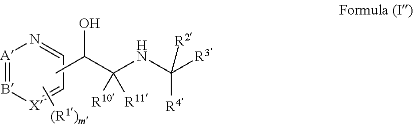

or a pharmaceutically acceptable salt thereof,
wherein:
   A′, B′, and X′ are each independently nitrogen or carbon;
   each R^{1′} is independently halogen, —R′, —CN, —NO₂, —SF₅, —OR^{x}, —NR^{x}₂, —NHR^{x}, —SO₂R′, —C(O)R′, —C(O)NR′₂, —NR′C(O)R′, —NR′CO₂R′, or —CO₂R′;
   each R′ is independently hydrogen or an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
   each R^{x} is independently an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
   m′ is an integer selected from 0 to 4;
   R^{2′}, R^{3′}, and R^{4′} are each independently halogen, —R′, —CN, —NO₂, —OR′, —NR′₂,

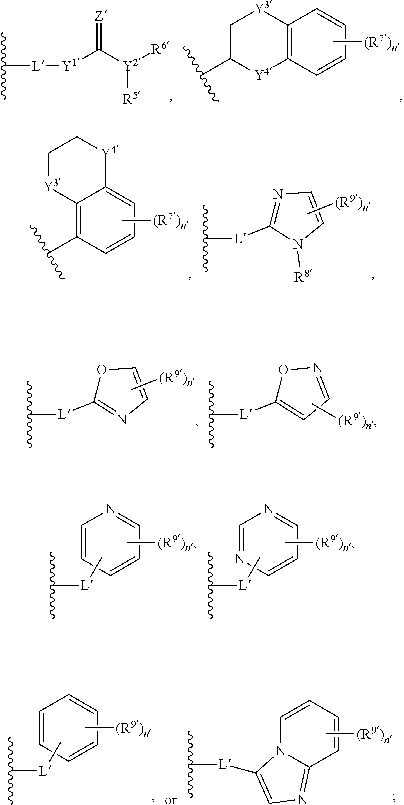

or
R²′ and R³′ together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;
L′ is optionally substituted C₁₋₅ alkylene;
Y^{1′}, Y^{2′}, Y^{3′}, and Y^{4′} are each independently a covalent bond, a carbon, an oxygen, or a nitrogen, optionally substituted with hydrogen, an optionally substituted C₁₋₆ alkyl, or an optionally substituted 3-7 membered saturated carbocyclic ring;
Z′ is O or S;
R^{5′} and R^{6′} are each independently hydrogen or optionally substituted alkyl, or
R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur;
each R^{7′} is independently —R′, halogen, —CN, —NO₂, —NR′₂, or —OR′;
n′ is an integer selected from 0 to 4;
R^{8′} is hydrogen, —CN, optionally substituted alkyl, or an optionally substituted aryl ring;
each R^{9′} is independently hydrogen, halogen, —CN, —OR^{x}, —NR′₂, or optionally substituted alkyl; and
R^{10′} and R^{11′} are each independently hydrogen or optionally substituted C₁₋₂ aliphatic.

As defined above and described herein, A′ is nitrogen or carbon. In some embodiments A′ is nitrogen. In some embodiments A′ is carbon.

In some embodiments A′ is selected from those depicted in Table 1, below.

As defined above and described herein, B′ is nitrogen or carbon. In some embodiments B′ is nitrogen. In some embodiments B′ is carbon.

In some embodiments B′ is selected from those depicted in Table 1, below.

As defined above and described herein, X′ is nitrogen or carbon. In some embodiments X′ is nitrogen. In some embodiments X′ is carbon.

In some embodiments X′ is selected from those depicted in Table 1, below.

As defined above, each R^{1′} is independently halogen, —R′, —CN, —NO₂, —SF₅, —OR^{x}, —NR^{x}₂, —NHR^{x}, —SO₂R′, —C(O)R′, —C(O)NR′₂, —NR′C(O)R′, —NR′CO₂R′, or —CO₂R′.

In some embodiments, R^{1′} is hydrogen. In some embodiments, R^{1′} is halogen. In some embodiments, R^{1′} is —R′. In some embodiments, R^{1′} is cyano. In some embodiments, R^{1′} is —NO₂. In some embodiments, R^{1′} is —SF₅. In some embodiments, R^{1′} is —OR^{x}. In some embodiments, R^{1′} is —NR^{x}₂. In some embodiments, R^{1′} is —NHR^{x}. In some embodiments, R^{1′} is —SO₂R′. In some embodiments, R^{1′} is —C(O)R′. In some embodiments, R^{1′} is —C(O)NR′₂. In some embodiments, R^{1′} is —NR′C(O)R′. In some embodiments, R^{1′} is —NR′CO₂R′. In some embodiments, R^{1′} is —CO₂R′.

In some embodiments, R^{1′} is —Br. In some embodiments, R^{1′} is —Cl. In some embodiments, R^{1′} is —F.

In some embodiments, R^{1′} is —CH₃. In some embodiments, R^{1′} is —CH₂CH₃. In some embodiments, R^{1′} is —CH(CH₃)₂.

In some embodiments, R^{1′} is —CF₃. In some embodiments, R^{1′} is —CF₂H. In some embodiments, R^{1′} is —CFH₂. In some embodiments, R^{1′} is —CF₂CH₃. In some embodiments, R^{1′} is —CH₂CF₃. In some embodiments, R^{1′} is —C≡CCH. In some embodiments, R^{1′} is vinyl. In some embodiments, R^{1′} is —C≡CCF₃. In some embodiments, R^{1′} is —CO₂H.

In some embodiments, R^{1′} is —CN.

In some embodiments, R^{1′} is —OCH₃. In some embodiments, R^{1′} is —OCH₂CH₃. In some embodiments, R^{1′} is —OCH(CH₃)₂. In some embodiments, R^{1′} is —OCF₃. In some embodiments, R^{1′} is —NHCH₃. In some embodiments, R^{1′} is —NHCD₃. In some embodiments, R^{1′} is —N(CD₃)CO₂tBu. In some embodiments, R^{1′} is —NHCH₂CH₃. In some embodiments, R^{1′} is —NHCH₂(CH₃)₂. In some embodiments, R^{1′} is —NHCH₂CF₃. In some embodiments, R^{1′} is —NHPh. In some embodiments, R^{1′} is —NHAc. In some embodiments, R^{1′} is —N(CH₃)₂. In some embodiments, R^{1′} is

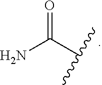 [SMILES: *C(N)=O]

In some embodiments, R^{1′} is

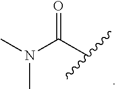 [SMILES: *C(=O)N(C)C]

In some embodiments, R^{1′} is

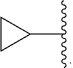 [SMILES: *C1CC1]

In some embodiments, R^{1′} is

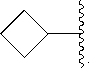 [SMILES: *C1CCC1]

In some embodiments, R^{1′} is

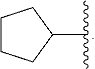 [SMILES: *C1CCCC1]

In some embodiments, R^{1′} is

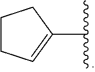 [SMILES: *C1=CCCC1]

In some embodiments, R^{1′} is

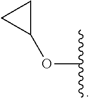 [SMILES: *OC1CC1]

In some embodiments, R^{1′} is

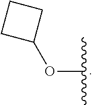 [SMILES: *OC1CCC1]

In some embodiments, R^{1′} is

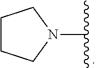 [SMILES: *N1CCCC1]

In some embodiments, R^{1′} is

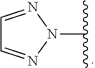 [SMILES: *n1nccn1]

In some embodiments, R^{1′} is

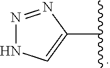 [SMILES: *c1c[nH]nn1]

In some embodiments, R^{1′} is

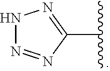 [SMILES: *c1nn[nH]n1]

In some embodiments, R^{1′} is

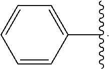 [SMILES: *c1ccccc1]

In some embodiments, R^{1′} is

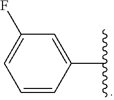 [SMILES: *c1cccc(F)c1]

In some embodiments, R^{1′} is

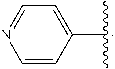 [SMILES: *c1ccncc1]

In some embodiments, R^{1′} is

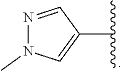 [SMILES: *c1cnn(C)c1]

In some embodiments, R^{1′} is

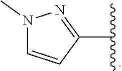 [SMILES: *c1ccn(C)n1]

In some embodiments, R^{1′} is

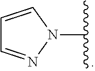 [SMILES: *n1cccn1]

In some embodiments, R^{1′} is

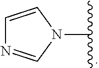 [SMILES: *n1ccnc1]

In some embodiments, R^{1′} is

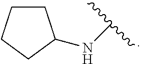 [SMILES: *NC1CCCC1]

In some embodiments, R^{1′} is

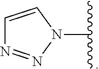 [SMILES: *n1ccnn1 ; "."]

In some embodiments, R^{1′} is

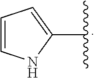 [SMILES: *c1ccc[nH]1]

In some embodiments, R^{1′} is

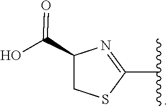 [SMILES: *C1=N[C@H](C(=O)O)CS1]

In some embodiments, R^{1′} is

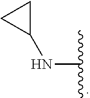 [SMILES: *NC1CC1]

In some embodiments, R^{1′} is

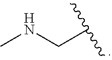 [SMILES: *CNC]

In some embodiments, R^{1′} is

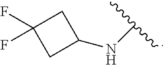 [SMILES: *NC1CC(F)(F)C1]

In some embodiments, R^{1′} is

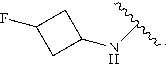 [SMILES: *NC1CC(F)C1]

In some embodiments, R^{1′} is

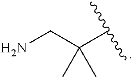 [SMILES: *C(C)(C)CN]

In some embodiments, R^{1′} is selected from those depicted in Table 1, below.

As defined above, each R′ is independently hydrogen or an optionally substituted group selected from C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur.

In some embodiments, R′ is hydrogen.

In some embodiments, R′ is an optionally substituted C₁₋₆ aliphatic. For instance, in some embodiments, R′ is —CF₃, —CF₂H, or —CFH₂.

In some embodiments, R′ is an optionally substituted 3-8 membered saturated monocyclic carbocyclic ring.

In some embodiments, R′ is an optionally substituted 3-8 membered partially unsaturated monocyclic carbocyclic ring.

In some embodiments, R′ is an optionally substituted phenyl.

In some embodiments, R′ is an optionally substituted 8-10 membered bicyclic partially unsaturated carbocyclic ring.

In some embodiments, R′ is an optionally substituted 8-10 membered bicyclic aromatic carbocyclic ring.

In some embodiments, R′ is an optionally substituted 4-8 membered saturated monocyclic heterocyclic ring having 1-2 heteroatoms independently selected from nitrogen, oxygen, or sulfur.

In some embodiments, R′ is an optionally substituted 4-8 membered partially unsaturated monocyclic heterocyclic ring having 1-2 heteroatoms independently selected from nitrogen, oxygen, or sulfur.

In some embodiments, R′ is an optionally substituted 5-6 membered monocyclic heteroaromatic ring having 1-4 heteroatoms independently selected from nitrogen, oxygen, or sulfur.

In some embodiments, R′ is an optionally substituted 8-10 membered bicyclic partially unsaturated ring having 1-5 heteroatoms independently selected from nitrogen, oxygen, or sulfur.

In some embodiments, R′ is an optionally substituted 8-10 membered bicyclic heteroaromatic ring having 1-5 heteroatoms independently selected from nitrogen, oxygen, or sulfur.

In some embodiments, R′ is selected from those depicted in Table 1, below.

As defined above, each R^{x} is independently an optionally substituted group selected from C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur.

In some embodiments, R^{x} is an optionally substituted C₁₋₆ aliphatic. For instance, in some embodiments, R^{x} is —CF₃, —CF₂H, or —CFH₂. In some embodiments, R^{x} is C₁₋₆ alkyl.

As defined above, m′ is an integer selected from 0 to 4.

In some embodiments, m′ is 0. In some embodiments, m′ is 1. In some embodiments, m′ is 2. In some embodiments, m′ is 3. In some embodiments, m′ is 4.

As defined above, R^{2′}, R^{3′}, and R^{4′} are each independently halogen, —R′, —CN, —OH, —OR′, —NR′₂, —NHR′, —NH₂,

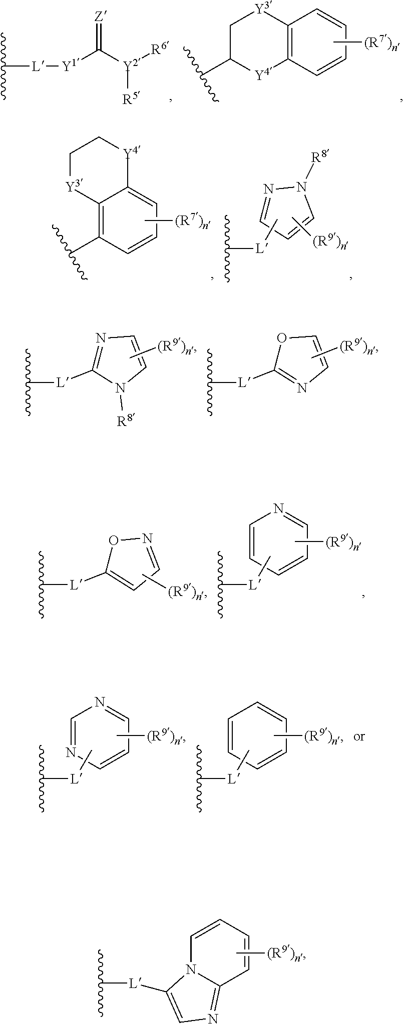

or R^{2′} and R^{3′} together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;

In some embodiments, R^{2′} is hydrogen. In some embodiments, R^{2′} is halogen. In some embodiments, R^{2′} is —R′. In some embodiments, R^{2′} is —CN. In some embodiments, R^{2′} is —NO₂. In some embodiments, R^{2′} is —OH. In some embodiments, R^{2′} is —OR′. In some embodiments, R^{2′} is —NR′₂. In some embodiments, R^{2′} is —NHR′. In some embodiments, R^{2′} is —NH₂.

In some embodiments, R^{2′} is

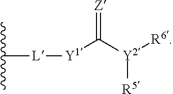

In some embodiments, R^{2′} is

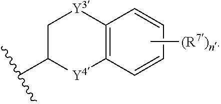

In some embodiments, R^{2′} is

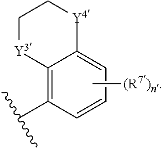

In some embodiments, R^{2′} is

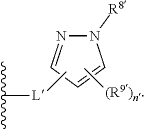

In some embodiments, R^{2′} is

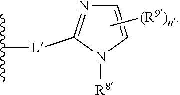

In some embodiments, R^{2′} is

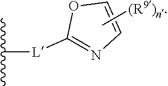

In some embodiments, R^{2′} is

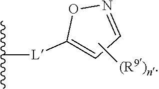

In some embodiments, R^{2′} is

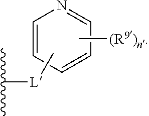

In some embodiments, R^{2′} is

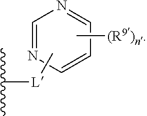

In some embodiments, R² is

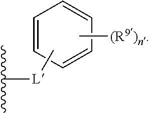

In some embodiments, R^{2′} is

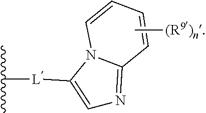

In some embodiments, R^{2′} is hydrogen. In some embodiments, R^{2′} is deuterium. In some embodiments, R² is —CH₃. In some embodiments, R^{2′} is —CD₃. In some embodiments, R^{2′} is

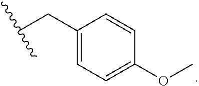 [SMILES: *Cc1ccc(OC)cc1]

In some embodiments, R^{3′} is hydrogen. In some embodiments, R^{3′} is halogen. In some embodiments, R^{3′} is —R′. In some embodiments, R^{3′} is —CN. In some embodiments, R^{3′} is —NO₂. In some embodiments, R^{3′} is —OH. In some embodiments, R^{3′} is —OR′. In some embodiments, R^{3′} is —NR′₂. In some embodiments, R^{3′} is —NHR′. In some embodiments, R^{3′} is —NH₂.

In some embodiments, R^{3′} is

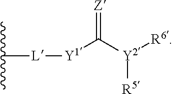

In some embodiments, R^{3′} is

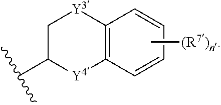

In some embodiments, R^{3′} is

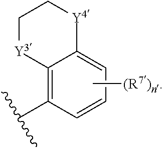

In some embodiments, R^{3′} is

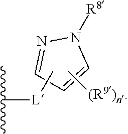

In some embodiments, R^{3′} is

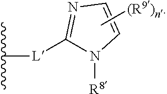

In some embodiments, R^{3′} is

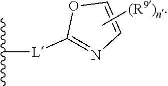

In some embodiments, R^{3′} is

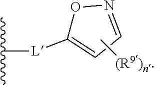

In some embodiments, R^{3′} is

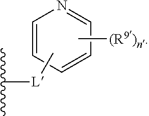

In some embodiments, R^{3′} is

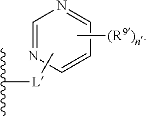

In some embodiments, R^{3′} is

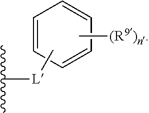

In some embodiments, R^{3′} is

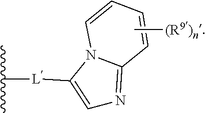

In some embodiments, R^{3′} is hydrogen. In some embodiments, R^{3′} is deuterium. In some embodiments, R^{3′} is —CH₃. In some embodiments, R^{3′} is —CD₃. In some embodiments, R^{3′} is

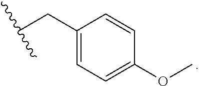 [SMILES: *Cc1ccc(OC)cc1]

In some embodiments, R^{2′} and R^{3′} together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur.

In some embodiments, R^{2′} and R^{3′} together with the carbon form an optionally substituted 3-7 membered saturated carbocyclic ring.

In some embodiments, R^{2′} and R^{3′} together with the carbon form an optionally substituted 5-6 membered monocyclic heteroaryl ring having 1-4 heteroatoms independently selected from nitrogen, oxygen, and sulfur.

In some embodiments, R^{2′} and R^{3′} together with the carbon form an optionally substituted 3-7 membered saturated or a partially unsaturated monocyclic heterocyclic ring having 1-2 heteroatoms independently selected from nitrogen, oxygen, and sulfur.

In some embodiments, R^{2′} and R^{3′} together with the carbon form

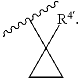

In some embodiments, R^{2′} and R^{3′} together with the carbon form

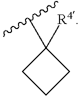

In some embodiments, R^{2′} and R^{3′} together with the carbon form

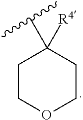

In some embodiments, R^{2′} and R^{3′} together with the carbon form

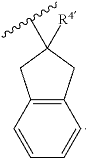

In some embodiments, R^{4′} is hydrogen. In some embodiments, R^{4′} is halogen. In some embodiments, R^{4′} is —R′. In some embodiments, R^{4′} is —CN. In some embodiments, R^{4′} is —NO₂. In some embodiments, R^{4′} is —OH. In some embodiments, R^{4′} is —OR′. In some embodiments, R^{4′} is —NR′₂. In some embodiments, R^{4′} is —NHR′. In some embodiments, R^{4′} is —NH₂. In some embodiments, R^{4′} is —CF₃.

In some embodiments, R^{4′} is

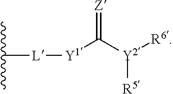

In some embodiments, R^{4′} is

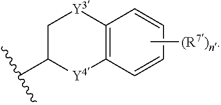

In some embodiments, R^{4′} is

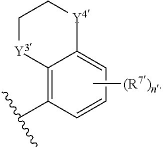

In some embodiments, R^{4′} is

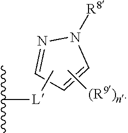

In some embodiments, R⁴ is

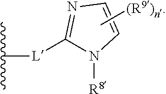

In some embodiments, R^{4′} is

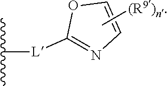

In some embodiments, R^{4′} is

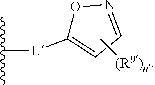

In some embodiments, R^{4′} is

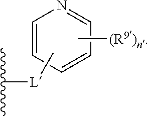

In some embodiments, R^{4′} is

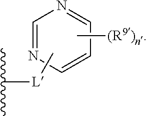

In some embodiments, R^{4′} is

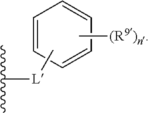

In some embodiments, R^{4′} is

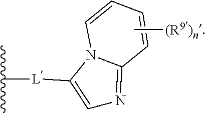

In some embodiments, R^{4′} is hydrogen. In some embodiments, R^{4′} is deuterium. In some embodiments, R^{4′} is —CH₃. In some embodiments, R^{4′} is —CD₃. some embodiments, R^{4′} is

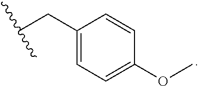 [SMILES: *Cc1ccc(OC)cc1]

In some embodiments, R^{2′}, R^{3′}, and R^{4′}, are each selected from those depicted in Table 1, below.

As defined above, L′ is optionally substituted C₁₋₅ alkylene.

In some embodiments, L′ is —CH₂—.

In some embodiments, L′ is selected from those depicted in Table 1, below.

As defined above, Y^{1′}, Y^{2′}, Y^{3′}, and Y^{4′} are each independently a covalent bond, a carbon, an oxygen; or a nitrogen, optionally substituted with hydrogen, an optionally substituted C₁₋₆ alkyl, or an optionally substituted 3-7 membered saturated carbocyclic ring.

In some embodiments, Y^{1′} is a covalent bond. In some embodiments, Y^{1′} is a carbon. In some embodiments, Y^{1′} is an oxygen. In some embodiments, Y^{1′} is a nitrogen, optionally substituted with hydrogen, an optionally substituted C₁₋₆ alkyl, or an optionally substituted 3-7 membered saturated carbocyclic ring.

In some embodiments, Y^{2′} is a covalent bond. In some embodiments, Y^{2′} is a carbon. In some embodiments, Y^{2′} is an oxygen. In some embodiments, Y^{2′} is a nitrogen, optionally substituted with hydrogen, an optionally substituted C₁₋₆ alkyl, or an optionally substituted 3-7 membered saturated carbocyclic ring.

In some embodiments, Y^{3′} is a covalent bond. In some embodiments, Y^{3′} is a carbon. In some embodiments, Y^{3′} is an oxygen. In some embodiments, Y^{3′} is a nitrogen, optionally substituted with hydrogen, an optionally substituted C₁₋₆ alkyl, or an optionally substituted 3-7 membered saturated carbocyclic ring.

In some embodiments, Y^{3′} is a covalent bond. In some embodiments, Y^{3′} is a carbon.

In some embodiments, Y^{4′} is a covalent bond. In some embodiments, Y^{4′} is a carbon. In some embodiments, Y^{4′} is an oxygen. In some embodiments, Y^{4′} is a nitrogen, optionally substituted with hydrogen, an optionally substituted C₁₋₆ alkyl, or an optionally substituted 3-7 membered saturated carbocyclic ring.

In some embodiments, Y^{4′} is a covalent bond. In some embodiments, Y^{4′} is a carbon.

In some embodiments, Y^{1′}, Y^{2′}, Y^{3′}, and Y^{4′} are each selected from those depicted in Table 1, below.

As defined above, Z′ is O or S.

In some embodiments, Z′ is O. In some embodiments, Z′ is S.

In some embodiments, Z′ is selected from those depicted in Table 1, below.

As defined above, R^{5′} and R^{6′} are each independently hydrogen or optionally substituted alkyl, or R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur.

In some embodiments, R^{5′} is hydrogen. In some embodiments, R^{5′} is an optionally substituted C₁₋₆ alkyl.

In some embodiments, R^{6′} is hydrogen. In some embodiments, R^{6′} is an optionally substituted C₁₋₆ alkyl.

In some embodiments, R^{5′} and R^{6′} are cyclically linked and together with Y^{2′} form an optionally substituted 3-7 membered saturated carbocyclic ring.

In some embodiments, R^{5′} and R^{6′} are cyclically linked and together with Y^{2′} form an optionally substituted 5-6 membered monocyclic heteroaryl ring having 1-4 heteroatoms independently selected from nitrogen, oxygen, and sulfur.

In some embodiments, R^{5′} and R^{6′} are cyclically linked and together with Y^{2′} form an optionally substituted 3-7 membered saturated or partially unsaturated monocyclic heterocyclic ring having 1-2 heteroatoms independently selected from nitrogen, oxygen, and sulfur.

In some embodiments, R^{5′} and R^{6′} are cyclically linked and together with Y^{2′} form an optionally substituted 7-12 membered saturated or partially unsaturated bicyclic heterocyclic ring having 1-4 heteroatoms independently selected from nitrogen, oxygen, and sulfur.

In some embodiments, R⁵′ and R⁶′ are each selected from those depicted in Table 1, below.

As defined above, each R⁷′ is independently —R′, halogen, —CN, —NO₂, —OH, —NR′₂, —NHR′, —NH₂, or —OR′.

In some embodiments, R^{7′} is hydrogen. In some embodiments, R^{7′} is halogen. In some embodiments, R^{7′} is —CN. In some embodiments, R^{7′} is —NO₂. In some embodiments, R^{7′} is —OH. In some embodiments, R^{7′} is —NR′₂. In some embodiments, R^{7′} is —NHR′. In some embodiments, R^{7′} is —NH₂. In some embodiments, R^{7′} is —OR′.

In some embodiments, each R^{7′} is independently selected from those depicted in Table 1, below.

As defined above, n′ is an integer selected from 0 to 4.

In some embodiments, n′ is 0. In some embodiments, n′ is 1. In some embodiments, n′ is 2. In some embodiments, n′ is 3. In some embodiments, n′ is 4.

As defined above, R^{8′} is hydrogen, —CN, optionally substituted alkyl, or an optionally substituted aryl ring.

In some embodiments, R^{8′} is hydrogen. In some embodiments, R^{1′} is —CN. In some embodiments, R^{8′} is an optionally substituted C₁₋₆ alkyl. In some embodiments, R^{1′} is an optionally substituted aryl ring.

In some embodiments, R^{8′} is selected from those depicted in Table 1, below.

As defined above, each R^{9′} is independently hydrogen, halogen, —CN, —OR^{x}, —NR′2, or optionally substituted alkyl.

In some embodiments, R^{9′} is hydrogen. In some embodiments, R^{9′} is halogen. In some embodiments, R^{9′} is —CN. In some embodiments, R^{9′} is —OR^{x}. In some embodiments, R^{9′} is —NR′₂. In some embodiments, R^{9′} is —NHR′. In some embodiments, R^{9′} is —NH₂. In some embodiments, R^{9′} is an optionally substituted C₁₋₆ alkyl.

In some embodiments, R^{9′} is selected from those depicted in Table 1, below.

As defined above, R^{10′} and R^{11′} are each independently hydrogen or optionally substituted C₁₋₂ aliphatic. In some embodiments, R^{10′} and R^{11′} are each independently hydrogen, methyl, or ethyl.

In some embodiments, R^{10′} is hydrogen. In some embodiment, R^{10′} is an optionally substituted C₁ aliphatic. In some embodiment, R^{10′} is methyl. In some embodiment, R^{10′} is an optionally substituted C₂ aliphatic. In some embodiment, R^{10′} is ethyl.

In some embodiments, R^{10′} is selected from those depicted in Table 1, below.

In some embodiments, R^{11′} is hydrogen. In some embodiment, R^{11′} is an optionally substituted C₁ aliphatic. In some embodiment, R^{11′} is methyl. In some embodiment, R^{11′} is an optionally substituted C₂ aliphatic. In some embodiment, R^{11′} is ethyl.

In some embodiments, R^{11′} is selected from those depicted in Table 1, below.

Further disclosed herein is a β-agent compound according to Formula (II′):

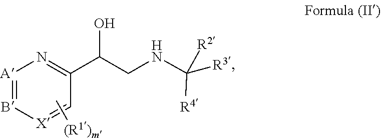

or a pharmaceutically acceptable salt thereof,
wherein each of A′, B′, X′, R^{1′}, R^{2′}, R^{3′}, R^{4′}, and m′ is as defined above and as described in embodiments provided herein, both singly and in combination.

Further disclosed herein is a β-agent compound according to Formula (III′):

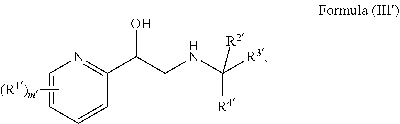

or a pharmaceutically acceptable salt thereof,
wherein each of R^{1′}, R^{2′}, R^{3′}, R^{4′}, and m′ is as defined above and as described in embodiments provided herein, both singly and in combination.

Further disclosed herein is a β-agent compound according to Formula (IV′):

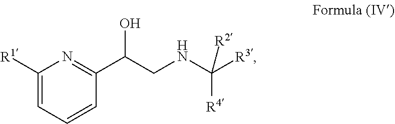

or a pharmaceutically acceptable salt thereof,
wherein each of R^{1′}, R^{2′}, R^{3′}, and R^{4′} is as defined above and as described in embodiments provided herein, both singly and in combination. In some such embodiments, R^{1′} is —CF₃. In some such embodiments, R^{1′} is —CF₂H. In some such embodiments, R^{1′} is —OCF₃. In some such embodiments, R^{1′} is —CN. In some such embodiments, R^{1′} is —C(O)NR′₂. In some such embodiments, R^{1′} is a cyclopropyl group. In some such embodiments, R^{1′} is a tetrazole. In some such embodiments, R^{1′} is phenyl. In some such embodiments, R^{1′} is —Br. In some such embodiments, R^{1′} is —CH₃.

Further disclosed herein is a β-agent compound according to Formula (V′):

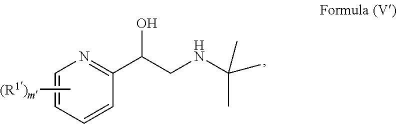

or a pharmaceutically acceptable salt thereof,
wherein each of R′ and m′ is as defined above and as described in embodiments provided herein, both singly and in combination.

Further disclosed herein is a β-agent compound according to Formula (VI′):

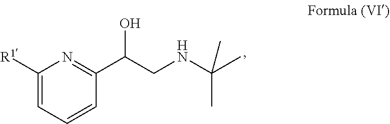

or a pharmaceutically acceptable salt thereof,
wherein R^{1′} is as defined above and as described in embodiments provided herein, both singly and in combination.

Further disclosed herein is a β-agent compound according to Formula (VII′):

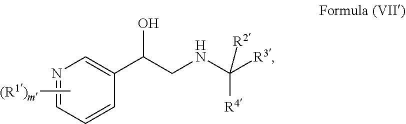

or a pharmaceutically acceptable salt thereof,
wherein each of R^{1′}, R^{2′}, R^{3′}, R^{4′}, and m′ is as defined above and as described in embodiments provided herein, both singly and in combination.

Further disclosed herein is a β-agent compound according to Formula (VIII′):

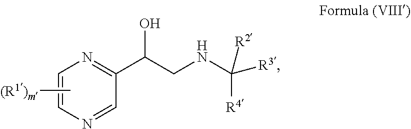

or a pharmaceutically acceptable salt thereof,
wherein each of R^{1′}, R^{2′}, R^{3′}, R^{4′}, and m′ is as defined above and as described in embodiments provided herein, both singly and in combination.

Further disclosed herein is a β-agent compound according to Formula (IX′):

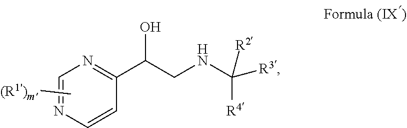

or a pharmaceutically acceptable salt thereof,
wherein each of R^{1′}, R^{2′}, R^{3′}, R^{4′}, and m′ is as defined above and as described in embodiments provided herein, both singly and in combination.

Further disclosed herein is a β-agent compound according to Formula (X′):

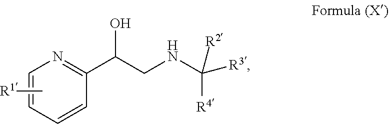

or a pharmaceutically acceptable salt thereof,
wherein
   R^{1′} is halogen, —R^{x}, —CN, —NO₂, —SF₅, —OR^{x}, —SO₂R′, or —C(O)R′;
   R^{2′}, R^{3′}, and R^{4′} are each independently halogen, —R′, —CN, —NO₂, —OR′, or —NR′2, or
   R^{2′} and R^{3′} together with the carbon form an optionally substituted 3-7 membered cycloalkyl or heterocycle ring; and
   R′ and R^{x} are as defined above and as described in embodiments provided herein, both singly and in combination. In some such embodiments, R^{1′} is —CF₃. In some such embodiments, R^{1′} is —CF₂H. In some such embodiments, R^{1′} is —OCF₃. In some such embodiments, R^{1′} is —CN. In some such embodiments, R^{1′} is —C(O)NR′₂. In some such embodiments, R^{1′} is a cyclopropyl group. In some such embodiments, R^{1′} is a tetrazole. In some such embodiments, R^{1′} is phenyl. In some such embodiments, R^{1′} is —Br. In some such embodiments, R^{1′} is —CH₃.

Further disclosed herein is a β-agent compound according to Formula (XI′):

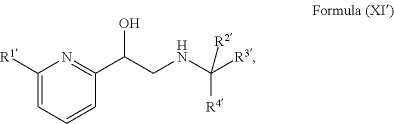

or a pharmaceutically acceptable salt thereof,
wherein:
   R^{1′} is halogen, —R′, —CN, —NO₂, —SF₅, —OR^{x}, —SO₂R′, or —C(O)R′;
   R^{2′}, R^{3′}, and R^{4′} are each independently halogen, —R′, —CN, —NO₂, —OR′, or —NR′₂, or
   R^{2′} and R^{3′} together with the carbon form an optionally substituted 3-7 membered cycloalkyl or heterocycle ring; and
   R′ and R^{x} are as defined above and as described in embodiments provided herein, both singly and in combination.

Further disclosed herein is a β-agent compound according to Formula (XII′):

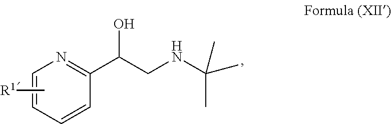

or a pharmaceutically acceptable salt thereof,
wherein:
   R^{1′} is halogen, —R′, —CN, —NO₂, —SF₅, —OR^{x}, —SO₂R′, or —C(O)R′; and
   R′ and R^{x} are as defined above and as described in embodiments provided herein, both singly and in combination.

Further disclosed herein is a compound according to Formula (XIII′):

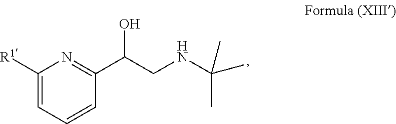

or a pharmaceutically acceptable salt thereof,
wherein:
   R^{1′} is halogen, —R′, —CN, —NO₂, —SF₅, —OR^{x}, —SO₂R′, or —C(O)R′; and
   R′ and R^{x} are as defined above and as described in embodiments provided herein, both singly and in combination.

Further disclosed herein is a β-agent compound according to Formula (XIV′):

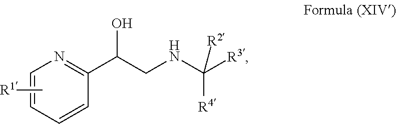

or a pharmaceutically acceptable salt thereof,
wherein
   R^{1′} is halogen, —R′, —CN, or —NO₂;
   R^{2′}, R^{3′}, and R^{4′} are each independently halogen, —R′, —CN, —NO₂, —OR′, or —NR′₂, or
   R²′ and R³′ together with the carbon form an optionally substituted 3-7 membered cycloalkyl or heterocycle ring; and
   R′ is as defined above and as described in embodiments provided herein, both singly and in combination.

Further disclosed herein is a compound according to Formula (XV′):

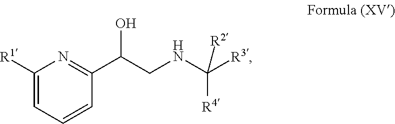

or a pharmaceutically acceptable salt thereof,
wherein:
   R^{1′} is halogen, —R′, —CN, or —NO₂;
   R^{2′}, R^{3′}, and R^{4′} are each independently halogen, —R′, —CN, —NO₂, —OR′, or —NR′₂, or
   R²′ and R³′ together with the carbon form an optionally substituted 3-7 membered cycloalkyl or heterocycle ring; and
   R′ is as defined above and as described in embodiments provided herein, both singly and in combination.

Further disclosed herein is a compound according to Formula (XVI′):

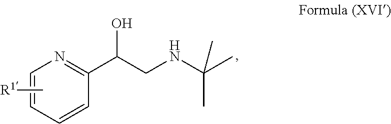

or a pharmaceutically acceptable salt thereof,
wherein:
   R^{1′} is halogen, —R′, —CN, or —NO₂; and
   R′ is as defined above and as described in embodiments provided herein, both singly and in combination.

Further disclosed herein is a compound according to Formula (XVII′):

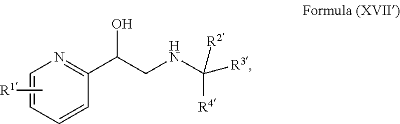

or a pharmaceutically acceptable salt thereof,
wherein
   R^{1′} is halogen, —R′, —CN, or —NO₂;
   each R′ is an optionally substituted C₁₋₆ aliphatic; and
   R^{2′}, R^{3′}, and R^{4′} are each independently halogen, —R′, —CN, —NO₂, —OR′, or —NR′₂, or
   R²′ and R³′ together with the carbon form an optionally substituted 3-7 membered cycloalkyl or heterocycle ring.

Further disclosed herein is a compound according to Formula (XVIII′):

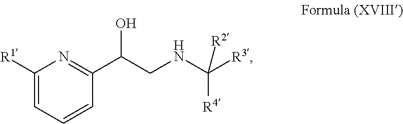

or a pharmaceutically acceptable salt thereof,
wherein:
   R^{1′} is halogen, —R′, —CN, or —NO₂;
   each R′ is an optionally substituted C₁₋₆ aliphatic; and
   R^{2′}, R^{3′}, and R^{4′} are each independently halogen, —R′, —CN, —NO₂, —OR′, or —NR′₂, or
   R²′ and R³′ together with the carbon form an optionally substituted 3-7 membered cycloalkyl or heterocycle ring.

Further disclosed herein is a compound according to Formula (XIX′):

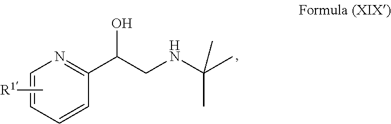

or a pharmaceutically acceptable salt thereof,
wherein:
   R^{1′} is halogen, —R′, —CN, or —NO₂; and
   R′ is optionally substituted C₁₋₆ aliphatic.

Further disclosed herein is a compound according to Formula (XX′):

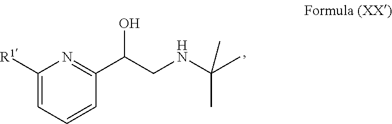

or a pharmaceutically acceptable salt thereof,
wherein:
   R^{1′} is halogen, —R′, —CN, or —NO₂; and
   R′ is an optionally substituted C₁₋₆ aliphatic.

Further disclosed herein is a compound according to Formula (XXI′):

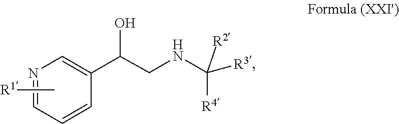

or a pharmaceutically acceptable salt thereof,
wherein
   R^{1′} is halogen, —R′, —CN, or —NO₂;
   each R′ is an optionally substituted C₁₋₆ aliphatic; and R^{2′}, R^{3′}, and R^{4′} are each independently halogen, —R′, —CN, —NO₂, —OR′, or —NR′₂, or
   R²′ and R³′ together with the carbon form an optionally substituted 3-7 membered cycloalkyl or heterocycle ring.

Further disclosed herein is a compound according to Formula (XXII′):

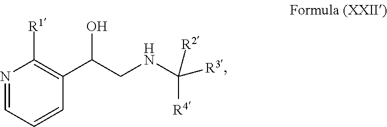

or a pharmaceutically acceptable salt thereof,
wherein:
   R^{1′} is halogen, —R′, —CN, or —NO₂;
   each R′ is an optionally substituted C₁₋₆ aliphatic; and
   R^{2′}, R^{3′}, and R^{4′} are each independently halogen, —R′, —CN, —NO₂, —OR′, or —NR′₂, or
   R²′ and R³′ together with the carbon form an optionally substituted 3-7 membered cycloalkyl or heterocycle ring.

Further disclosed herein is a compound according to Formula (XXIII′):

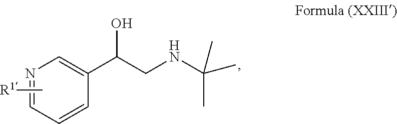

or a pharmaceutically acceptable salt thereof,
wherein:
   R^{1′} is halogen, —R′, —CN, or —NO₂; and
   R′ is optionally substituted C₁₋₆ aliphatic.

Further disclosed herein is a compound according to Formula (XXIV′):

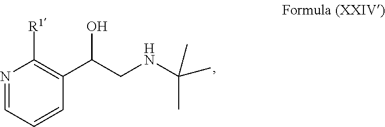

or a pharmaceutically acceptable salt thereof,
wherein:
   R^{1′} is halogen, —R′, —CN, or —NO₂; and
   R′ is an optionally substituted C₁₋₆ aliphatic.

Further disclosed herein is a compound according to Formula (XXV′):

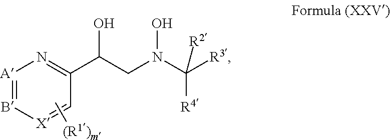

or a pharmaceutically acceptable salt thereof,
wherein each of A′, B′, X′, R^{1′}, R^{2′}, R^{3′}, R^{4′}, and m′ is as defined above and as described in embodiments provided herein, both singly and in combination.

TABLE 1
below illustrates exemplary ß-agent compounds of the disclosure.

| Compound No. | Chemical Structure | MS (m/z) (M + 1) |
|---|---|---|
| 03-1 | 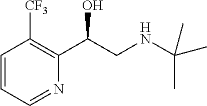 [SMILES: CC(C)(C)NC[C@H](O)c1ncccc1C(F)(F)F] | 263.2 |
| 03-2 | 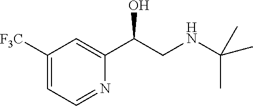 [SMILES: CC(C)(C)NC[C@H](O)c1cc(C(F)(F)F)ccn1] | 263.2 |
| 03-3 | 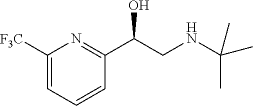 [SMILES: CC(C)(C)NC[C@H](O)c1cccc(C(F)(F)F)n1] | 263.2 |
| 03-4 | 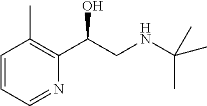 | 209.2 |
| 03-5 | 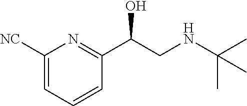 [SMILES: CC(C)(C)NC[C@H](O)c1cccc(C#N)n1] | 220.24 |
| 03-6 | 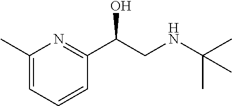 | 209.32 |
| 03-7 | 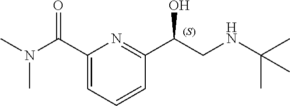 | 266.1 |
| 03-8 | 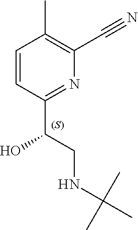 | 234.23 |
| 03-9 | 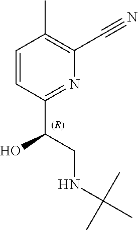 | 234.23 |
| 03-10 | 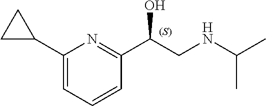 | 221.1 |
| 03-11 | 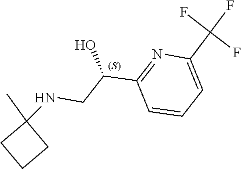 | 275.17 |
| 03-12 | 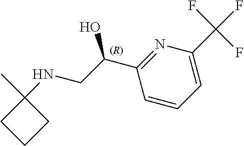 | 275.17 |
| 03-13 | 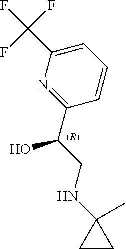 | 261.14 |
| 03-14 | 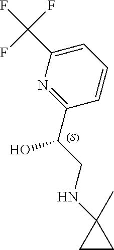 | 261.14 |
| 03-15 | 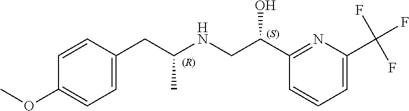 | 355.32 |
| 03-16 | 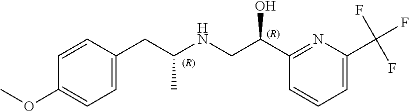 | 355.32 |
| 03-17 | 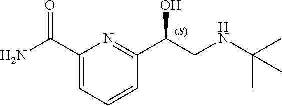 | 238.1 |
| 03-18 | 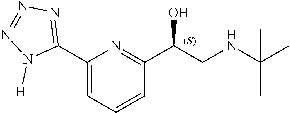 | 263.2 |
| 03-19 | 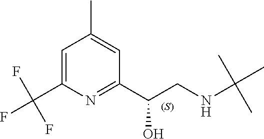 | 277.1 |
| 03-20 | 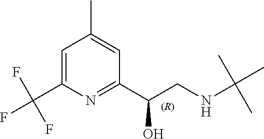 | 277.1 |
| 03-21 | 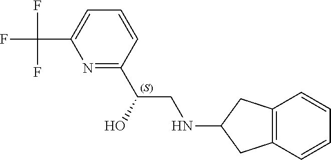 | 323.27 |
| 03-22 | 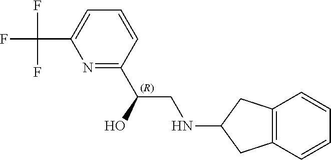 | 323.27 |
| 03-23 | 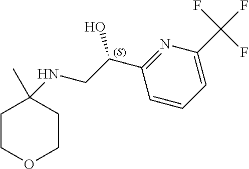 | 305.25 |
| 03-24 | 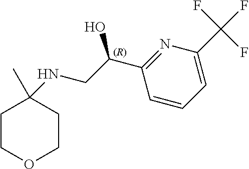 | 305.25 |
| 03-25 | 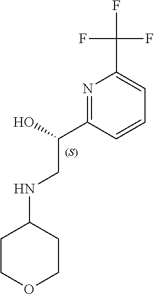 | 291.18 |
| 03-26 | 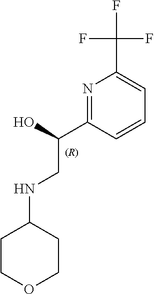 | 291.18 |
| 03-28 | 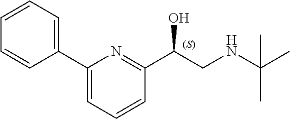 | 271.1 |
| 03-29 | 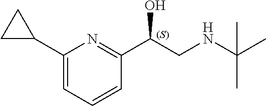 | 253.2 |
| 03-30 | 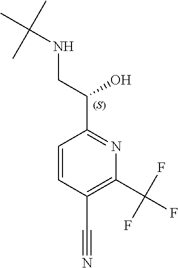 | 288.25 |
| 03-31 | 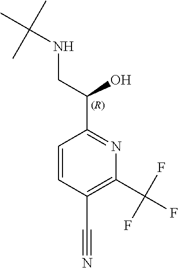 | 288.25 |
| 03-32 | 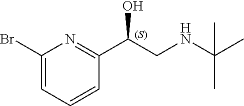 | 273.1 |
| 03-33 | 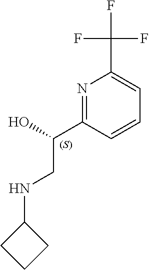 | 261.24 |
| 03-34 | 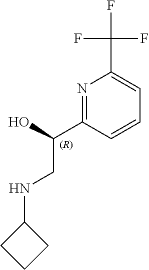 | 261.24 |
| 03-35 | 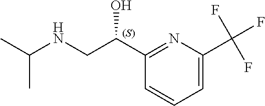 | 249.17 |
| 03-36 | 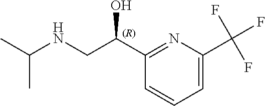 | 249.17 |
| 03-37 | 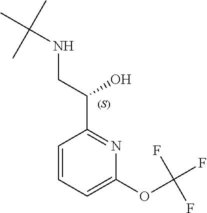 | 279.17 |
| 03-38 | 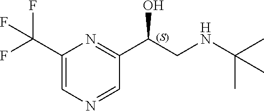 | 264.1 |
| 03-43 | 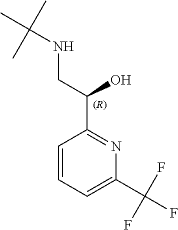 | 263.2 |
| 03-44 | 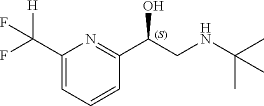 | 245.1 |
| 03-45 | 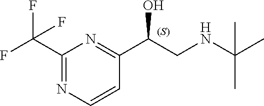 | 264.1 |
| 03-46 | 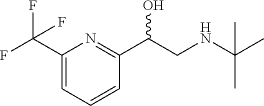 [SMILES: CC(C)(C)NCC(O)c1cccc(C(F)(F)F)n1] | 263.1 |
| 03-47 | 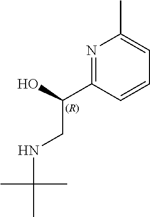 | 209.32 |
| 03-48 | 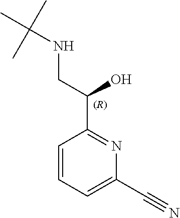 | 220.25 |
| 03-49 | 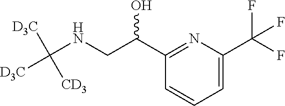 [SMILES: [2H]C([2H])([2H])C(NCC(O)c1cccc(C(F)(F)F)n1)(C([2H])([2H])[2H])C([2H])([2H])[2H]] | 272.1 |
| 03-50 | 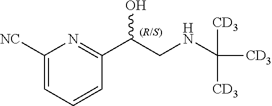 | 229.2 |
| 03-51 | 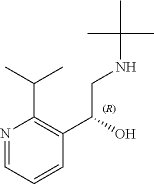 | 237.2 |
| 03-52 | 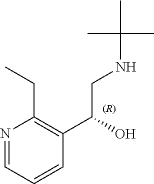 | 223.2 |
| 03-53 | 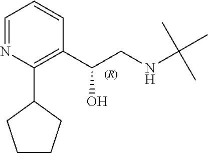 | 263.2 |
| 03-54 | 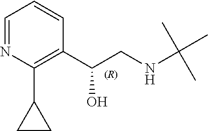 | 235.3 |
| 03-55 | 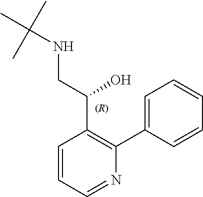 | 271.4 |
| 03-56 | 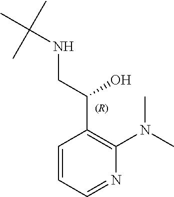 | 238.2 |
| 03-57 | 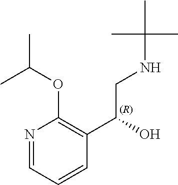 | 253.2 |
| 03-58 | 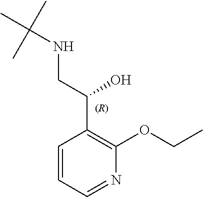 | 239.2 |
| 03-59 | 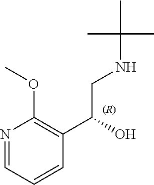 | 225.2 |
| 03-60 | 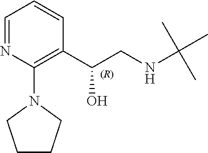 | 264.2 |
| 03-61 | 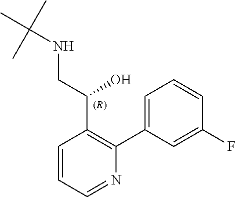 | 289.3 |
| 03-62 | 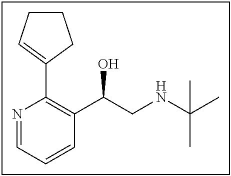 [SMILES: CC(C)(C)NC[C@H](O)c1cccnc1C1=CCCC1] | 261.4 |
| 03-63 | 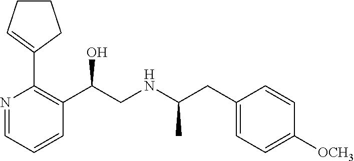 [SMILES: COc1ccc(C[C@@H](C)NC[C@H](O)c2cccnc2C2=CCCC2)cc1] | 353.5 |
| 03-64 | 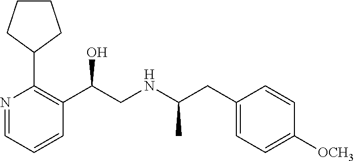 [SMILES: COc1ccc(C[C@@H](C)NC[C@H](O)c2cccnc2C2CCCC2)cc1] | 355.5 |
| 03-65 | 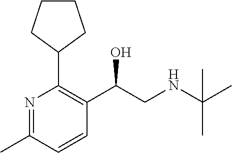 [SMILES: Cc1ccc([C@@H](O)CNC(C)(C)C)c(C2CCCC2)n1] | 277.4 |
| 03-66 | 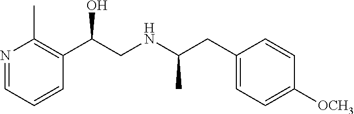 [SMILES: COc1ccc(C[C@@H](C)NC[C@H](O)c2cccnc2C)cc1] | 301.3 |
| 03-67 | 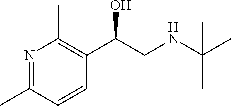 [SMILES: Cc1ccc([C@@H](O)CNC(C)(C)C)c(C)n1] | 223.3 |
| 03-68 | 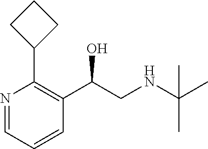 [SMILES: CC(C)(C)NC[C@H](O)c1cccnc1C1CCC1] | 249.3 |
| 03-70 | 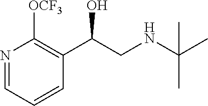 [SMILES: CC(C)(C)NC[C@H](O)c1cccnc1OC(F)(F)F] | 279.3 |
| 03-71 | 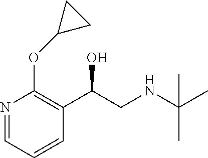 [SMILES: CC(C)(C)NC[C@H](O)c1cccnc1OC1CC1] | 251.3 |
| 03-72 | 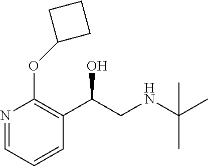 [SMILES: CC(C)(C)NC[C@H](O)c1cccnc1OC1CCC1] | 265.3 |
| 03-73 | 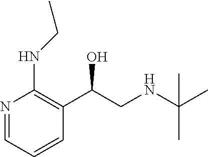 [SMILES: CCNc1ncccc1[C@@H](O)CNC(C)(C)C] | 238.4 |
| 03-74 | 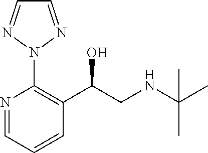 [SMILES: CC(C)(C)NC[C@H](O)c1cccnc1-n1nccn1] | 262.2 |
| 03-75 | 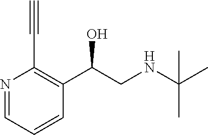 [SMILES: C#Cc1ncccc1[C@@H](O)CNC(C)(C)C] | 219.3 |
| 03-76 | 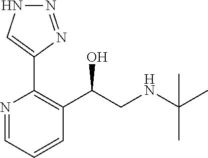 [SMILES: CC(C)(C)NC[C@H](O)c1cccnc1-c1c[nH]nn1] | 262.3 |
| 03-77 | 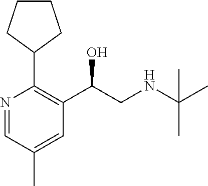 [SMILES: Cc1cnc(C2CCCC2)c([C@@H](O)CNC(C)(C)C)c1] | 277.4 |
| 03-78 | 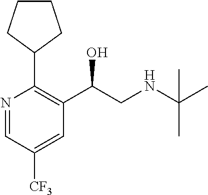 [SMILES: CC(C)(C)NC[C@H](O)c1cc(C(F)(F)F)cnc1C1CCCC1] | 331.3 |
| 03-79 | 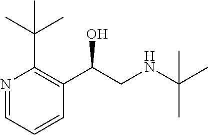 [SMILES: CC(C)(C)NC[C@H](O)c1cccnc1C(C)(C)C] | 251.4 |
| 03-80 | 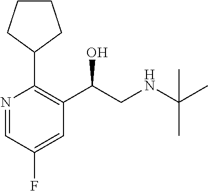 [SMILES: CC(C)(C)NC[C@H](O)c1cc(F)cnc1C1CCCC1] | 281.4 |
| 03-81 | 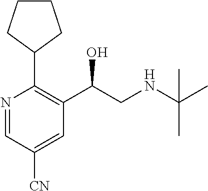 [SMILES: CC(C)(C)NC[C@H](O)c1cc(C#N)cnc1C1CCCC1] | |
| 03-82 | 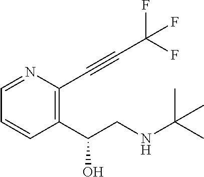 [SMILES: CC(C)(C)NC[C@H](O)c1cccnc1C#CC(F)(F)F] | |
| 03-83 | 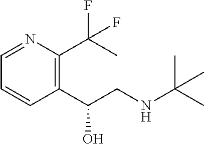 [SMILES: CC(C)(C)NC[C@H](O)c1cccnc1C(C)(F)F] | 259.3 |
| 03-84 | 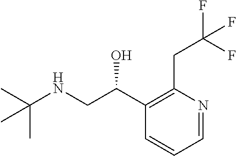 [SMILES: CC(C)(C)NC[C@H](O)c1cccnc1CC(F)(F)F] | 277.3 |
| 03-85 | 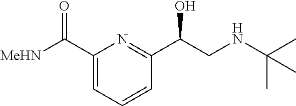 [SMILES: CNC(=O)c1cccc([C@@H](O)CNC(C)(C)C)n1] | 252.2 |
| 03-86 | 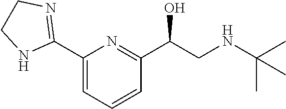 [SMILES: CC(C)(C)NC[C@H](O)c1cccc(C2=NCCN2)n1] | 263.1 |
| 03-87 | 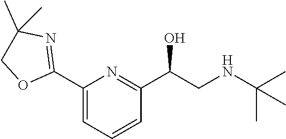 [SMILES: CC1(C)COC(c2cccc([C@@H](O)CNC(C)(C)C)n2)=N1] | |
| 03-88 | 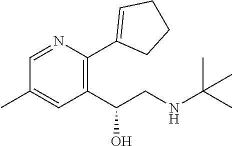 [SMILES: Cc1cnc(C2=CCCC2)c([C@@H](O)CNC(C)(C)C)c1] | 275.3 |
| 03-89 | 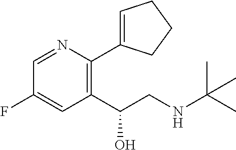 [SMILES: CC(C)(C)NC[C@H](O)c1cc(F)cnc1C1=CCCC1] | 279.4 |
| 03-90 | 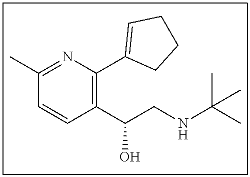 [SMILES: Cc1ccc([C@@H](O)CNC(C)(C)C)c(C2=CCCC2)n1] | 275.4 |
| 03-91 | 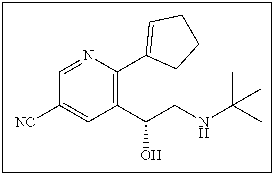 [SMILES: CC(C)(C)NC[C@H](O)c1cc(C#N)cnc1C1=CCCC1] | |
| 03-92 | 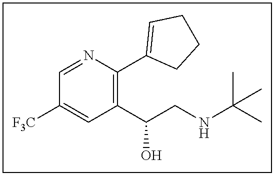 [SMILES: CC(C)(C)NC[C@H](O)c1cc(C(F)(F)F)cnc1C1=CCCC1] | 329.4 |
| 03-93 | 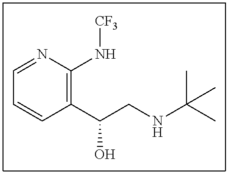 [SMILES: CC(C)(C)NC[C@H](O)c1cccnc1NC(F)(F)F] | |
| 03-94 | 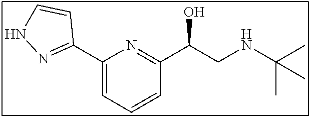 [SMILES: CC(C)(C)NC[C@H](O)c1cccc(-c2cc[nH]n2)n1] | |
| 03-95 | 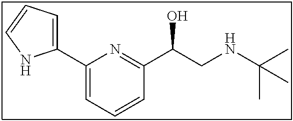 [SMILES: CC(C)(C)NC[C@H](O)c1cccc(-c2ccc[nH]2)n1] | 260.2 |
| 03-96 | 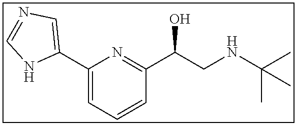 [SMILES: CC(C)(C)NC[C@H](O)c1cccc(-c2cnc[nH]2)n1] | |
| 03-97 | 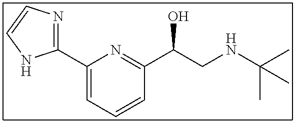 [SMILES: CC(C)(C)NC[C@H](O)c1cccc(-c2ncc[nH]2)n1] | |
| 03-98 | 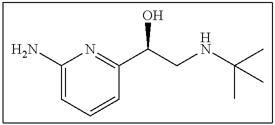 | 210.1 |
| 03-99 | 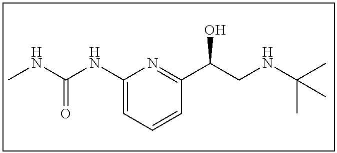 [SMILES: CNC(=O)Nc1cccc([C@@H](O)CNC(C)(C)C)n1] | |
| 03-100 | 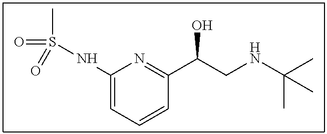 [SMILES: CC(C)(C)NC[C@H](O)c1cccc(NS(C)(=O)=O)n1] | |
| 03-101 | 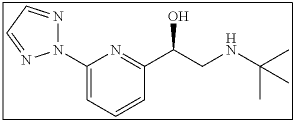 [SMILES: CC(C)(C)NC[C@H](O)c1cccc(-n2nccn2)n1] | 262.2 |
| 03-102 | 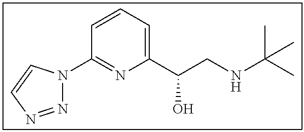 [SMILES: CC(C)(C)NC[C@H](O)c1cccc(-n2ccnn2)n1] | 262.2 |
| 03-103 | 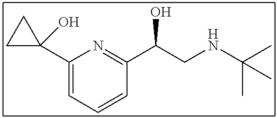 [SMILES: CC(C)(C)NC[C@H](O)c1cccc(C2(O)CC2)n1] | |
| 03-104 | 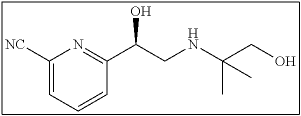 [SMILES: CC(C)(CO)NC[C@H](O)c1cccc(C#N)n1] | 236.2 |
| 03-105 | 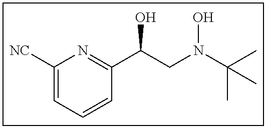 [SMILES: CC(C)(C)N(O)C[C@H](O)c1cccc(C#N)n1] | |
| 03-106 | 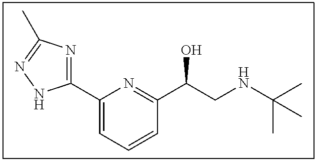 [SMILES: Cc1n[nH]c(-c2cccc([C@@H](O)CNC(C)(C)C)n2)n1] | |
| 03-107 | 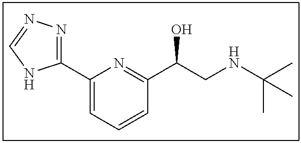 [SMILES: CC(C)(C)NC[C@H](O)c1cccc(-c2nnc[nH]2)n1] | |
| 03-108 | 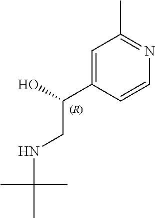 | 209.2 |
| 03-109 | 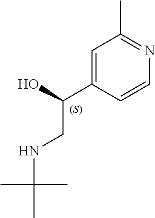 | 209.2 |
| 03-110 | 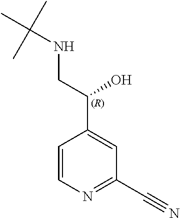 | 220.2 |
| 03-111 | 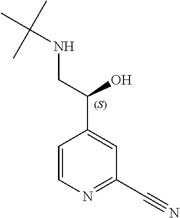 | 220.2 |
| 03-112 | 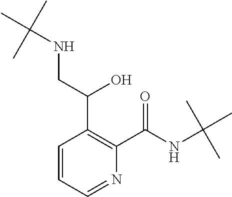 [SMILES: CC(C)(C)NCC(O)c1cccnc1C(=O)NC(C)(C)C] | 294.2 |
| 03-113 | 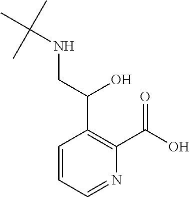 [SMILES: CC(C)(C)NCC(O)c1cccnc1C(=O)O] | 239.23 |
| 03-114 | 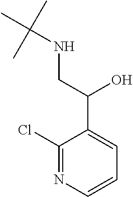 | 229.1 |
| 03-115 | 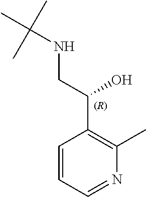 | 209.2 |
| 03-116 | 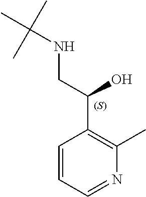 | 209.2 |
| 03-117 | 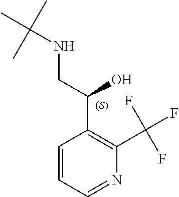 | 263.2 |
| 03-118 | 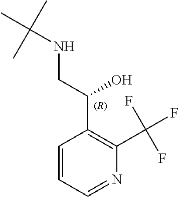 | 263.2 |
| 03-119 | 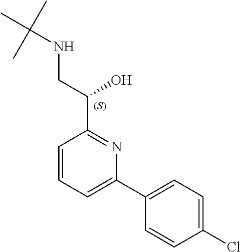 | 305.37 |
| 03-120 | 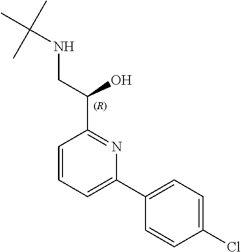 | 305.37 |
| 03-121 | 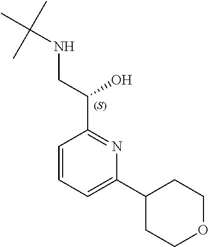 | 279.3 |
| 03-122 | 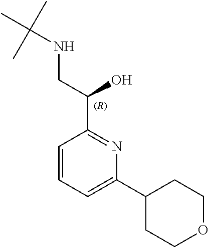 | 279.3 |
| 03-123 | 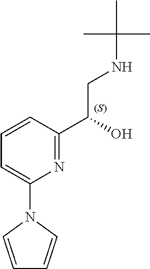 | 260.27 |
| 03-124 | 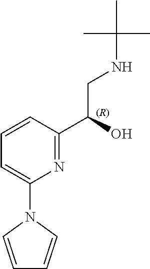 | 260.23 |
| 03-125 | 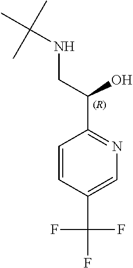 | 263.1 |
| 03-126 | 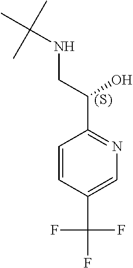 | 263.1 |
| 03-127 | 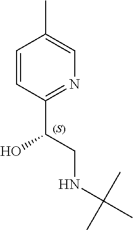 | 209.1 |
| 03-128 | 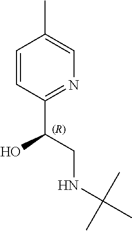 | 209.1 |
| 03-129 | 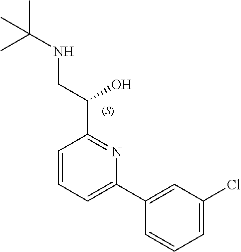 | 305.32 |
| 03-130 | 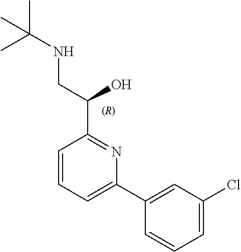 | 305.35 |
| 03-131 | 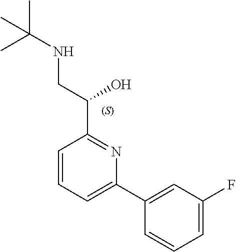 | 289.35 |
| 03-132 | 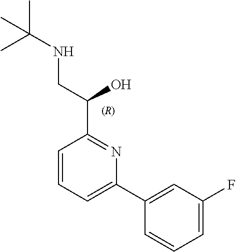 | 289.32 |
| 03-133 | 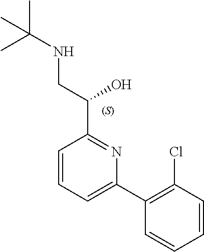 | 305.28 |
| 03-134 | 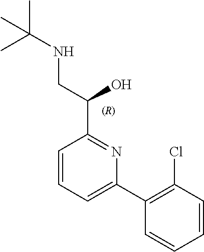 | 305.28 |
| 03-135 | 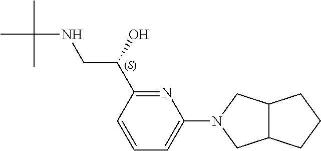 | 304.4 |
| 03-136 | 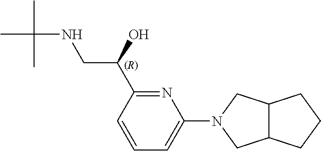 | 304.4 |
| 03-137 | 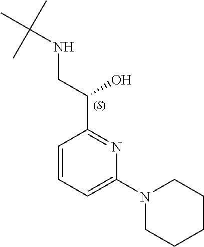 | 278.41 |
| 03-138 | 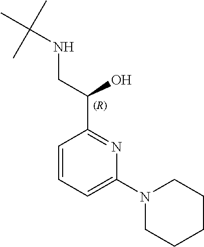 | 278.41 |
| 03-139 | 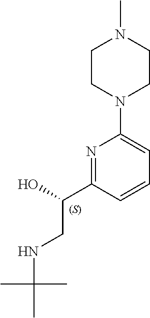 | 293.42 |
| 03-140 | 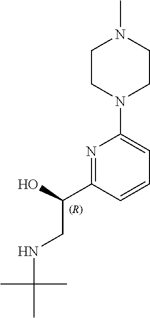 | 293.42 |
| 03-141 | 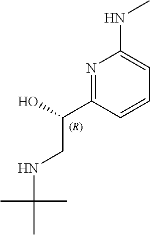 | 224.25 |
| 03-142 | 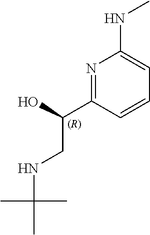 | 224.25 |
| 03-143 | 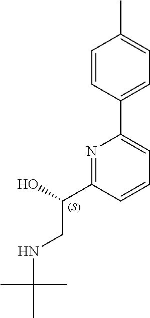 | 285.22 |
| 03-144 | 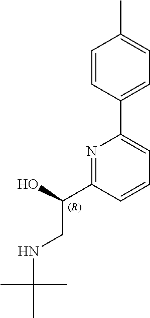 | 285.22 |
| 03-145 | 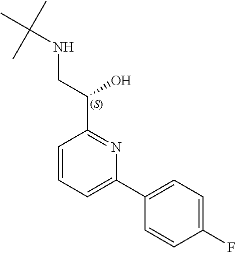 | 289.38 |
| 03-146 | 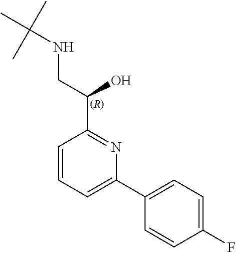 | 289.38 |
| 03-147 | 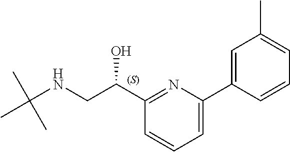 | 285.35 |
| 03-148 | 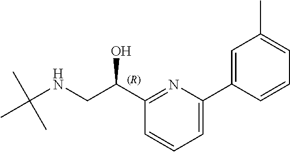 | 285.35 |
| 03-149 | 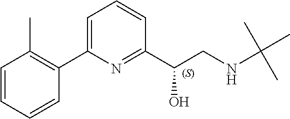 | 285.33 |
| 03-150 | 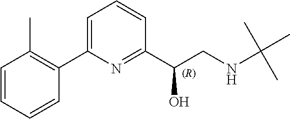 | 285.33 |
| 03-151 | 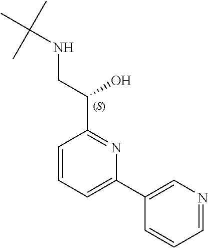 | 272.33 |
| 03-152 | 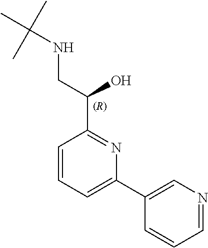 | 272.33 |
| 03-153 | 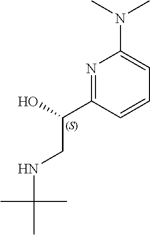 | 238.1 |
| 03-154 | 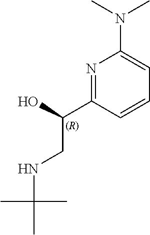 | 238.1 |
| 03-155 | 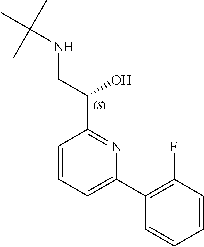 | 289.29 |
| 03-156 | 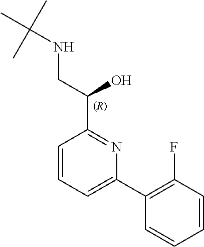 | 289.29 |
| 03-157 | 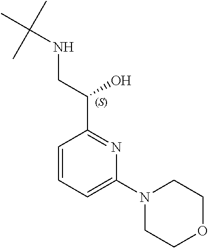 | 280.32 |
| 03-158 | 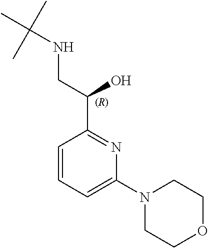 | 280.32 |
| 03-159 | 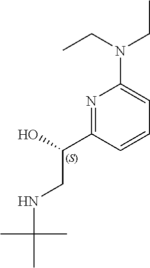 | 266.32 |
| 03-160 | 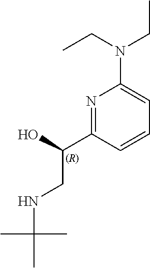 | 266.32 |
| 03-161 | 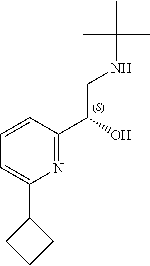 | 249.18 |
| 03-162 | 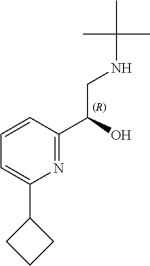 | 249.18 |
| 03-163 | 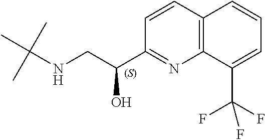 | 313.26 |
| 03-164 | 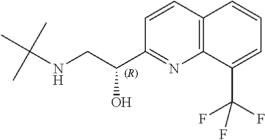 | 313.26 |
| 03-165 | 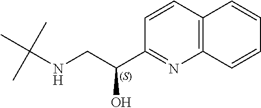 | 245.1 |
| 03-166 | 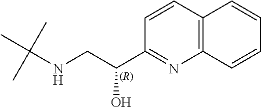 | 245.1 |
| 03-167 | 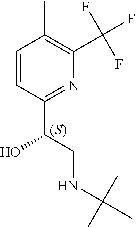 | 277.1 |
| 03-168 | 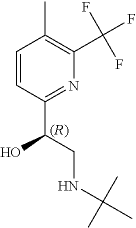 | 277.1 |
| 03-169 | 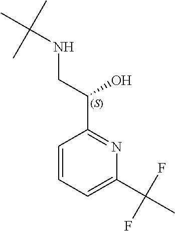 | 259.13 |
| 03-170 | 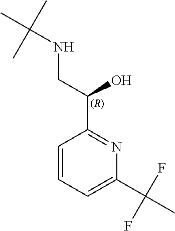 | 259.13 |
| 03-171 | 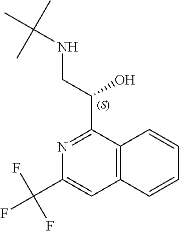 | 313.28 |
| 03-172 | 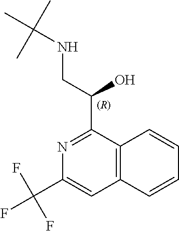 | 313.28 |
| 03-173 | 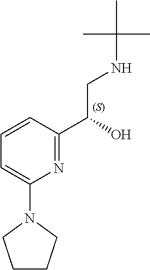 | 264.3 |
| 03-174 | 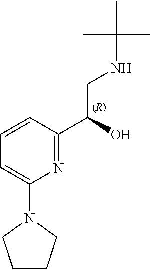 | 264.3 |
| 03-175 | 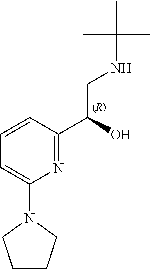 | 237.28 |
| 03-176 | 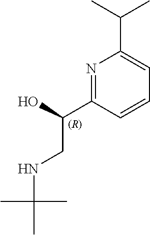 | 237.28 |
| 03-177 | 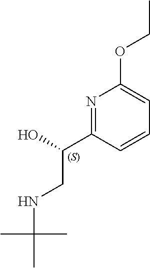 | 239.26 |
| 03-178 | 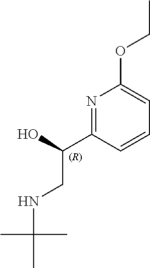 | 239.26 |
| 03-179 | 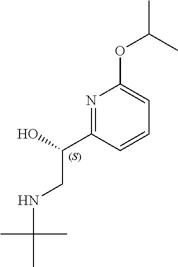 | 253.2 |
| 03-180 | 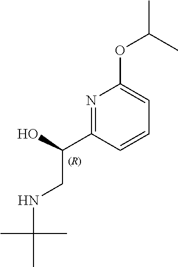 | 253.2 |
| 03-181 | 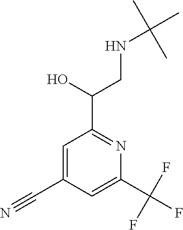 [SMILES: CC(C)(C)NCC(O)c1cc(C#N)cc(C(F)(F)F)n1] | 288.23 |
| 03-182 | 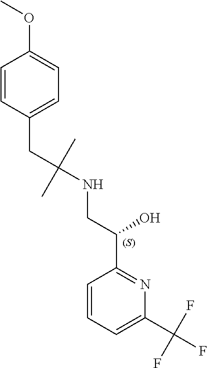 | 369.26 |
| 03-183 | 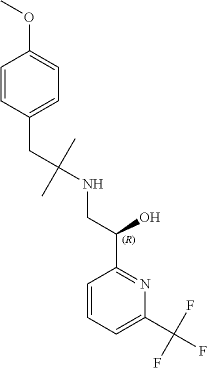 | 369.26 |
| 03-184 | 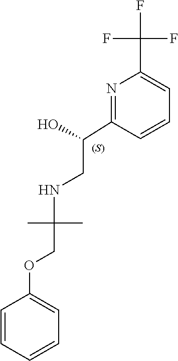 | 355.32 |
| 03-185 | 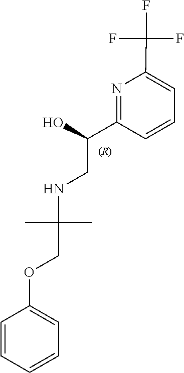 | 355.32 |
| 03-186 | 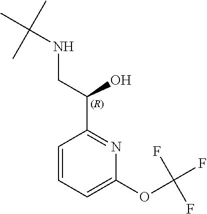 | 279.17 |
| 03-187 | 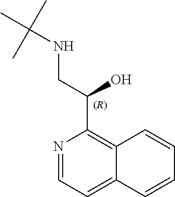 | 245.1 |
| 03-188 | 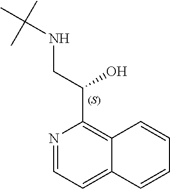 | 245.1 |
| 03-189 | 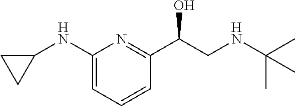 [SMILES: CC(C)(C)NC[C@H](O)c1cccc(NC2CC2)n1] | 250.2 |
| 03-190 | 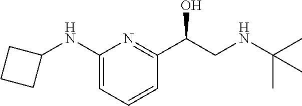 [SMILES: CC(C)(C)NC[C@H](O)c1cccc(NC2CCC2)n1] | 264.3 |
| 03-191 | 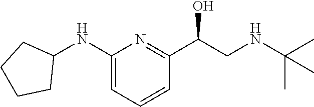 [SMILES: CC(C)(C)NC[C@H](O)c1cccc(NC2CCCC2)n1] | 278.4 |
| 03-192 | 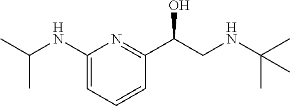 [SMILES: CC(C)Nc1cccc([C@@H](O)CNC(C)(C)C)n1] | 252.3 |
| 03-193 | 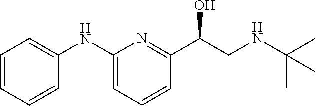 [SMILES: CC(C)(C)NC[C@H](O)c1cccc(Nc2ccccc2)n1] | 286.3 |
| 03-194 | 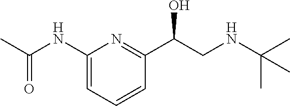 [SMILES: CC(=O)Nc1cccc([C@@H](O)CNC(C)(C)C)n1] | 252.3 |
| 03-195 | 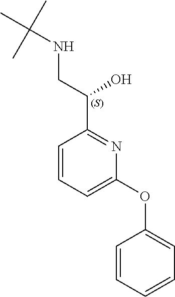 | 287.2 |
| 03-196 | 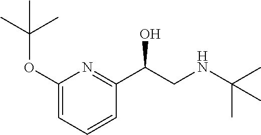 [SMILES: CC(C)(C)NC[C@H](O)c1cccc(OC(C)(C)C)n1] | 267.2 |
| 03-197 | 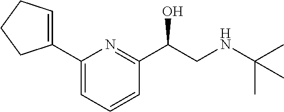 [SMILES: CC(C)(C)NC[C@H](O)c1cccc(C2=CCCC2)n1] | 261.3 |
| 03-198 | 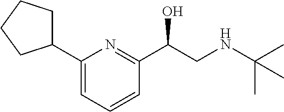 [SMILES: CC(C)(C)NC[C@H](O)c1cccc(C2CCCC2)n1] | 263.3 |
| 03-199 | 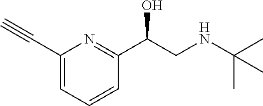 [SMILES: C#Cc1cccc([C@@H](O)CNC(C)(C)C)n1] | 219.2 |
| 03-200 | 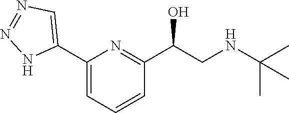 [SMILES: CC(C)(C)NC[C@H](O)c1cccc(-c2cnn[nH]2)n1] | 262.2 |
| 03-201 | 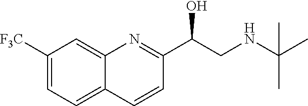 [SMILES: CC(C)(C)NC[C@H](O)c1ccc2ccc(C(F)(F)F)cc2n1] | |
| 03-202 | 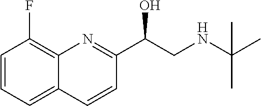 [SMILES: CC(C)(C)NC[C@H](O)c1ccc2cccc(F)c2n1] | |
| 03-203 | 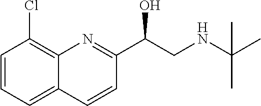 [SMILES: CC(C)(C)NC[C@H](O)c1ccc2cccc(Cl)c2n1] | |
| 03-204 | 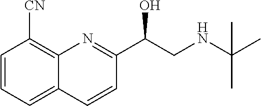 [SMILES: CC(C)(C)NC[C@H](O)c1ccc2cccc(C#N)c2n1] | |
| 03-205 | 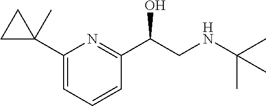 [SMILES: CC(C)(C)NC[C@H](O)c1cccc(C2(C)CC2)n1] | |
| 03-206 | 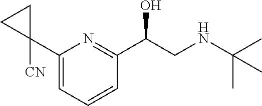 [SMILES: CC(C)(C)NC[C@H](O)c1cccc(C2(C#N)CC2)n1] | 260.4 |
| 03-207 | 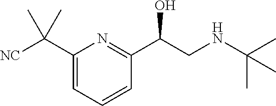 [SMILES: CC(C)(C)NC[C@H](O)c1cccc(C(C)(C)C#N)n1] | 262.3 |
| 03-208 | 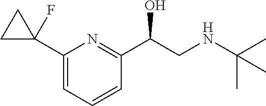 [SMILES: CC(C)(C)NC[C@H](O)c1cccc(C2(F)CC2)n1] | |
| 03-209 | 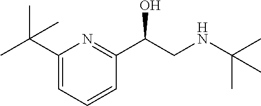 [SMILES: CC(C)(C)NC[C@H](O)c1cccc(C(C)(C)C)n1] | 251.2 |
| 03-210 | 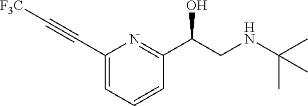 [SMILES: CC(C)(C)NC[C@H](O)c1cccc(C#CC(F)(F)F)n1] | |
| 03-211 | 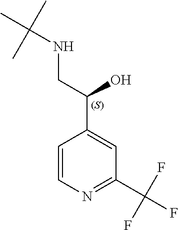 | 263.2 |
| 03-212 | 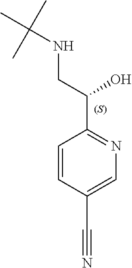 | 220.2 |
| 03-213 | 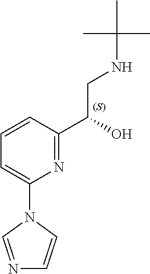 | 261.3 |
| 03-214 | 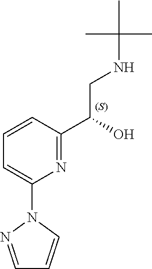 | 261.2 |
| 03-215 | 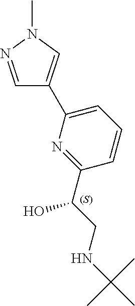 | 275.3 |
| 03-216 | 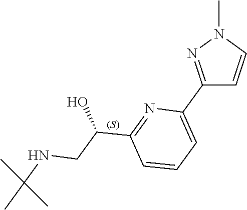 | 275.3 |
| 03-217 | 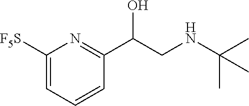 | |
| 03-218 | 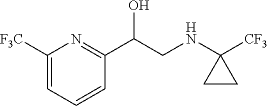 [SMILES: OC(CNC1(C(F)(F)F)CC1)c1cccc(C(F)(F)F)n1] | 315.2 |
| 03-219 | 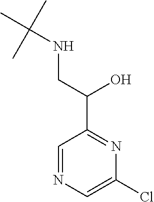 | 230.4 |
| 03-220 | 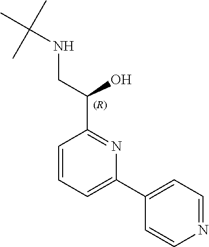 | 272.5 |
| 03-221 | 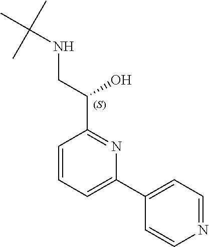 | 272.5 |
| 03-222 | 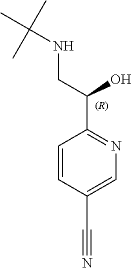 | 220.2 |
| 03-224 | 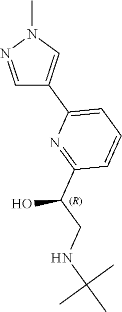 | 275.3 |
| 03-225 | 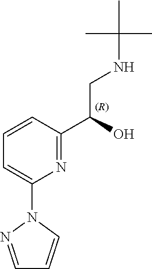 | 261.2 |
| 03-226 | 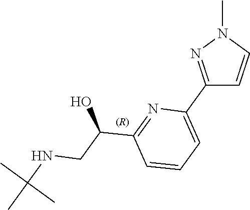 | 275.3 |
| 03-227 | 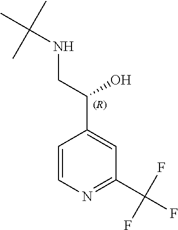 | 263.2 |
| 03-228 | 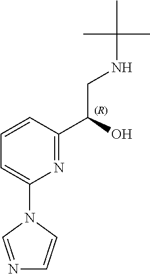 | 261.3 |
| 03-229 | 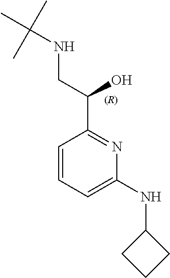 | 264.3 |
| 03-230 | 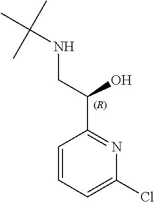 | 229.5 |
| 03-231 | 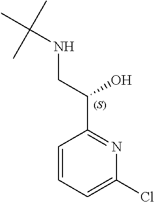 | 229.5 |
| 03-232 | 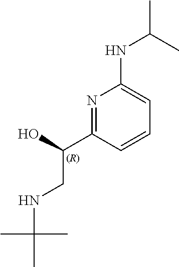 | 252.3 |
| 03-233 | 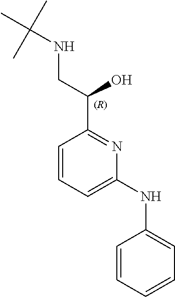 | 286.3 |
| 03-234 | 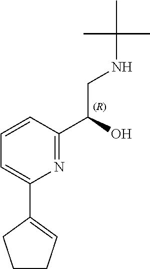 | 261.3 |
| 03-235 | 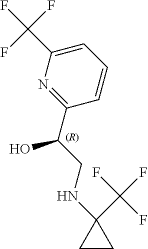 | 315.2 |
| 03-236 | 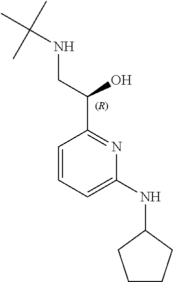 | 278.4 |
| 03-237 | 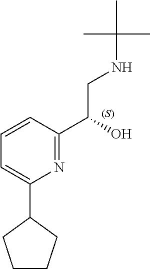 | 263.2 |
| 03-238 | 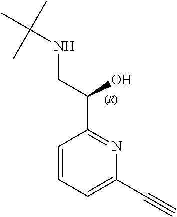 | 219.2 |
| 03-239 | 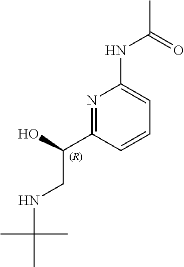 | 252.3 |
| 03-240 | 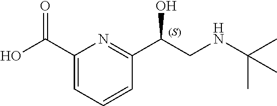 | 239.1 |
| 03-241 | 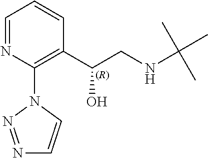 | 262.3 |
| 03-242 | 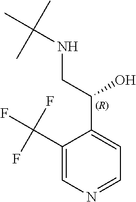 | 263.2 |
| 03-243 | 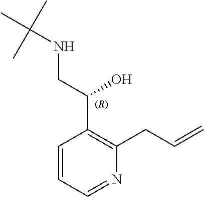 | 235.3 |
| 03-244 | 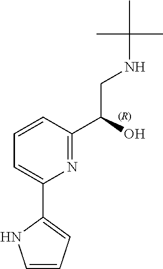 | 260.2 |
| 03-245 | 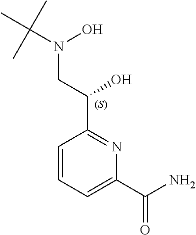 | 254.1 |
| 03-246 | 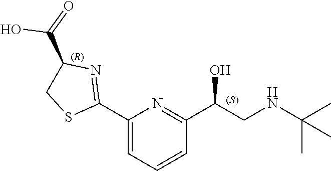 | 324.1 |
| 03-247 | 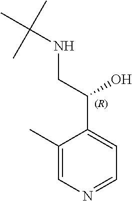 | 209.1 |
| 03-248 | 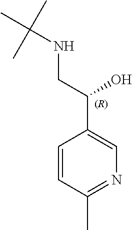 | 209.1 |
| 03-249 | 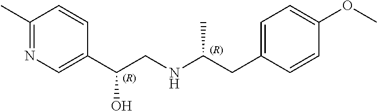 | 301.4 |
| 03-250 | 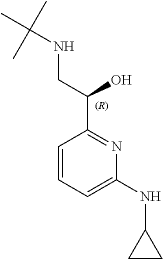 | 250.2 |
| 03-252 | 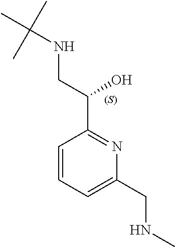 | 238.3 |
| 03-253 | 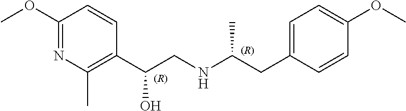 | 331.3 |
| 03-254 | 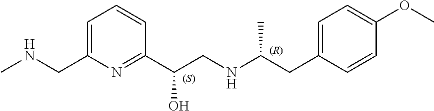 | 330.4 |
| 03-257 | 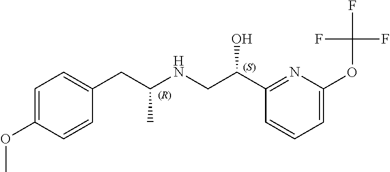 | 371.3 |
| 03-258 | 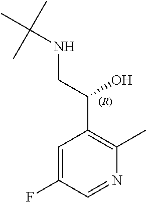 | 227.3 |
| 03-259 | 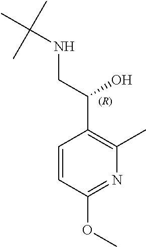 | 239.2 |
| 03-260 | 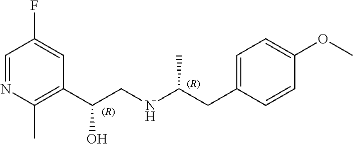 | 319.4 |
| 03-261 | 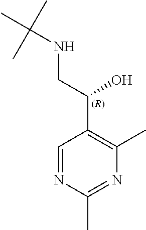 | 224.3 |
| 03-262 | 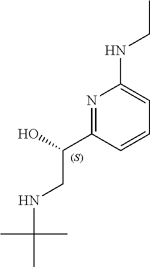 | 238.3 |
| 03-263 | 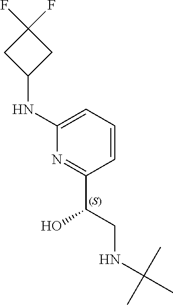 | 300.3 |
| 03-264 | 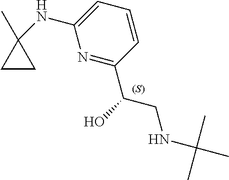 | 264.3 |
| 03-265 | 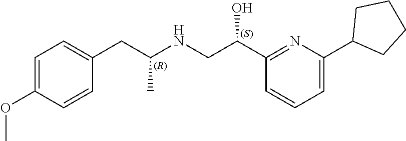 | 355.5 |
| 03-266 | 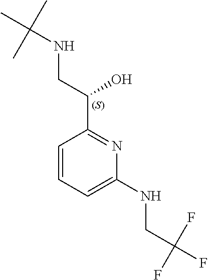 | 292.3 |
| 03-267 | 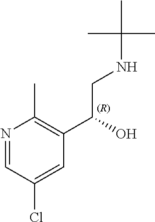 | 243.6 |
| 03-268 | 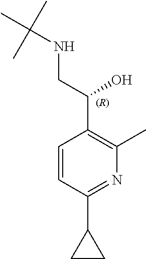 | 249.3 |
| 03-269 | 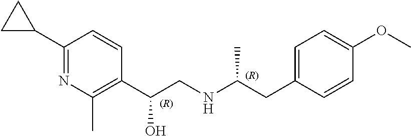 | 341.5 |
| 03-270 | 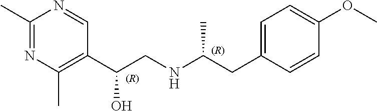 | 316.4 |
| 03-271 | 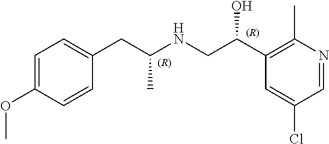 | 335.8 |
| 03-272 | 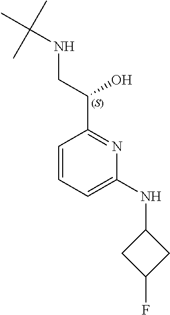 | 282.2 |
| 03-273 | 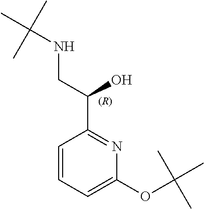 | 267.2 |
| 03-274 | 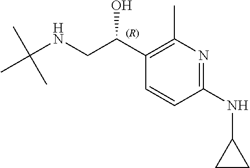 | 264.3 |
| 03-275 | 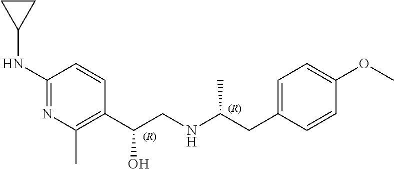 | 356.4 |
| 03-276 | 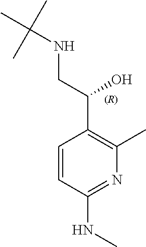 | 238.3 |
| 03-277 | 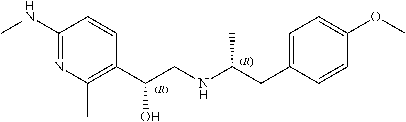 | 330.4 |
| 03-278 | 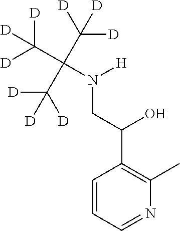 [SMILES: [2H]C([2H])([2H])C(NCC(O)c1cccnc1C)(C([2H])([2H])[2H])C([2H])([2H])[2H]] | 218.3 |
| 03-279 | 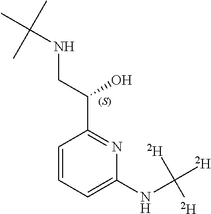 | 227.2 |
| 03-280 | 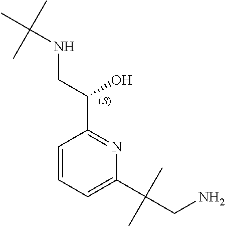 | 266.3 |
| 03-281 | 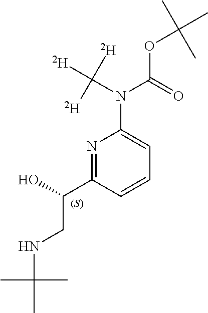 | 327.3 |

In some embodiments, a β-agent is an optically pure stereoisomer, pharmaceutically acceptable salt, solvate, or prodrug of a compound of Table 1.

### EXAMPLES

The present disclosure will be further described in the following examples, which do not limit the scope of the present disclosure.

### Example 1: Preparation of Pure Nadolol Isomer

2.09 g of Nadolol (sourced from Sigma-Aldrich) was separated via supercritical fluid chromatography (SFC) to afford 0.345 g NI-Cas an off-white solid, 0.370 g NI-D as an off-white solid, 0.350 g NI-A as an off-white solid and 0.300 g NI-B as an off-white solid;

(NI-C): mp=132-136° C.; 1H NMR (400 MHz, DMSO-d6) d 7.02 (dd, t, J=7.8 Hz, 1H), 6.87 (d, J=8.0 Hz, 1H), 6.62 (d, J=7.6 Hz, 1H), 4.85 (d, J=4.0 Hz, 1H), 4.56 (dd J=5.8, 4.2 Hz, 2H), 3.90-3.73 (m, 5H), 2.85-2.51 (m, 6H), 1.40-1.30 (bs, 1H), 1.03 (s, 9H); LCMS [M+H]+=310.38; SFC (ChiralPak AD-H(4.6*250) 5 u; 1 ml/min isocratic, 4:1 n-hexane:EtOH with 0.3% DEA) rt=11.085 (220 nm).

(NI-D): mp=133-137° C.; 1H NMR (400 MHz, DMSO-d6) d 7.02 (dd, t, J=7.8 Hz, 1H), 6.69 (d, J=8.0 Hz, 1H), 6.62 (d, J=7.6 Hz, 1H), 4.65 (bs, 1H), 4.56 (t, J=4.4 Hz, 2H), 3.91-3.71 (m, 5H), 2.81-2.53 (m, 6H), 1.36 (bs, 1H), 1.02 (s, 9H); LCMS [M+H]+=310.38; SFC (ChiralPak AD-H(4.6*250) 5 u; 1 ml/min isocratic, 4:1 n-hexane:EtOH with 0.3% DEA) rt=33.38 (220 nm).

(NI-A): mp=145-149° C.; 1H NMR (400 MHz, DMSO-d6)

7.02 (dd, t, J=7.8 Hz, 1H), 6.69 (d, J=8.0 Hz, 1H), 6.62 (d, J=7.6 Hz, 1H), 4.86 (bs, 1H), 4.56 (t, J=4.4 Hz, 2H), 3.91-3.75 (m, 5H), 2.82-2.51 (m, 6H), 1.42 (bs, 1H), 1.04 (s, 9H); LCMS [M+H]+=310.38; S SFC (ChiralPak AD-H(4.6*250) 5 u; 1 ml/min isocratic, 4:1 n-hexane:EtOH with 0.3% DEA) rt=9.21 (220 nm).

(NI-B): mp=143-147° C.; 1H NMR (400 MHz, DMSO-d6) d 7.02 (dd, t, J=7.8 Hz, 1H), 6.69 (d, J=7.6 Hz, 1H), 6.63 (d, J=7.2 Hz, 1H), 4.90 (bs, 1H), 4.56 (dd, J=5.8, 4.2 Hz, 2H), 3.90-3.75 (m, 5H), 2.81-2.51 (m, 6H), 1.51 (bs, 1H), 1.04 (s, 9H); LCMS [M+H]+=310.38; SFC (ChiralPak AD-H(4.6*250) 5 u; 1 ml/min isocratic, 4:1 n-hexane:EtOH with 0.3% DEA) rt=18.81 (220 nm).

### Example 2: Structure Determination of Ni-D

2 mg NI-D was dissolved in 0.2 mL acetonitrile/methyl tert-butyl ether (1:1) and kept in a half sealed 4 mL vial. The solution evaporates slowly at room temperature. Crystals were observed on the second day.

The crystal was a colourless block with the following dimensions: 0.10×0.10×0.10 mm3. The symmetry of the crystal structure was assigned the monoclinic space group P21 with the following parameters: a=10.90210(10) Å, b=25.5523(3) Å, c=12.0458(2) Å, α=90°, β=95.4270(10)°, γ=90°, V=3340.60(7) Å3, Z=8, Dc=1.230 g/cm3, F(000)=1344.0, μ(CuKα)=0.703 mm-1, and T=150.00(10) K.

A total of 52904 reflections were collected in the 20 range from 6.918 to 133.194 (Rigaku Oxford Diffraction XtaLAB Synergy four-circle diffractometer equipped with a HyPix-6000HE area detector; Cryogenic system: Oxford Cryostream 800; Cu: λ=1.54184 Å, 50 W, Micro focus source with multilayer mirror (μ-CMF); Distance from the crystal to the CCD detector: d=35 mm; Tube Voltage: 50 kV; Tube Current: 1 mA). The limiting indices were: −12≤h≤12, −29≤k≤30, −14≤1≤14; which yielded 10655 unique reflections (Rint=0.0563). The structure was solved using SHELXT (Sheldrick, G. M. 2015. Acta Cryst. A71, 3-8) and refined using SHELXL (against F²) (Sheldrick, G. M. 2015. Acta Cryst. C71, 3-8). The total number of refined parameters was 829, compared with 10655 data. All reflections were included in the refinement. The goodness of fit on F² was 1.052 with a final R value for [I>2σ(I)]R1=0.0336 and wR2=0.0891. The largest differential peak and hole were 0.29 and −0.37 Å-3, respectively.

Structure was confirmed to be consistent with (SSR)-nadolol.

### Data Tables

TABLE 2
Summary of X-ray Crystallographic Data

| | |
|---|---|
| Crystal size/mm³ | 0.10 × 0.10 × 0.10 |
| Radiation Type | CuKα (λ = 1.54184) |
| Crystal system | monoclinic |
| Space group | P2₁ |
| a/Å | 10.90210(10) |
| b/Å | 25.5523(3) |
| c/Å | 12.0458(2) |
| α/° | 90 |
| β/° | 95.4270(10) |
| γ/° | 90 |
| Cell Volume/Å³ | 3340.60(7) |
| Cell Formula Units Z | 8 |
| Crystal Density calc g/cm³ | 1.230 |
| Crystal F(000) | 1344.0 |
| Absorption Coefficient μ/mm⁻¹ | 0.703 |
| Index ranges | −12 ≤ h ≤ 12, −29 ≤ |
| | k ≤ 30, −14 ≤ 1 ≤ 14 |
| Cell Measurement Temperature/K | 150.00(10) |
| 2θ range for data collection/° | 6.918 to 133.194 |
| Goodness-of-fit on F² | 1.052 |
| Final R indexes [I >= 2σ (I)] | R₁ = 0.0336, wR₂ = 0.0891 |
| Final R indexes [all data] | R₁ = 0.0354, wR₂ = 0.0907 |
| Largest diff. peak/hole/e Å⁻³ | 0.29/−0.37 |
| Reflections collected/unique | 52904/10655 [Rᵢₙₜ = 0.0563] |
| Flack parameter | −0.01(7) |

TABLE 3
Atomic coordinates (×10\hat{ }4) and equivalent isotropic displacement
parameters (A\hat{ }2 × 10\hat{ }3).

| Atom | x | y | z | U(eq) |
|---|---|---|---|---|
| O(11) | 2188.5(13) | 4350.8(6) | 10032.1(13) | 23.8(3) |
| O(3) | 3204.3(16) | 7876.3(6) | 6779.5(14) | 30.6(4) |
| O(7) | 7367.0(14) | 4421.4(6) | 10050.4(13) | 27.9(4) |
| O(4) | 2875.6(15) | 6877.9(6) | 7475.0(15) | 30.1(4) |
| O(8) | 10064.7(13) | 3597.2(6) | 10231.6(14) | 26.7(3) |
| O(6) | 5570.6(13) | 5732.4(6) | 7752.5(14) | 26.9(4) |
| O(1) | 2342.3(14) | 8542.2(7) | 2911.2(13) | 29.9(4) |
| O(10) | 544.5(13) | 5705.0(7) | 7737.7(15) | 29.5(4) |
| O(15) | 1479.0(17) | 2869.2(7) | 3339.6(15) | 34.7(4) |
| O(2) | 24.5(14) | 8022.4(7) | 3518.8(15) | 32.7(4) |
| O(5) | 2957.9(14) | 5818.6(7) | 7887.7(17) | 37.9(5) |
| O(16) | 1826.5(15) | 1930.9(7) | 2472.8(15) | 33.2(4) |
| O(14) | 2650.3(14) | 3237.9(8) | 6937.9(13) | 30.9(4) |
| O(13) | 5106.9(15) | 3791.8(8) | 7241.7(14) | 38.7(4) |
| N(1) | 4495.4(16) | 6377.1(7) | 6079.0(15) | 20.5(4) |
| O(9) | −2014.2(15) | 5901.3(8) | 8050(2) | 45.7(5) |
| N(4) | 420.6(16) | 1356.8(7) | 3925.8(15) | 21.1(4) |
| N(3) | 3299.1(17) | 2810.3(8) | 8995.2(16) | 22.8(4) |
| N(2) | 8358.4(16) | 2922.1(7) | 8933.7(16) | 20.8(4) |
| C(43) | 885.0(19) | 5084.7(8) | 9885.9(18) | 19.6(4) |
| C(26) | 5882.2(19) | 5086.4(8) | 9879.7(18) | 20.3(4) |
| C(44) | 1824(2) | 4806.0(8) | 10519.3(18) | 20.3(4) |
| O(12) | 4929.6(17) | 3605.2(8) | 9778(3) | 70.7(9) |
| C(12) | 4088(2) | 7068.6(9) | 7415.8(18) | 25.4(5) |
| C(29) | 8830.5(18) | 3749.9(9) | 9879.0(18) | 20.6(4) |
| C(27) | 6834(2) | 4834.5(9) | 10554.3(19) | 22.5(4) |
| C(45) | 3332(2) | 4120.4(9) | 10480(2) | 25.9(5) |
| C(28) | 8393.4(19) | 4166.6(9) | 10659.4(18) | 22.8(5) |
| C(32) | 8243(2) | 2052.1(9) | 9897(2) | 28.5(5) |
| C(35) | 2312(2) | 4980.9(10) | 11554(2) | 28.5(5) |
| C(18) | 7185(2) | 5002(1) | 11625(2) | 30.0(5) |
| C(9) | 2556(2) | 8547.4(9) | 5539.9(18) | 22.0(4) |
| C(61) | 1523(2) | 3380.4(9) | 3642.1(19) | 26.5(5) |
| C(10) | 3339(2) | 8388.1(9) | 6476(2) | 25.1(5) |
| C(23) | 3731(2) | 5485.1(9) | 8606(2) | 27.5(5) |
| C(42) | 444.8(19) | 4899.0(9) | 8725.8(19) | 22.7(4) |
| C(25) | 5571(2) | 4905.9(9) | 8690.4(18) | 22.4(4) |
| C(21) | 5248(2) | 5492.3(9) | 10340(2) | 23.9(5) |
| C(60) | 2404(2) | 3487.2(9) | 4548.1(18) | 24.8(5) |
| C(55) | 2557(2) | 3996.8(9) | 4926(2) | 28.3(5) |
| C(31) | 7808.4(19) | 2388.5(9) | 8880.2(19) | 22.9(5) |
| C(4) | 2676(2) | 9050.6(9) | 5138.3(19) | 25.5(5) |
| C(38) | 403(2) | 5527.9(9) | 10355(2) | 25.1(5) |
| C(41) | −291.3(19) | 5313.4(9) | 8044(2) | 25.9(5) |
| C(62) | 557(2) | 2679.8(9) | 2518.3(19) | 29.1(5) |
| C(48) | 2824.6(19) | 2267.7(9) | 9079(2) | 25.1(5) |
| C(59) | 3123(2) | 3035.8(9) | 5093(2) | 28.9(5) |
| C(49) | 3423(2) | 1980.2(10) | 10103(2) | 29.0(5) |
| C(30) | 8057.3(19) | 3258.3(9) | 9855.7(18) | 22.1(4) |
| C(64) | 205(2) | 1917.8(9) | 3730.9(19) | 26.1(5) |
| C(11) | 4091(2) | 7655.3(9) | 7596.1(19) | 28.8(5) |
| C(40) | −1256(2) | 5548.2(9) | 8731(2) | 31.5(6) |
| C(13) | 4569(2) | 6940.1(9) | 6307(2) | 28.7(5) |
| C(47) | 3018(2) | 3183.6(9) | 9856.0(19) | 24.3(5) |
| C(3) | 3542(2) | 9394.4(10) | 5672(2) | 34.7(6) |
| C(15) | 4564(2) | 5639.9(10) | 4834(2) | 32.5(5) |
| C(36) | 1845(3) | 5432.4(11) | 11987(2) | 37.5(6) |
| C(33) | 8193(2) | 2134.4(10) | 7821(2) | 27.5(5) |
| C(52) | 792(2) | 3773.4(10) | 3139(2) | 32.6(5) |
| C(46) | 3645(2) | 3694.5(9) | 9665(2) | 30.1(5) |
| C(6) | 1348(2) | 8791.2(10) | 3402.9(18) | 27.7(5) |
| C(1) | 4181(2) | 8732.5(10) | 7016(2) | 36.2(6) |
| C(58) | 3658(2) | 3174.4(10) | 6265(2) | 28.7(5) |
| C(7) | 695(2) | 8419.4(9) | 4145.1(19) | 25.9(5) |
| C(66) | 531(2) | 597.6(11) | 5129(2) | 35.7(6) |
| C(56) | 3506(2) | 4127.8(10) | 5898(2) | 33.7(6) |
| C(20) | 5585(2) | 5649.7(10) | 11431(2) | 33.1(6) |
| C(37) | 889(3) | 5697.2(10) | 11405(2) | 36.0(6) |
| C(5) | 1881(2) | 9240.4(9) | 4118(2) | 31.2(5) |
| C(51) | 1429(2) | 2290.5(11) | 9114(2) | 36.3(6) |
| C(50) | 3111(2) | 1982.9(10) | 8020(2) | 31.4(5) |
| C(22) | 4203(2) | 5773.4(9) | 9663(2) | 30.4(5) |
| C(57) | 4387(2) | 3678.2(10) | 6202(2) | 30.4(5) |
| C(53) | 943(3) | 4280.6(10) | 3543(2) | 38.9(6) |
| C(19) | 6554(3) | 5415.3(11) | 12057(2) | 35.9(6) |
| C(54) | 1816(3) | 4393.2(10) | 4421(2) | 36.9(6) |
| C(34) | 6411(2) | 2439.4(10) | 8792(2) | 34.2(6) |
| C(68) | 609(2) | 1485.0(12) | 6009(2) | 34.2(6) |
| C(17) | 4397(2) | 6535.8(11) | 4011(2) | 34.1(6) |
| C(39) | −634(2) | 5834.8(10) | 9728(2) | 33.4(6) |
| C(67) | −1338(2) | 1157.1(13) | 4971(2) | 39.0(6) |
| C(2) | 4280(2) | 9233.9(10) | 6604(2) | 39.6(6) |
| C(16) | 6340(2) | 6248.8(13) | 5109(2) | 39.6(6) |

TABLE 4
Bond lengths [A]

| Atom | Atom | Length/Å | Atom | Atom | Length/Å |
|---|---|---|---|---|---|
| O(11) | C(44) | 1.378(3) | C(65) | C(68) | 1.531(3) |
| O(11) | C(45) | 1.437(3) | C(65) | C(67) | 1.530(3) |
| O(3) | C(10) | 1.369(3) | C(8) | C(9) | 1.517(3) |
| O(3) | C(11) | 1.429(3) | C(8) | C(7) | 1.522(3) |
| O(7) | C(27) | 1.374(3) | C(63) | C(62) | 1.514(3) |
| O(7) | C(28) | 1.435(3) | C(63) | C(64) | 1.520(3) |
| O(4) | C(12) | 1.417(3) | C(24) | C(23) | 1.516(3) |
| O(8) | C(29) | 1.427(3) | C(24) | C(25) | 1.519(3) |
| O(6) | C(24) | 1.433(3) | C(35) | C(36) | 1.383(4) |
| O(1) | C(6) | 1.432(3) | C(18) | C(19) | 1.388(4) |
| O(10) | C(41) | 1.425(3) | C(9) | C(10) | 1.408(3) |
| O(15) | C(61) | 1.356(3) | C(9) | C(4) | 1.384(3) |
| O(15) | C(62) | 1.426(3) | C(61) | C(60) | 1.410(3) |
| O(2) | C(7) | 1.424(3) | C(61) | C(52) | 1.385(3) |
| O(5) | C(23) | 1.430(3) | C(10) | C(1) | 1.389(3) |
| O(16) | C(63) | 1.420(3) | C(23) | C(22) | 1.518(4) |
| O(14) | C(58) | 1.434(3) | C(42) | C(41) | 1.522(3) |
| O(13) | C(57) | 1.443(3) | C(21) | C(20) | 1.390(4) |
| N(1) | C(14) | 1.488(3) | C(21) | C(22) | 1.518(3) |
| N(1) | C(13) | 1.465(3) | C(60) | C(55) | 1.384(3) |
| O(9) | C(40) | 1.429(3) | C(60) | C(59) | 1.509(3) |
| N(4) | C(65) | 1.493(3) | C(55) | C(56) | 1.525(3) |
| N(4) | C(64) | 1.468(3) | C(55) | C(54) | 1.398(4) |
| N(3) | C(48) | 1.487(3) | C(31) | C(33) | 1.525(3) |
| N(3) | C(47) | 1.462(3) | C(31) | C(34) | 1.523(3) |
| N(2) | C(31) | 1.488(3) | C(4) | C(3) | 1.401(3) |
| N(2) | C(30) | 1.466(3) | C(4) | C(5) | 1.515(3) |
| C(43) | C(44) | 1.410(3) | C(38) | C(37) | 1.393(4) |
| C(43) | C(42) | 1.510(3) | C(38) | C(39) | 1.517(3) |
| C(43) | C(38) | 1.391(3) | C(41) | C(40) | 1.521(4) |
| C(26) | C(27) | 1.411(3) | C(48) | C(49) | 1.528(3) |
| C(26) | C(25) | 1.513(3) | C(48) | C(51) | 1.528(3) |
| C(26) | C(21) | 1.391(3) | C(48) | C(50) | 1.527(3) |
| C(44) | C(35) | 1.382(3) | C(59) | C(58) | 1.518(3) |
| O(12) | C(46) | 1.413(3) | C(40) | C(39) | 1.512(4) |
| C(12) | C(11) | 1.515(3) | C(47) | C(46) | 1.501(3) |
| C(12) | C(13) | 1.517(3) | C(3) | C(2) | 1.381(4) |
| C(29) | C(28) | 1.526(3) | C(36) | C(37) | 1.377(4) |
| C(29) | C(30) | 1.512(3) | C(52) | C(53) | 1.388(4) |
| C(27) | C(18) | 1.378(3) | C(6) | C(7) | 1.526(3) |
| C(45) | C(46) | 1.526(3) | C(6) | C(5) | 1.517(3) |
| C(32) | C(31) | 1.534(3) | C(1) | C(2) | 1.382(4) |
| C(14) | C(15) | 1.521(4) | C(58) | C(57) | 1.519(3) |
| C(14) | C(17) | 1.530(3) | C(56) | C(57) | 1.520(4) |
| C(14) | C(16) | 1.523(3) | C(20) | C(19) | 1.376(4) |
| C(65) | C(66) | 1.517(4) | C(53) | C(54) | 1.385(4) |

TABLE 5
Bond angles [deg].

| Atom | Atom | Atom | Angle/° | Atom | Atom | Atom | Angle/° |
|---|---|---|---|---|---|---|---|
| C(44) | O(11) | C(45) | 117.45(17) | C(61) | C(60) | C(59) | 118.4(2) |
| C(10) | O(3) | C(11) | 118.63(18) | C(55) | C(60) | C(61) | 119.2(2) |
| C(27) | O(7) | C(28) | 117.70(17) | C(55) | C(60) | C(59) | 122.3(2) |
| C(61) | O(15) | C(62) | 121.21(19) | C(60) | C(55) | C(56) | 120.7(2) |
| C(13) | N(1) | C(14) | 115.39(18) | C(60) | C(55) | C(54) | 119.5(2) |
| C(64) | N(4) | C(65) | 115.10(18) | C(54) | C(55) | C(56) | 119.8(2) |
| C(47) | N(3) | C(48) | 117.54(18) | N(2) | C(31) | C(32) | 112.71(18) |
| C(30) | N(2) | C(31) | 116.84(17) | N(2) | C(31) | C(33) | 106.54(17) |
| C(44) | C(43) | C(42) | 119.52(19) | N(2) | C(31) | C(34) | 108.73(18) |
| C(38) | C(43) | C(44) | 118.3(2) | C(33) | C(31) | C(32) | 109.91(19) |
| C(38) | C(43) | C(42) | 122.16(19) | C(34) | C(31) | C(32) | 109.56(19) |
| C(27) | C(26) | C(25) | 119.54(19) | C(34) | C(31) | C(33) | 109.30(19) |
| C(21) | C(26) | C(27) | 118.5(2) | C(9) | C(4) | C(3) | 120.3(2) |
| C(21) | C(26) | C(25) | 122.0(2) | C(9) | C(4) | C(5) | 121.0(2) |
| O(11) | C(44) | C(43) | 114.81(19) | C(3) | C(4) | C(5) | 118.7(2) |
| O(11) | C(44) | C(35) | 123.7(2) | C(43) | C(38) | C(37) | 119.7(2) |
| C(35) | C(44) | C(43) | 121.5(2) | C(43) | C(38) | C(39) | 120.8(2) |
| O(4) | C(12) | C(11) | 108.80(19) | C(37) | C(38) | C(39) | 119.5(2) |
| O(4) | C(12) | C(13) | 111.82(19) | O(10) | C(41) | C(42) | 108.11(16) |
| C(11) | C(12) | C(13) | 110.1(2) | O(10) | C(41) | C(40) | 110.82(19) |
| O(8) | C(29) | C(28) | 110.38(17) | C(40) | C(41) | C(42) | 109.70(19) |
| O(8) | C(29) | C(30) | 106.67(17) | O(15) | C(62) | C(63) | 105.75(18) |
| C(30) | C(29) | C(28) | 112.65(18) | N(3) | C(48) | C(49) | 112.25(19) |
| O(7) | C(27) | C(26) | 114.50(19) | N(3) | C(48) | C(51) | 108.68(19) |
| O(7) | C(27) | C(18) | 124.1(2) | N(3) | C(48) | C(50) | 106.69(19) |
| C(18) | C(27) | C(26) | 121.4(2) | C(51) | C(48) | C(49) | 110.3(2) |
| O(11) | C(45) | C(46) | 106.80(19) | C(50) | C(48) | C(49) | 109.88(19) |
| O(7) | C(28) | C(29) | 105.99(17) | C(50) | C(48) | C(51) | 108.92(19) |
| N(1) | C(14) | C(15) | 106.64(19) | C(60) | C(59) | C(58) | 111.35(19) |
| N(1) | C(14) | C(17) | 112.94(19) | N(2) | C(30) | C(29) | 109.67(17) |
| N(1) | C(14) | C(16) | 108.34(19) | N(4) | C(64) | C(63) | 111.61(18) |
| C(15) | C(14) | C(17) | 109.7(2) | O(3) | C(11) | C(12) | 107.49(18) |
| C(15) | C(14) | C(16) | 109.2(2) | O(9) | C(40) | C(41) | 109.2(2) |
| C(16) | C(14) | C(17) | 109.9(2) | O(9) | C(40) | C(39) | 110.0(2) |
| N(4) | C(65) | C(66) | 106.89(19) | C(39) | C(40) | C(41) | 110.05(18) |
| N(4) | C(65) | C(68) | 112.89(19) | N(1) | C(13) | C(12) | 111.11(19) |
| N(4) | C(65) | C(67) | 107.95(19) | N(3) | C(47) | C(46) | 109.23(18) |
| C(66) | C(65) | C(68) | 110.3(2) | C(2) | C(3) | C(4) | 120.2(2) |
| C(66) | C(65) | C(67) | 109.0(2) | C(37) | C(36) | C(35) | 120.4(2) |
| C(67) | C(65) | C(68) | 109.7(2) | C(61) | C(52) | C(53) | 118.7(2) |
| C(9) | C(8) | C(7) | 112.74(18) | O(12) | C(46) | C(45) | 109.4(2) |
| O(16) | C(63) | C(62) | 107.41(19) | O(12) | C(46) | C(47) | 107.82(19) |
| O(16) | C(63) | C(64) | 111.86(18) | C(47) | C(46) | C(45) | 112.87(19) |
| C(62) | C(63) | C(64) | 110.04(19) | O(1) | C(6) | C(7) | 112.27(19) |
| O(6) | C(24) | C(23) | 110.42(19) | O(1) | C(6) | C(5) | 107.87(19) |
| O(6) | C(24) | C(25) | 108.43(16) | C(5) | C(6) | C(7) | 108.25(19) |
| C(23) | C(24) | C(25) | 110.06(19) | C(2) | C(1) | C(10) | 119.3(2) |
| C(44) | C(35) | C(36) | 119.0(2) | O(14) | C(58) | C(59) | 107.74(18) |
| C(27) | C(18) | C(19) | 118.9(2) | O(14) | C(58) | C(57) | 111.4(2) |
| C(10) | C(9) | C(8) | 119.3(2) | C(59) | C(58) | C(57) | 107.9(2) |
| C(4) | C(9) | C(8) | 122.0(2) | O(2) | C(7) | C(8) | 108.82(18) |
| C(4) | C(9) | C(10) | 118.6(2) | O(2) | C(7) | C(6) | 112.19(18) |
| O(15 | C(61) | C(60) | 113.5(2) | C(8) | C(7) | C(6) | 109.63(18) |
| O(15 | C(61) | C(52) | 125.2(2) | C(57) | C(56) | C(55) | 112.8(2) |
| C(52) | C(61) | C(60) | 121.3(2) | C(19) | C(20) | C(21) | 120.8(2) |
| O(3) | C(10) | C(9) | 114.72(19) | C(36) | C(37) | C(38) | 120.9(2) |
| O(3) | C(10) | C(1) | 124.2(2) | C(4) | C(5) | C(6) | 112.15(19) |
| C(1) | C(10) | C(9) | 121.0(2) | C(21) | C(22) | C(23) | 113.50(19) |
| O(5) | C(23) | C(24) | 108.93(19) | O(13) | C(57) | C(58) | 111.8(2) |
| O(5) | C(23) | C(22) | 110.6(2) | O(13) | C(57) | C(56) | 109.7(2) |
| C(24) | C(23) | C(22) | 110.43(18) | C(58) | C(57) | C(56) | 109.34(19) |
| C(43) | C(42) | C(41) | 112.71(19) | C(54) | C(53) | C(52) | 120.7(2) |
| C(26) | C(25) | C(24) | 112.90(19) | C(20) | C(19) | C(18) | 120.5(2) |
| C(26) | C(21) | C(22) | 121.0(2) | C(53) | C(54) | C(55) | 120.6(2) |
| C(20) | C(21) | C(26) | 119.7(2) | C(40) | C(39) | C(38) | 113.7(2) |
| C(20) | C(21) | C(22) | 119.3(2) | C(3) | C(2) | C(1) | 120.5(2) |

TABLE 6
Hydrogen Bonds.

| D | H | A | d(D-H)/Å | d(H-A)/Å | d(D-A)/Å | D-H-A/° |
|---|---|---|---|---|---|---|
| O(4) | H(4) | O(5) | 0.82 | 1.94 | 2.752(2) | 169.8 |
| O(8) | H(8) | O(11)¹ | 0.82 | 2.24 | 3.038(2) | 163.9 |
| O(6) | H(6A | N(1) | 0.82 | 1.96 | 2.776(2) | 174.1 |
| O(1) | H(1) | N(2)² | 0.82 | 2.00 | 2.777(2) | 158.5 |
| O(10 | H(10 | N(4)³ | 0.82 | 1.92 | 2.738(2) | 173.2 |
| O(2) | H(2) | O(14)³ | 0.82 | 2.15 | 2.967(2) | 171.7 |
| O(5) | H(5) | O(10) | 0.82 | 1.84 | 2.636(2) | 164.3 |
| O(16 | H(16 | O(9)⁴ | 0.82 | 1.91 | 2.717(3) | 168.7 |
| O(14 | H(14 | N(3) | 0.82 | 1.92 | 2.739(2) | 178.6 |
| O(13 | H(13 | O(1)⁵ | 0.82 | 2.06 | 2.876(2) | 173.6 |
| O(9) | H(9) | O(6)⁶ | 0.82 | 1.86 | 2.659(2) | 166.1 |
| O(12) | H(12) | O(13) | 0.82 | 2.56 | 3.116(4) | 125.9 |

¹1 + X, +Y, +Z;
²1 − X, 1/2 + Y, 1 − Z;
³−X, 1/2 + Y, 1 − Z;
⁴−X, −1/2 + Y, 1 − Z;
⁵1 − X, −1/2 + Y, 1 − Z;
⁶−1 + X, +Y, +Z

TABLE 7
Torsion angles [deg]

| A | B | C | D | Angle/° | A | B | C | D | Angle/° |
|---|---|---|---|---|---|---|---|---|---|
| O(11) | C(44) | C(35) | C(36) | −177.4(2) | C(42) | C(43) | C(44) | O(11) | −5.2(3) |
| O(11) | C(45) | C(46) | O(12) | 155.8(2) | C(42) | C(43) | C(44) | C(35) | 175.6(2) |
| O(11) | C(45) | C(46) | C(47) | −84.2(2) | C(42) | C(43) | C(38) | C(37) | −176.6(2) |
| O(3) | C(10) | C(1) | C(2) | 176.8(3) | C(42) | C(43) | C(38) | C(39) | 2.4(3) |
| O(7) | C(27) | C(18) | C(19) | −178.2(2) | C(42) | C(41) | C(40) | O(9) | −174.72(18) |
| O(4) | C(12) | C(11) | O(3) | −56.4(2) | C(42) | C(41) | C(40) | C(39) | 64.5(2) |
| O(4) | C(12) | C(13) | N(1) | −55.0(2) | C(25) | C(26) | C(27) | O(7) | −2.5(3) |
| O(8) | C(29) | C(28) | O(7) | 160.31(17) | C(25) | C(26) | C(27) | C(18) | 177.0(2) |
| O(8) | C(29) | C(30) | N(2) | −70.3(2) | C(25) | C(26) | C(21) | C(20) | −178.8(2) |
| O(6) | C(24) | C(23) | O(5) | 65.5(2) | C(25) | C(26) | C(21) | C(22) | 0.2(3) |
| O(6) | C(24) | C(23) | C(22) | −56.1(2) | C(25) | C(24) | C(23) | O(5) | −174.83(18) |
| O(6) | C(24) | C(25) | C(26) | 73.0(2) | C(25) | C(24) | C(23) | C(22) | 63.6(2) |
| O(1) | C(6) | C(7) | O(2) | −68.9(2) | C(21) | C(26) | C(27) | O(7) | 176.82(19) |
| O(1) | C(6) | C(7) | C(8) | 52.1(2) | C(21) | C(26) | C(27) | C(18) | −3.7(3) |
| O(1) | C(6) | C(5) | C(4) | −68.8(2) | C(21) | C(26) | C(25) | C(24) | 16.7(3) |
| O(10) | C(41) | C(40) | O(9) | 66.0(2) | C(21) | C(20) | C(19) | C(18) | −2.5(4) |
| O(10) | C(41) | C(40) | C(39) | −54.8(2) | C(60) | C(61) | C(52) | C(53) | 0.2(4) |
| O(15) | C(61) | C(60) | C(55) | 178.8(2) | C(60) | C(55) | C(56) | C(57) | 11.9(3) |
| O(15) | C(61) | C(60) | C(59) | −2.9(3) | C(60) | C(55) | C(54) | C(53) | −0.7(4) |
| O(15) | C(61) | C(52) | C(53) | 179.9(3) | C(60) | C(59) | C(58) | O(14) | 67.1(2) |
| O(5) | C(23) | C(22) | C(21) | −166.87(18) | C(60) | C(59) | C(58) | C(57) | −53.3(3) |
| O(16) | C(63) | C(62) | O(15) | −56.1(2) | C(55) | C(60) | C(59) | C(58) | 19.1(3) |
| O(16) | C(63) | C(64) | N(4) | −51.7(2) | C(55) | C(56) | C(57) | O(13) | −169.68(19) |
| O(14) | C(58) | C(57) | O(13) | 72.1(2) | C(55) | C(56) | C(57) | C(58) | −46.7(3) |
| O(14) | C(58) | C(57) | C(56) | −49.6(2) | C(31) | N(2) | C(30) | C(29) | 171.86(17) |
| O(9) | C(40) | C(39) | C(38) | −166.87(19) | C(4) | C(9) | C(10) | O(3) | −176.6(2) |
| N(3) | C(47) | C(46) | O(12) | −62.9(3) | C(4) | C(9) | C(10) | C(1) | 2.5(3) |
| N(3) | C(47) | C(46) | C(45) | 176.13(18) | C(4) | C(3) | C(2) | C(1) | 0.6(4) |
| C(43) | C(44) | C(35) | C(36) | 1.7(3) | C(38) | C(43) | C(44) | O(11) | 175.38(19) |
| C(43) | C(42) | C(41) | O(10) | 73.0(2) | C(38) | C(43) | C(44) | C(35) | −3.8(3) |
| C(43) | C(42) | C(41) | C(40) | −47.9(2) | C(38) | C(43) | C(42) | C(41) | 15.3(3) |
| C(43) | C(38) | C(37) | C(36) | 0.2(4) | C(41) | C(40) | C(39) | C(38) | −46.5(3) |
| C(43) | C(38) | C(39) | C(40) | 13.6(3) | C(62) | O(15) | C(61) | C(60) | 173.4(2) |
| C(26) | C(27) | C(18) | C(19) | 2.4(4) | C(62) | O(15) | C(61) | C(52) | −6.4(4) |
| C(26) | C(21) | C(20) | C(19) | 1.2(4) | C(62) | C(63) | C(64) | N(4) | −171.02(18) |
| C(26) | C(21) | C(22) | C(23) | 14.9(3) | C(48) | N(3) | C(47) | C(46) | 176.30(18) |
| C(44) | O(11) | C(45) | C(46) | — | C(59) | C(60) | C(55) | C(56) | 2.2(4) |
| C(44) | C(43) | C(42) | C(41) | — | C(59) | C(60) | C(55) | C(54) | −176.6(2) |
| C(44) | C(43) | C(38) | C(37) | 2.8(3) | C(59) | C(58) | C(57) | O(13) | −169.81(19) |
| C(44) | C(43) | C(38) | C(39) | −178.2(2) | C(59) | C(58) | C(57) | C(56) | 68.5(2) |
| C(44) | C(35) | C(36) | C(37) | 1.4(4) | C(30) | N(2) | C(31) | C(32) | −62.8(2) |
| C(27) | O(7) | C(28) | C(29) | — | C(30) | N(2) | C(31) | C(33) | 176.61(18) |
| C(27) | C(26) | C(25) | C(24) | — | C(30) | N(2) | C(31) | C(34) | 58.9(3) |
| C(27) | C(26) | C(21) | C(20) | 1.9(3) | C(30) | C(29) | C(28) | O(7) | −80.6(2) |
| C(27) | C(26) | C(21) | C(22) | −179.1(2) | C(64) | N(4) | C(65) | C(66) | −171.90(18) |
| C(27) | C(18) | C(19) | C(20) | 0.7(4) | C(64) | N(4) | C(65) | C(68) | −50.5(3) |
| C(45) | O(11) | C(44) | C(43) | 164.44(19) | C(64) | N(4) | C(65) | C(67) | 70.9(2) |
| C(45) | O(11) | C(44) | C(35) | −16.4(3) | C(64) | C(63) | C(62) | O(15) | 65.9(2) |
| C(28) | O(7) | C(27) | C(26) | 176.16(19) | C(11) | O(3) | C(10) | C(9) | 169.7(2) |
| C(28) | O(7) | C(27) | C(18) | −3.3(3) | C(11) | O(3) | C(10) | C(1) | −9.4(3) |
| C(28) | C(29) | C(30) | N(2) | 168.43(17) | C(11) | C(12) | C(13) | N(1) | −176.11(19) |
| C(14) | N(1) | C(13) | C(12) | 179.67(18) | C(13) | N(1) | C(14) | C(15) | −169.60(19) |
| C(65) | N(4) | C(64) | C(63) | 179.25(18) | C(13) | N(1) | C(14) | C(17) | −49.0(3) |
| C(8) | C(9) | C(10) | O(3) | 1.7(3) | C(13) | N(1) | C(14) | C(16) | 72.9(3) |
| C(8) | C(9) | C(10) | C(1) | −179.2(2) | C(13) | C(12) | C(11) | O(3) | 66.5(2) |
| C(8) | C(9) | C(4) | C(3) | −179.5(2) | C(47) | N(3) | C(48) | C(49) | −66.2(2) |
| C(8) | C(9) | C(4) | C(5) | 0.3(3) | C(47) | N(3) | C(48) | C(51) | 56.1(3) |
| C(24) | C(23) | C(22) | C(21) | −46.2(3) | C(47) | N(3) | C(48) | C(50) | 173.44(18) |
| C(35) | C(36) | C(37) | C(38) | −2.4(4) | C(3) | C(4) | C(5) | C(6) | 159.1(2) |
| C(9) | C(8) | C(7) | O(2) | 169.21(18) | C(52) | C(61) | C(60) | C(55) | −1.4(4) |
| C(9) | C(8) | C(7) | C(6) | 46.2(2) | C(52) | C(61) | C(60) | C(59) | 176.9(2) |
| C(9) | C(10) | C(1) | C(2) | −2.2(4) | C(52) | C(53) | C(54) | C(55) | −0.6(4) |
| C(9) | C(4) | C(3) | C(2) | −0.2(4) | C(7) | C(8) | C(9) | C(10) | 168.3(2) |
| C(9) | C(4) | C(5) | C(6) | −20.7(3) | C(7) | C(8) | C(9) | C(4) | −13.4(3) |
| C(61) | O(15) | C(62) | C(63) | −166.4(2) | C(7) | C(6) | C(5) | C(4) | 52.9(3) |
| C(61) | C(60) | C(55) | C(56) | −179.6(2) | C(56) | C(55) | C(54) | C(53) | −179.5(2) |
| C(61) | C(60) | C(55) | C(54) | 1.6(4) | C(20) | C(21) | C(22) | C(23) | −166.1(2) |
| C(61) | C(60) | C(59) | C(58) | −159.1(2) | C(37) | C(38) | C(39) | C(40) | −167.3(2) |
| C(61) | C(52) | C(53) | C(54) | 0.8(4) | C(5) | C(4) | C(3) | C(2) | 180.0(2) |
| C(10) | O(3) | C(11) | C(12) | −155.4(2) | C(5) | C(6) | C(7) | O(2) | 172.11(18) |
| C(10) | C(9) | C(4) | C(3) | −1.3(3) | C(5) | C(6) | C(7) | C(8) | −66.9(2) |
| C(10) | C(9) | C(4) | C(5) | 178.5(2) | C(22) | C(21) | C(20) | C(19) | −177.8(2) |
| C(10) | C(1) | C(2) | C(3) | 0.6(4) | C(54) | C(55) | C(56) | C(57) | −169.3(2) |
| C(23) | C(24) | C(25) | C(26) | −47.9(2) | C(39) | C(38) | C(37) | C(36) | −178.8(2) |

### Example 3: In Vivo PK in Rats

The rat pharmacokinetics studies of exemplary compounds NI-C and NI-D were conducted with male SD rats. These rats were typically about 6-8 weeks old, weighing 200 g to 300 g. Animals were fasted overnight and have free access to food 4 hours after dosing. A required volume of vehicle was added to reach the target concentration of the test article and vehicle component to prepare for the dosing. The dosing vehicle was PEG400 or 30% PEG400 in saline for IV, or 0.5% methylcellulose in water for PO. For IV dosing, the animals were administered intravenously via tail vein. For PO dosing, the animals were administered via oral gavage. After dosing, blood sample was collected (~0.2 mL per time point) through jugular vein or heart puncture. Blood of each sample was transferred into plastic micro centrifuge tubes containing EDTA-K2. Collection tubes with blood samples and anticoagulant were inverted several times for proper mixing of the tube contents and then placed on wet ice. The blood samples were centrifuged at 2000 g for 5 minutes at 4° C. to obtain plasma which was stored in a freezer at −75±15° C. prior to analysis. Samples were analyzed using LC-MS/MS. WinNonlin were used for pharmacokinetic calculations.

| | rat PK (1 mg/kg), iv | | | | | rat PK (5 mg/kg), po | | | | |
|---|---|---|---|---|---|---|---|---|---|---|
| | CL | T_{1/2} | C₀ | AUC_{Inf} | Vₛₛ | Cₘₐₓ | Tₘₐₓ | MRT | AUC_{Inf} | |
| | (mL/min/kg) | (h) | (ng/mL) | (h*ng/mL) | (L/kg) | (ng/mL) | (h) | (h) | (h*ng/mL) | Bioavailability |
| NI-C | 34.5 | 2.87 | 1142 | 488 | 4.74 | 37.9 | 1.08 | 4.12 | 200 | 8.21% |

### Example 4: In Vitro Pharmacology of Nadolol and Isomers

Affinity estimates calculated using the Leff-Dougal equation (Leff, P. & Dougall, I. G. Further concerns over Cheng-Prusoff analysis. Trends Pharmacol Sci 14, 110-112 (1993)) from pIC50s and Hill slopes generated in CHO-K1 cells expressing beta adrenergic receptors, after cAMP stimulation by approximate EC₈₀ concentrations of isoproterenol.

TABLE 8
pKi values of Nadolol and NI-A through NI-D, with Ki values in parentheses.

| Receptor | Nadolol | NI-A | NI-B | NI-C | NI-D |
|---|---|---|---|---|---|
| β1-AR | 8.26 ± 0.11 | 6.05 ± 0.10 | 6.45 ± 0.10 | 8.34 ± 0.12 | 8.58 ± 0.18 |
| | (5.5 nM) | (891 nM) | (354 nM) | (4.6 nM) | (2.6 nM) |
| β2-AR | 9.26 ± 0.10 | 7.37 ± 0.10 | 7.76 ± 0.13 | 9.39 ± 0.10 | 9.72 ± 0.11 |
| | (0.55 nM) | (43 nM) | (17 nM) | (0.41 nM) | (0.19 nM) |

### Methods

cAMP Assay Protocol

β-AR activation causes Gs activation and subsequent Gs-mediated adenylate cyclase (AC) activation, which in turn increases cAMP production (Alexander et al., 2017). When cAMP turnover is reduced by blocking phosphodiesterases with IBMX, cAMP accumulation occurs, and this accumulation is detected in the cAMP Gs Homogenous Time Resolved Fluorescence (HTRF) assay. The HTRF assay is a competitive inhibition assay. Endogenous cAMP, produced by stimulated cells, competes with and prevents FRET between acceptor-labeled cAMP and donor-labeled cAMP antibody. Increases in endogenous cAMP therefore cause a reduction in HTRF signal, expressed as a ratio of FRET acceptor fluorescence and FRET donor fluorescence.

cAMP HTRF measurements were performed broadly according to the cAMP Gs dynamic kit protocol (Cisbio/Perkin Elmer, 62AM4PEC). Compounds were prepared at 2× final concentration to accommodate the subsequent addition of cells. To prepare compound dose-response curves and inhibition curves, compounds were diluted to the necessary highest concentration in 1× stimulation buffer containing 500 μM 3-isobutyl-1-methylxanthine (IBMX, a phosphodiesterase inhibitor to prevent cAMP inactivation) and dispensed into a 96 well U-bottom polypropylene assay plate (Corning 3365). Vehicle (DMSO), a maximally active isoproterenol dose (0.1 μM for β₁-AR, β₂-AR, 1 μM for β₃-AR) and a full isoproterenol dose response curve were added as controls on all assay plates. Test compounds were serially diluted across the plate by nine 5-fold dilutions to generate a 10-point dose-response curve.

5 μL from 96-well compound source plates was stamped into every well of a 384-well white-bottom plate (Corning 3825) using a viaFLO 384 equipped with a 0.5-12.5 μL pipetting head (Integra), thus creating 4 technical replicates for each treatment condition. 384 well plates containing compound were covered and stored at room temperature until resuspended cell addition.

### Preparation of HTRF Detection Reagents

D2-labelled cAMP (Acceptor) and Europium cryptate labelled cAMP antibody (Donor) (Cisbio/Perkin Elmer 62AM4PEC) were dissolved in water following the manufacturer's instructions, aliquoted and stored at −80° C. For detection of cAMP, on the day of an assay, both reagents were diluted 21-fold in provided lysis buffer (Cisbio/Perkin Elmer) and combined before addition to assay plates.

Stimulation and Quantification of Cellular cAMP (Gs cAMP Assay)

CHO-K1 cells expressing exogenous β-ARs were resuspended using methods as described above, were recovered from flasks using PBS containing magnesium and calcium (Caisson PBL02) and centrifuged at 250×g for 5 minutes in a 50 ml falcon tube. PBS/versene was removed by aspiration and cells were resuspended in a minimal volume of room temperature 1× stimulation buffer+IBMX prior to cell counting. 5 ul cells of correct density (2000 cells/well) were added to compound in assay plates using a Multidrop Combi (Thermofisher). Cells and compounds were covered with a clear plate seal (Axygen PCR-SP) and incubated at 37° C., 5% CO₂ for 30 minutes. cAMP accumulation was then stopped by adding 10 μL HTRF detection reagents in lysis buffer using the Multidrop Combi, plates were covered with an aluminum plate seal (Axygen PCR-AS-600) and agitated (Heidolph Titramax 1000, setting 600) for at least 2 hours, to allow competition between D2-cAMP and stimulated cAMP for the donor-labelled cAMP antibody to reach equilibrium.

### Measurement of HTRF

HTRF was detected using a Tecan Spark plate reader, operated using SparkControl™ software, using the TR fluorescence intensity setting. Both donor and acceptor was excited at 320 nm, detected after 100 μs delay, with 400 μs integration time, and 50 flashes per read. Donor fluorescence was detected at 620 nm and acceptor fluorescence was detected at 665 nm. To standardize reads across experiments, camera gain was manually set to 120 for donor detection, and 140 for acceptor detection.

Table 8 shows NADOLOL and (SRS)-NADOLOL appear to be substrates for OATP1A2. Uptake of NADOLOL was 3.35 fold, 2.08-fold, and 1.78-fold higher in OATP1A2-transfected cells than in the corresponding control cells for 3 μM, 10 μM, and 30 μM, respectively, while uptake of (SRS)-NADOLOL was 5.64-fold, 2.55-fold, and 4.69-fold higher in OATP1A2-transfected cells than in the corresponding control cells. In the presence of a reference inhibitor, nearly complete inhibition of this uptake was seen, confirming that NADOLOL and (SRS)-NADOLOL are substrates for OATP1A2. In contrast, (SSR)-NADOLOL does not appear to be a substrate for OATP1A2, as uptake for 3 μM, 10 μM, and 30 μM (SSR)-NADOLOL was only 1.60-fold, 1.80-fold, and 1.42-fold higher, respectively, in OATP1A2-transfected cells compared to the corresponding control cells, less than the 2-fold threshold used to determine whether it would be a substrate.

### Example 5: Reduced CNS Penetration of Ni-C Vs Nadolol

Escalating doses of Compound 03-5 and nadolol were administered to healthy human subjects under fasted conditions once daily for 7 days according to the Table 9. Nadolol was given 2 hours prior to the dose of Compound 03-5. CSF samples were collected from 3 subjects on Day 2 and from 4 subjects on Day 6 at 2 hours after dosing Compound 03-5 and blood samples were collected on Days 1 through 7 within 1 hour prior to first dose of nadolol, within 0.5 hour prior to first dose of Compound 03-5 and after the first dose of Compound 03-5 at hour 0.25±0.1, 0.5±0.1, 1±0.25, 2±0.5, 4±0.5, 6±1, and 12±1.

TABLE 9

| | Day 1 | Day 2 | Day 3 | Day 4 | Day 5 | Day 6 | Day 7 |
|---|---|---|---|---|---|---|---|
| Compound 03-5 | 3 mg | 3 mg | 3 mg | 6 mg | 15 mg | 15 mg | 15 mg |
| Nadolol | 1 mg | 2 mg | 10 mg | 10 mg | 10 mg | 20 mg | 40 mg |

CSF and blood samples were processed according to common lab protocols and analyzed by following procedures similar to described in literature (see Lee et al, Analytical Sci. and Procedure, 2020, Volume 33, Pages 59-67). Concentrations of nadolol isomers in CSF and plasma collected on Day 6 at 2 hrs after dosing Compound 03-5 are summarized in Table 9. Data suggests that nadolol isomers have similar exposure in plasma and NI-C likely has lower penetration potential to CSF and central nervous system.

TABLE 10

| | Cpd 03-5 | Nadolol | NI-C | NI-D | NI-A | NI-B |
|---|---|---|---|---|---|---|
| | Concentration in CSF (ng/mL) | | | | | |
| Subject A | 37 | 0.607 | 0.114 | 0.178 | 0.116 | 0.177 |
| Subject B | 27.1 | 0.772 | 0.0654 | 0.251 | 0.0984 | 0.33 |
| Subject C | 30.3 | 0.374 | 0.0371 | 0.111 | 0.0554 | 0.134 |
| Subject D | 44.3 | 0.459 | 0.0472 | 0.144 | 0.0706 | 0.164 |
| Average | 34.675 | 0.553 | 0.0659 | 0.171 | 0.0851 | 0.2013 |
| | Concentration in plasma (ng/mL) | | | | | |
| Subject A | 87.4 | 22.9 | 6.02 | 6.21 | 5.78 | 4.03 |
| Subject B | 61.2 | 18.3 | 4.8 | 5.57 | 5.34 | 4.03 |
| Subject C | 72.9 | 11.8 | 2.77 | 3.17 | 2.84 | 2.27 |
| Subject D | 75.5 | 25.1 | 5.31 | 5.83 | 6.09 | 4.46 |
| Average | 74.25 | 19.525 | 4.725 | 5.195 | 5.0125 | 3.6975 |
| | CSF/Plasma ratio | | | | | |
| Subject A | 42% | 2.7% | 1.90% | 2.90% | 2.00% | 4.40% |
| Subject B | 44% | 4.2% | 1.40% | 4.50% | 1.80% | 8.20% |
| Subject C | 42% | 3.2% | 1.30% | 3.50% | 2.00% | 5.90% |
| Subject D | 59% | 1.8% | 0.90% | 2.50% | 1.20% | 3.70% |
| Average | 47% | 3% | 1.40% | 3.30% | 1.70% | 5.50% |

Measured concentrations of Nadolol and isomers in CSF and in the periphery (plasma), as shown in Table 10.

Lower CSF: Plasma ratio of NI-C vs Nadolol, as shown in table 10, NI-C has lower CSF penetrance (reduced CSF: Plasma ratio) vs Nadolol (*p<0.05, Paired t test)

Calculated β2-AR receptor occupancy (fraction of β2-AR receptors bound) by Nadolol and isomers in the CSF and in the periphery. After 20 mg PO dose of Nadolol administered to four human subjects. NI-C has reduced β2-AR receptor occupancy in the CSF compared to Nadolol (33% vs 75%) while maintaining at least 97% peripheral (plasma) occupancy (Table 10).

Method: β2-AR occupancy for Nadolol and each isomer was calculated using the formula [A]/([A]+Ki), where [A] is the concentration of the compound and Ki is compound affinity (see: Gaddum J H. The quantitative effects of antagonistic drugs. J. Physiol. 1937; 89:7-9P) (Table 11).

TABLE 11
Percentage and standard deviation Nadolol or Nadolol isomer
β2-AR occupancy in the CSF or periphery. Percentage occupancy
is the fraction of β2-AR receptors bound multiplied by 100.

| Receptor | Nadolol | NI-A | NI-B | NI-C | NI-D |
|---|---|---|---|---|---|
| Percentage | 75% ± 5% | 1% ± 0% | 4% ± 1% | 33% ± 0.11 | 73% ± 7% |
| β2-AR | | | | | |
| occupancy | | | | | |
| in CSF | | | | | |
| Percentage | 99% ± 0% | 26% ± 6% | 40% ± 7% | 97% ± 1% | 99% ± 0% |
| β2-AR | | | | | |
| occupancy | | | | | |
| in Plasma | | | | | |

Model of Nadolol vs NI-C peripheral and CSF β2-AR occupancy (fraction of β2-AR receptors bound). Cmax and Cmin Nadolol and NI-C concentrations from a 1 mg, 3 mg or 10 mg daily dosing schedule are shown as measured in the study detailed in table xx. NI-C displays higher peripheral β2-AR occupancy than Nadolol across all concentrations, as NI-C has higher affinity than Nadolol. Concomitantly, NI-C has lower CSF β2-AR occupancy than Nadolol across all concentrations, due to lower CSF penetrance of NI-C versus Nadolol. Occupancy calculated as [A]/([A]+Ki) as detailed above across a range of modeled Nadolol/Nadolol isomer concentrations.

Modeled CSF vs peripheral β2-AR occupancy difference (CSF minus peripheral occupancy). NI-C occupancy difference is greater than Nadolol over all concentrations. Projected Cmax and Cmin Nadolol and NI-C concentrations from a 1 mg, 3 mg or 10 mg daily dosing schedule are shown.

Modeled β2-AR occupancy of 90 nM of example β2-AR agonist co-dosed with NI-C or Nadolol across a range of NI-C or Nadolol concentrations. Agonist occupancy considering competition with Nadolol of NI-C calculated using the Gaddum equation:

$$\frac{[ AR ]}{[ {R}_{tot} ]} = \frac{[ A ] / {K}_{A}}{[ A ] / {K}_{A} + [ B ] / {K}_{B} + 1}$$

Where [AR]/[R^{tot}] is the fraction of Receptors bound, [A] is the concentration of agonist, K_{A} is the affinity of the agonist, [B] is the concentration of antagonist (Nadolol or isomer) and K_{B} is the affinity of antagonist.

Modeled β2-AR occupancy difference (CSF occupancy minus peripheral occupancy) of 90 nM example β2-AR agonist co-dosed with NI-C or Nadolol across a range of NI-C or Nadolol concentrations. NI-C provides greater occupancy separation than Nadolol and thus provides a greater concentration window for maintaining a difference between agonist β2-AR binding in the CSF vs the periphery. As an example, a difference in agonist CSF minus peripheral β2-AR occupancy of at least 0.3 is achieved by a dose range of 1.5 to 30 nM with NI-C, but only 2.5 to 15 nM with Nadolol.

From the study described above, compound 03-5 β2-AR occupancy was calculated using measured plasma and CSF concentrations of Compound 03-5, Nadolol and Nadolol isomers, using the Gaddum equation detailed above. Compound 03-5 B2-AR affinity was measured by radioligand binding as 374 nM. When considered alone (no nadolol or isomer competition for β2-AR binding), the fraction of β2-AR receptors bound by Compound 03-5 is 0.46 in the periphery and 0.29 in the CSF. CSF occupancy is lower due to 47% CSF: Plasma penetration of Compound 03-5 (Table YY). Nadolol fully blocks the fraction of peripheral 02-AR receptors bound by Compound 03-5 to less than 0.01, but also lowers the fraction of central β2-AR receptors bound by Compound 03-5 to 0.09. In contrast, NI-C blocks the fraction of peripheral β2-AR receptors bound by Compound 03-5 to 0.02 while maintaining the fraction of central β2-AR receptors bound by Compound 03-5 to 0.21.

Assessing the difference between CSF and peripheral β2-AR occupancy by Compound 03-5 confirms that NI-C provides a larger and significant positive differential than Nadolol and other Nadolol isomers. Taken together, NI-C is advantageous over Nadolol in terms of maintaining Compound 03-5 β2-AR CSF occupancy while blocking β2-AR peripheral occupancy (**p<0.01, Paired t test).

### Aspects and Embodiments of the Disclosure

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

All references referred to in the present disclosure are hereby incorporated by reference in their entirety. Various embodiments of the present disclosure may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

The embodiments of the disclosure described above are intended to be merely exemplary; numerous variations and modifications will be apparent to those skilled in the art. All such variations and modifications are intended to be within the scope of the present disclosure as defined in any appended claims.

1-3. (canceled)

4. A pharmaceutical composition comprising a purified NI-D nadolol isomer, wherein said composition is substantially free of one or more nadolol isomers selected from the group selected from NI-A, NI-B, and NI-C.

5-17. (canceled)

18. The pharmaceutical composition of claim 4, wherein the composition is administered to a human subject.

19. The pharmaceutical composition of claim 4, wherein said isomer has less than 35%; or less than 30%; or less than 25%; or less than 20%; or less than 15%; or less than 12%; or less than 10%; or less than 9%; or less than 8%; or less than 7%; or less than 6%; or less than 5%; or less than 4%; or less than 3%; or less than 2%; or less than 1%; or less than 0.5% other mentioned nadolol isomers.

20. The pharmaceutical composition of claim 4, wherein said composition further includes a second agent.

21. The pharmaceutical composition of claim 4, wherein said composition further includes a second agent that is present in a single formulation.

22. The pharmaceutical composition of claim 4, wherein said composition further includes a second agent that is a β agonist.

23. The pharmaceutical composition of claim 4, wherein said composition further includes a second agent that is a β₁-AR agonist.

24. The pharmaceutical composition of claim 4, wherein said composition further includes a second agent that is a β₂-AR agonist.

25. The pharmaceutical composition of claim 4, wherein said composition further includes a second agent that is one or more selected from the group consisting of salbutamol, levosalbutamol, terbutaline, pirbuterol, procaterol, metaproterenol, bitolterol mesylate, oritodrine, isoprenaline, salmefamol, fenoterol, terbutaline, albuterol, isoetharine, salmeterol, bambuterol, formoterol, clenbuterol, indacaterol, vilanterol, olodaterol, tulobuterol, mabuterol, and ritodrine.

26. The pharmaceutical composition of claim 4, wherein said composition further includes a second agent that is a β agent.

27. The pharmaceutical composition of claim 4, wherein said composition further includes a second agent that is clenbuterol.

28. The pharmaceutical composition of claim 4, wherein said composition further includes a second agent that is tulobuterol.

29. The pharmaceutical composition of claim 4, wherein said purified nadolol isomer has improved efficacy than a nadolol racemic mixture.

30. The pharmaceutical composition of claim 4, wherein said purified nadolol isomer has improved gastric uptake than a nadolol racemic mixture.

31. The pharmaceutical composition of claim 4, wherein said purified nadolol isomer has improved gastric uptake than a nadolol racemic mixture in the presence of green tea.

32. A method comprising administering a pharmaceutical composition of claim 4 to a subject.

33. (canceled)

34. A method comprising administering to a subject a composition of claim 4 and a second active agent.

35. The method of claim 34, wherein the second active ingredient is a β-AR agonist.

36. The method of claim 34, wherein the second active ingredient is a β₁-AR agonist.

37. The method of claim 34, wherein the second active ingredient is a β₂-AR agonist.

38. (canceled)

39. A method comprising identifying a subject desiring improvement of cognitive function and/or treatment of a neurodegenerative disease or disorder and administering to said subject a pharmaceutical composition of claim 4.

40. The method of claim 39, wherein the neurodegenerative disease is selected from the group consisting of MCI, aMCI, Vascular Dementia, Mixed Dementia, FTD (fronto-temporal dementia; Pick's disease), ALS (amyotrophoic lateral sclerosis), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CID etc.), depressive disorders, DLB (dementia with Lewy bodies), PD (Parkinson's disease), PDD (PD dementia), ADHD (attention deficit hyperactivity disorder), and Down Syndrome.
